(19) United States
(12) Patent Application Publication
(10) Pub. No.: US 2013/0023563 A1
(43) Pub. Date: Jan. 24, 2013

(54) AMIDINOANILINE DERIVATIVE

(71) Applicant: AJINOMOTO CO., INC., Tokyo (JP)

(72) Inventors: Kayo Matsumoto, Kawasaki-shi (JP); Masayuki Sugiki, Kawasaki-shi (JP); Tatsuhiro Yamada, Kawasaki-shi (JP); Misato Noguchi, Kawasaki-shi (JP); Masaru Takayanagi, Kawasaki-shi (JP); Atsushi Takada, Kawasaki-shi (JP); Tadakiyo Nakagawa, Kawasaki-shi (JP); Wataru Miyanaga, Kawasaki-shi (JP); Shin Muronoi, Kawasaki-shi (JP); Shinya Taniguchi, Kawasaki-shi (JP)

(73) Assignee: AJINOMOTO CO., INC., Tokyo (JP)

(21) Appl. No.: 13/627,251

(22) Filed: Sep. 26, 2012

Related U.S. Application Data
(63) Continuation of application No. PCT/JP2011/057476, filed on Mar. 25, 2011.

(30) Foreign Application Priority Data
Mar. 26, 2010 (JP) 2010-073444

(51) Int. Cl. C07D 295/195; A01N 1/02; C12N 9/99; C07D 211/46; A61K 31/445; C07D 403/12; A61K 31/4025; C07D 401/12; A61K 31/454; C07D 211/60; A61K 31/4545; C07D 413/12; A61K 31/405; A61K 31/4155; A61P 7/02; A61K 31/40

(52) U.S. Cl. 514/316; 548/569; 514/408; 435/2; 435/184; 546/221; 514/327; 548/518; 514/422; 546/208; 514/326; 546/189; 546/198; 514/321; 548/465; 514/414; 546/201; 514/323; 548/364.1; 514/406; 546/211

(57) ABSTRACT

Provided are a novel amidine derivative having an activated blood coagulation factor X inhibitory activity, a production method thereof, a production intermediate therefor, and a pharmaceutical composition containing the amidine derivative. An amidinoaniline derivative represented by the following formula (1-1) or a pharmaceutically acceptable salt thereof:

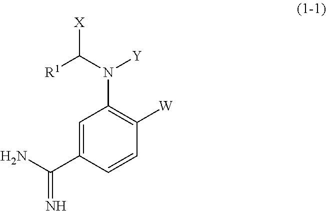

<in the formula (1-1), each symbol is as defined in the Description>, and a pharmaceutical composition containing the amidinoaniline derivative or a pharmaceutically acceptable salt thereof.

### TECHNICAL FIELD

The present invention relates to a novel amidine derivative having an activated blood coagulation factor X (hereinafter sometimes to be abbreviated as FXa) inhibitory activity, a production method thereof, a production intermediate therefor, and a pharmaceutical composition containing the amidine derivative.

The present invention also relates to use of a low molecular weight FXa inhibitor, particularly a low molecular weight FXa inhibitor with a short half-life in blood in the extracorporeal blood circulation circuit and the like.

### BACKGROUND ART

The extracorporeal blood circulation is performed by a circulation circuit that returns blood into the body via an apparatus that performs a given treatment through an artificial channel for blood flow from inside the body to the outside thereof, for example, an artificial heart lung apparatus, a blood purification apparatus and the like. An extracorporeal blood circulation treatment may be necessary in blood purification therapy such as hemodialysis, blood filtration, hemodialysis filtration, plasma exchange and the like, a heart-lung bypass in an open-heart surgery and the like. As a blood purification apparatus, a dialyzer and the like can be typically mentioned.

Upon contact with a foreign substance, the intrinsic blood coagulation cascade is generally activated, and the blood is finally coagulated and loses flowability. An extracorporeal blood circulation circuit comprising an artificial channel for blood flow and various apparatuses for extracorporeal blood circulation is a foreign substance, and the blood coagulates upon contact therewith. Therefore, a treatment by some method to prevent blood coagulation in an extracorporeal blood circulation circuit is necessary.

Conventionally, an anti-(blood) coagulation drug (agent) (anticoagulant) such as unfractionated heparin, low molecular weight heparin and the like has been used for the purpose of preventing blood coagulation in the extracorporeal blood circulation circuit.

However, since unfractionated heparin is known to have a risk of bleeding tendency because of its thrombin inhibitory activity in addition to an FXa inhibitory activity, it cannot be used for patients with a high risk of bleeding. Low molecular weight heparin is a medicament that inhibits FXa more selectively than thrombin due to a chemical treatment applied to heparin, and has a lower risk of bleeding tendency since it is free of a thrombin inhibitory activity. Thus, it is used for patients with bleeding tendency. However, since low molecular weight heparin has a long elimination half-life, hemostasis is difficult when a bleeding symptom is observed.

Moreover, some serine protease inhibitors also have an anticoagulant action and, for example, nafamostat mesylate is used for certain extracorporeal blood circulation such as hemodialysis and the like. Since nafamostat mesylate has a short elimination half-life in the body, it is used for patients already having a bleeding lesion. However, nafamostat mesylate shows a weak anticoagulant effect since it does not have a strong inhibitory activity against FXa and thrombin.

As mentioned above, all medicaments still have problems, and a more effective and safer medicament is desired.

Patients with an extracorporeal circulation circuit are faced with the problem of blood coagulation only during use of the circuit, and are often different from the patients in need of continuous prevention of blood coagulation. A selective low-molecular-weight FXa inhibitor with a short half-life in blood can be safely and conveniently used as an anticoagulant to prevent blood coagulation due to an extracorporeal blood circulation circuit, and a treatment of hemostasis and attention required after completion of the extracorporeal blood circulation can be clearly less. Furthermore, when a compound that rapidly loses activity in the body due to the metabolism by the liver should exit from the extracorporeal circulation circuit and be exposed in the body, the active substance itself is considered to be inactivated in the body due to the metabolism by the liver, and side effects such as bleeding risk and the like are expected to be reduced more, which aspect has not been predicted heretofore.

In addition, as an amidine compound that shows an anticoagulant activity based on an FXa selective inhibitory action, the compounds described in patent documents 1-9 are known. However, they are structurally clearly different from the compound of the present invention.

### DOCUMENT LIST

### Patent Documents

patent document 1: WO98/31661
patent document 2: WO99/64392
patent document 3: WO99/52895
patent document 4: WO99/10316
patent document 5: WO2000/59876
patent document 6: WO2002/28827
patent document 7: WO96/16940
patent document 8: WO2002/42270
patent document 9: WO2006/083003

### SUMMARY OF THE INVENTION

### Problems to be Solved by the Invention

The present invention aims to provide a novel amidine derivative or a pharmaceutically acceptable salt thereof.

The present invention aims to provide a production method of the above-mentioned amidine derivative or a pharmaceutically acceptable salt thereof and a production intermediate therefor.

The present invention also aims to provide an activated blood coagulation factor X inhibitor containing the above-mentioned amidine derivative or a pharmaceutically acceptable salt thereof.

The present invention also aims to provide an anti-(blood) coagulation drug (agent) containing the above-mentioned amidine derivative or a pharmaceutically acceptable salt thereof.

The present invention also aims to provide a pharmaceutical composition containing the above-mentioned amidine derivative or a pharmaceutically acceptable salt thereof.

The present invention also aims to provide a novel anti-(blood) coagulation drug (agent) or a pharmaceutical composition for an extracorporeal blood circulation circuit.

The present invention also aims to provide a novel method of preventing thrombus formation in an extracorporeal blood circulation circuit.

### Means of Solving the Problems

The present inventors have conducted intensive studies in view of the aforementioned situation and found that a particular novel amidine derivative having an ester bond in a molecule, a compound represented by A′-COO—B′ wherein A′ and B′ are organic groups, at least one of them contains an amidino group or guanidino group structure has a superior activated blood coagulation factor X inhibitory activity, a short half-life in blood, and is useful as a blood anticoagulant for an extracorporeal blood circulation circuit, which resulted in the completion of the present invention.

Accordingly, the present invention is as shown below.

[1] an amidinoaniline derivative represented by the following formula (1-1) or a pharmaceutically acceptable salt thereof:

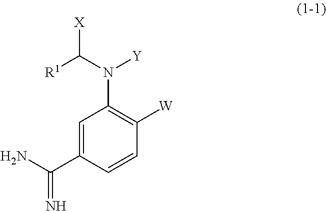

<in the formula (1-1),

X is a hydrogen atom, or a C₁₋₁₀ alkyl group optionally having substituent(s),

Y is a hydrogen atom, a C₁₋₁₀ alkyl group optionally having substituent(s), or an acyl group optionally having substituent(s),

W is a hydrogen atom, a hydroxyl group, an amino group, a C₁₋₁₀ alkyl group optionally having substituent(s), a C₁₋₁₀ alkoxy group optionally having substituent(s), a C₁₋₁₀ acyloxy group optionally having substituent(s), a carbamoyloxy group optionally having substituent(s), a C₁₋₁₀ alkylamino group optionally having substituent(s), a C₁₋₁₀ alkylthio group optionally having substituent(s), a C₁₋₁₀ acylamino group optionally having substituent(s), a carboxyl group, a carbamoyl group optionally having substituent(s), a thiocarbamoyl group optionally having substituent(s), a halogen atom, a cyano group, or a nitro group,

X and Y are optionally bonded to each other to form a nitrogen-containing heterocycle optionally having substituent(s),

Y and W are optionally bonded to each other to form a nitrogen-containing heterocycle optionally having substituent(s),

R¹ is a group represented by the following formula (2-1) or (2-2), provided that when R¹ is a group represented by the formula (2-2), X is not a hydrogen atom,

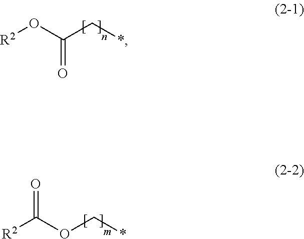

[in the formulas (2-1) and (2-2),

n and m are each an integer of 0-2,

R² is a group represented by the following formula (3):

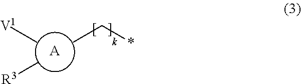

{in the formula (3),

k is an integer of 0-2,

ring A is a C₆₋₁₀ aryl ring, a C₁₋₁₀ heteroaryl ring, a C₂₋₈ nitrogen-containing nonaromatic heterocycle, or a C₃₋₁₀ cycloalkyl ring,

V¹ is a hydrogen atom, a hydroxyl group, a halogen atom, an amino group, a C₁₋₁₀ alkyl group optionally having substituent(s), a C₁₋₁₀ alkoxy group optionally having substituent(s), a C₁₋₁₀ alkylamino group optionally having substituent(s), a C₁₋₁₀ alkylthio group optionally having substituent(s), a cyano group, a nitro group, a carboxyl group, a carbamoyl group optionally having substituent(s) or a C₂₋₁₀ alkoxycarbonyl group optionally having substituent(s),

R³ is a group represented by the following formula (4-1) or (4-2):

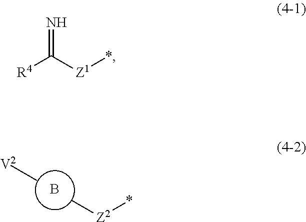

(in the formula (4-1),

Z¹ is —NH— or a single bond,

R⁴ is a C₁₋₆ alkyl group, an amino group optionally substituted by a C₁₋₁₀ alkyl group or a C₂₋₈ nitrogen-containing nonaromatic heterocyclic group bonded by a nitrogen, in the formula (4-2), ring B is a C₁₋₁₀ heteroaryl ring, or a C₂₋₈ nitrogen-containing nonaromatic heterocycle,

Z² is a single bond, —NH— optionally substituted by a C₁₋₆ alkyl group, an oxygen atom, a sulfur atom, a methylene group, or —CO—,

V² is a hydrogen atom, a halogen atom, an amidino group optionally substituted by a C₁₋₆ alkyl group, a guanidino group optionally substituted by a C₁₋₆ alkyl group, or a C₁₋₆ alkyl group optionally having an imino group at the 1-position)}]>.

[2] The amidinoaniline derivative of the above-mentioned [1], wherein, in the formula (1-1), X and Y are each a C₁₋₆ alkyl group optionally having substituent(s), or a pharmaceutically acceptable salt thereof.
[3] An amidinoaniline derivative represented by the following formula (1-2) or a pharmaceutically acceptable salt thereof:

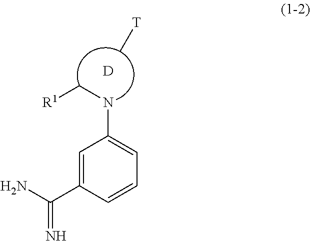

<in the formula (1-2),

R¹ is as defined in the above-mentioned [1],

ring C is a C₂₋₁₀ nitrogen-containing heteroaryl ring, or a C₂₋₈ nitrogen-containing nonaromatic heterocycle,

T is a hydrogen atom, a hydroxyl group, an amino group, a C₁₋₁₀ alkyl group optionally having substituent(s), a C₁₋₁₀ alkoxy group optionally having substituent(s), a C₁₋₁₀ alkylamino group optionally having substituent(s), or a C₁₋₁₀ carbamoyloxy group optionally having substituent(s)>.

[4] An amidinoaniline derivative represented by the following formula (1-3) or a pharmaceutically acceptable salt thereof:

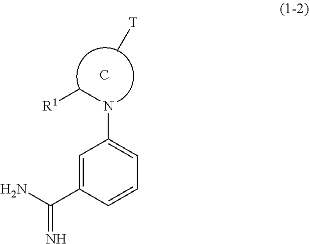

<in the formula (1-3),

R¹ is as defined in the above-mentioned [1],

ring D is a C₂₋₁₀ nitrogen-containing heteroaryl ring, or a C₂₋₈ nitrogen-containing nonaromatic heterocycle>.

[5] The amidinoaniline derivative of the above-mentioned [2], [3] or [4], wherein, in the formula (3),

ring A is a benzene ring, a pyridine ring, a thiophene ring, a piperidine ring, or a piperazine ring, and

V¹ is a hydrogen atom, a halogen atom, a C₁₋₆ alkyl group, or a C₁₋₆ alkoxy group, or a pharmaceutically acceptable salt thereof.

[6] The amidinoaniline derivative of the above-mentioned [5], wherein, in the formula (4-1),

R⁴ is an amino group, a C₁₋₁₀ alkylamino group, or a C₂₋₈ nitrogen-containing nonaromatic heterocyclic group bonded by a nitrogen, or

in the formula (4-2),

ring B is a C₂₋₈ nitrogen-containing nonaromatic heterocycle,

Z² is an oxygen atom, a sulfur atom or a methylene group, and

V² is a hydrogen atom, a halogen atom, an amidino group, or a C₁₋₆ alkyl group optionally having an imino group at the 1-position, or a pharmaceutically acceptable salt thereof.

[7] The amidinoaniline derivative of the above-mentioned [6], wherein, in the formula (3),

ring A is a benzene ring,

R³ is the formula (4-1),

Z¹ is a single bond, and

R⁴ is a C₂₋₈ nitrogen-containing nonaromatic heterocyclic group bonded by a nitrogen, or a pharmaceutically acceptable salt thereof.

[8] An amidinoaniline derivative represented by the following formula (1-1) or a pharmaceutically acceptable salt thereof:

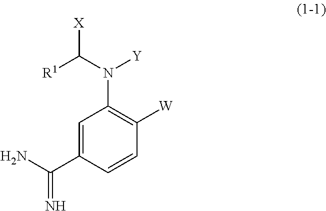

<in the formula (1-1),

X is a hydrogen atom, or a C₁₋₁₀ alkyl group optionally having substituent(s),

Y is a hydrogen atom, a C₁₋₁₀ alkyl group optionally having substituent(s), or an acyl group optionally having substituent(s),

W is a hydrogen atom, a hydroxyl group, an amino group, a C₁₋₁₀ alkyl group optionally having substituent(s), a C₁₋₁₀ alkoxy group optionally having substituent(s), a C₁₋₁₀ acyloxy group optionally having substituent(s), a carbamoyloxy group optionally having substituent(s), a C₁₋₁₀ alkylamino group optionally having substituent(s), a C₁₋₁₀ alkylthio group optionally having substituent(s), a C₁₋₁₀ acylamino group optionally having substituent(s), a carboxyl group, a carbamoyl group optionally having substituent(s), a thiocarbamoyl group optionally having substituent(s), a halogen atom, a cyano group, or a nitro group,

X and Y are optionally bonded to each other to form a nitrogen-containing heterocycle optionally having substituent(s),

Y and W are optionally bonded to each other to form a nitrogen-containing heterocycle optionally having substituent(s),

R¹ is a group represented by the following formula (2-1) or (2-2), provided that when R¹ is a group represented by the formula (2-2), X is not a hydrogen atom,

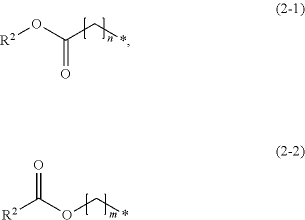

[in the formulas (2-1) and (2-2),

n and m are each an integer of 0-2,

R² is a group represented by the following formula (3′),

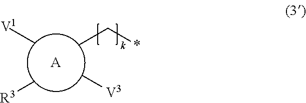

{in the formula (3′),

k is an integer of 0-2,

ring A is a C₆₋₁₀ aryl ring, a C₁₋₁₀ heteroaryl ring, a C₂₋₈ nitrogen-containing nonaromatic heterocycle, or a C₃₋₁₀ cycloalkyl ring,

V¹ and V³ are the same or different and each is a hydrogen atom, a hydroxyl group, a halogen atom, an amino group, a C₁₋₁₀ alkyl group optionally having substituent(s), a C₁₋₁₀ alkoxy group optionally having substituent(s), a C₁₋₁₀ alkylamino group optionally having substituent(s), a C₁₋₁₀ alkylthio group optionally having substituent(s), a cyano group, a nitro group, a carboxyl group, a carbamoyl group optionally having substituent(s) or a C₂₋₁₀ alkoxycarbonyl group optionally having substituent(s),

R³ is a group represented by the following formula (4-1) or (4-2):

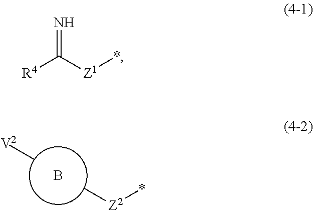

(in the formula (4-1),

Z¹ is —NH—, or a single bond,

R⁴ is a C₁₋₆ alkyl group, an amino group optionally substituted by a C₁₋₁₀ alkyl group, or a C₂₋₈ nitrogen-containing nonaromatic heterocyclic group bonded by a nitrogen, in the formula (4-2),

ring B is a C₁₋₁₀ heteroaryl ring, or a C₂₋₈ nitrogen-containing nonaromatic heterocycle,

Z² is a single bond, —NH— optionally substituted by a C₁₋₆ alkyl group, an oxygen atom, a sulfur atom, a methylene group, or —CO—, and

V² is a hydrogen atom, a halogen atom, an amidino group optionally substituted by a C₁₋₆ alkyl group, a guanidino group optionally substituted by a C₁₋₆ alkyl group, or a C₁₋₆ alkyl group optionally having an imino group at the 1-position)}]>.

[9] An activated blood coagulation factor X inhibitor containing the amidinoaniline derivative of any of the above-mentioned [1]-[8], or a pharmaceutically acceptable salt thereof.
[10] A pharmaceutical composition containing the amidinoaniline derivative of any of the above-mentioned [1]-[8], or a pharmaceutically acceptable salt thereof.
[11] The pharmaceutical composition of the above-mentioned [10], which is an anti-blood coagulant.
[12] The pharmaceutical composition of the above-mentioned [11], which is an anti-blood coagulant for an extracorporeal blood circulation circuit.
[13] The pharmaceutical composition of the above-mentioned [11], which is an anti-blood coagulant for hemodialysis.
[14] A dialysis solution or dialysis concentrate containing the amidinoaniline derivative of any of the above-mentioned [1]-[8], or a pharmaceutically acceptable salt thereof.
[15] An anti-blood coagulant for an extracorporeal blood circulation circuit, which contains a low molecular weight FXa inhibitor as an active ingredient.
[16] The anti-blood coagulant of the above-mentioned [15], wherein the low molecular weight FXa inhibitor rapidly disappears from the blood.
[17] The anti-blood coagulant of the above-mentioned [16], wherein the low molecular weight FXa inhibitor is an FXa selective inhibitor.

The present invention also provides an activated blood coagulation factor X inhibitor, an anti-blood coagulant, or a is pharmaceutical composition, containing the above-mentioned amidinoaniline derivative or a pharmaceutically acceptable salt thereof.

The present invention also provides an anti-(blood) coagulation drug (agent) for an extracorporeal blood circulation circuit, which contains a low molecular weight FXa inhibitor as an active ingredient.

The present invention further provides a method of preventing thrombus formation in an extracorporeal blood circulation circuit, which comprises incorporating a low molecular weight FXa inhibitor as a constituent element of an extracorporeal blood circulation circuit.

### Effect of the Invention

The compound of the present invention has a superior activated blood coagulation factor X inhibitory activity, a short half-life in blood, and is useful as a blood anticoagulant for an extracorporeal blood circulation circuit.

### DESCRIPTION OF EMBODIMENTS

The terms used in the present specification are defined below.

The “aryl ring” means a monocyclic-bicyclic aromatic hydrocarbon ring or benzene ring fused with a 5- to 8-membered cycloalkyl ring. As the “aryl ring”, one having a carbon number of 6-10 is preferable. For example, benzene ring, naphthalene ring, indane ring and tetrahydronaphthalene ring can be mentioned, benzene ring and naphthalene ring are more preferable, and benzene ring is particularly preferable. The “C₆₋₁₀ aryl ring” is one having a carbon number of 6-10 from among the above-mentioned aryl rings.

The “aryl group” is a monocyclic-bicyclic aromatic hydrocarbon ring group, or a group wherein a phenyl group is fused with a 5- to 8-membered cycloalkyl ring. The “aryl group” is preferably one having a carbon number of 6-14. For example, phenyl group, naphthyl group, indanyl group, tetrahydronaphthyl group and the like can be mentioned, more preferably phenyl group and naphthyl group, particularly preferably phenyl group. The “C₆₋₁₄ aryl group” is preferably one having a carbon number of 6-14 from among the above-mentioned “aryl groups”. The “C₆₋₁₀ aryl group” is one having a carbon number of 6-10 from among the above-mentioned “C₆₋₁₄ aryl groups”.

The “heteroaryl ring” is a monocyclic-bicyclic aromatic heterocycle containing, as a ring atom, 1-6 hetero atoms selected from an oxygen atom, a sulfur atom and a nitrogen atom. Examples of the “heteroaryl ring” include pyridine ring, pyridazine ring, pyrimidine ring, pyrazine ring, furan ring, thiophene ring, pyrrole ring, isoxazole ring, oxazole ring, isothiazole ring, thiazole ring, pyrazole ring, imidazole ring, oxadiazole ring, thiadiazole ring, triazole ring, tetrazole ring, benzofuran ring, benzothiophene ring, indoline ring, isoindoline ring, benzoxazole ring (=benzooxazole ring), benzothiazole ring, benzimidazole ring (=benzoimidazole ring), indazole ring, benzisoxazole ring, benzisothiazole ring, benzofurazan ring, benzothiadiazole ring, purine ring, quinoline ring, isoquinoline ring, cinnoline ring, phthalazine ring, quinazoline ring, quinoxaline ring, pteridine ring, imidazooxazole ring, imidazothiazole ring, imidazoimidazole ring and the like. Those having a carbon number of 1-10 are preferable, pyridine ring, pyridazine ring, pyrimidine ring, pyrazine ring, furan ring, thiophene ring, pyrrole ring, isoxazole ring, oxazole ring, isothiazole ring, thiazole ring, pyrazole ring, imidazole ring, oxadiazole ring, thiadiazole ring, triazole ring and tetrazole ring are more preferable, and pyridine ring and thiophene ring are still more preferable. The “C₁₋₁₀ heteroaryl ring” means those having a carbon number of 1-10 from among the above-mentioned “heteroaryl rings”.

The “heteroaryl group” is a monocyclic-bicyclic aromatic heterocyclic group containing, as a ring atom, 1-6 hetero atoms selected from an oxygen atom, a sulfur atom and a nitrogen atom. Examples of the “heteroaryl group” include pyridyl group, pyridazinyl group, pyrimidinyl group, pyrazinyl group, furanyl group, thienyl group, pyrrolyl group, isoxazolyl group, oxazolyl group, isothiazolyl group, thiazolyl group, pyrazolyl group, imidazolyl group, oxadiazolyl group, thiadiazolyl group, triazolyl group, tetrazolyl group, benzofuranyl group, benzothienyl group, indolinyl group, isoindolinyl group, benzoxazolyl group (=benzooxazolyl group), benzothiazolyl group, benzimidazolyl group (=benzoimidazolyl group), indazolyl group, benzisoxazolyl group, benzisothiazolyl group, benzofurazanyl group, benzothiadiazolyl group, purinyl group, quinolinyl group, isoquinolinyl group, cinnolinyl group, phthalazinyl group, quinazolinyl group, quinoxalinyl group, pteridinyl group, imidazooxazolyl group, imidazothiazolyl group, imidazoimidazolyl group and the like. Those having a carbon number of 1-10 are preferable, pyridyl group, pyridazinyl group, pyrimidinyl group, pyrazinyl group, furanyl group, thienyl group, pyrrolyl group, isoxazolyl group, oxazolyl group, isothiazolyl group, thiazolyl group, pyrazolyl group, imidazolyl group, oxadiazolyl group, thiadiazolyl group, triazolyl group, tetrazolyl group are more preferable, and pyridyl group, thienyl group are still more preferable. The “C₁₋₁₀ heteroaryl group” means those having a carbon number of 1-10 from among the above-mentioned “heteroaryl groups”. The “C₁₋₉ heteroaryl group” means those having a carbon number of 1-9 from among the above-mentioned “C₁₋₁₀ heteroaryl groups”.

The “nitrogen-containing nonaromatic heterocycle” is a monocyclic-bicyclic nonaromatic heterocycle containing, as a ring atom, at least one nitrogen atom, and further, not less than one oxygen atom or sulfur atom. Examples of the “nitrogen-containing nonaromatic heterocycle” include pyrrolidine ring, pyrazolidine ring, imidazolidine ring, pyrroline ring, pyrazoline ring, imidazoline ring, oxazolidine ring, 1,3-oxazolidin-2-one ring, thiazolidine ring, piperidine ring, piperidine ring, piperazine ring, quinuclidine ring, morpholine ring, thiomorpholine ring, homopiperidine ring, homopiperazine ring, indoline ring, isoindoline ring, tetrahydroquinoline ring, tetrahydroisoquinoline ring and the like, preferably, pyrrolidine ring, pyrazoline ring, imidazoline ring, 1,3-oxazolidin-2-one ring, thiazolidine ring, piperidine ring, piperazine ring, tetrahydroquinoline ring and tetrahydroisoquinoline ring. Those having a carbon number of 2-8 are preferable, pyrrolidine ring, piperidine ring, piperazine ring, thiazolidine ring and 1,3-oxazolidin-2-one ring are particularly preferable. The “C₂₋₈ nitrogen-containing nonaromatic heterocycle” means those having a carbon number of 2-8 from among the above-mentioned “nitrogen-containing nonaromatic heterocycles”.

The “nitrogen-containing nonaromatic heterocyclic group” is a monocyclic-bicyclic nonaromatic heterocyclic group containing, as a ring atom, at least one nitrogen atom, and further, not less than one oxygen atom or sulfur atom. Examples of the “nitrogen-containing nonaromatic heterocyclic group” include pyrrolidinyl group, pyrazolidinyl group, imidazolidinyl group, pyrrolinyl group, pyrazolinyl group, imidazolyl group, thiazolidinyl group, piperidyl group, piperidino group, piperazinyl group, quinuclidinyl group, morpholino group, morpholinyl group, thiomorpholino group, thiomorpholinyl group, homopiperidyl group, homopiperazinyl group, indolinyl group, isoindolinyl group, tetrahydroquinolinyl group, tetrahydroisoquinolinyl group and the like. Preferred are pyrrolidinyl group, piperidyl group, piperazinyl group, tetrahydroquinolinyl group and tetrahydroisoquinolinyl group. Those having a carbon number of 2-8 are preferable, particularly pyrrolidinyl group, pyrrolinyl group, piperidyl group, piperazinyl group is preferable. The “C₁₋₉ nitrogen-containing nonaromatic heterocyclic group” means those having a carbon number of 1-9 from among the above-mentioned “nitrogen-containing nonaromatic heterocyclic groups”. The “C₂₋₈ nitrogen-containing nonaromatic heterocyclic group” means those having a carbon number of 2-8 from among the above-mentioned “C₁₋₉ nitrogen-containing nonaromatic heterocyclic groups”.

The “nitrogen-containing heterocycle” is a monocyclic-bicyclic heterocycle containing, as a ring atom, at least one nitrogen atom, and further, optionally having 1-6 hetero atoms selected from an oxygen atom and a sulfur atom. The “heterocycle” is a “nitrogen-containing heteroaryl ring” or the above-mentioned “nitrogen-containing nonaromatic heterocycle”, which has a nitrogen atom as a ring atom from among the above-mentioned “heteroaryl rings”. Examples of the “nitrogen-containing heteroaryl ring” include pyridine ring, pyridazine ring, pyrimidine ring, pyrazine ring, pyrrole ring, isoxazole ring, oxazole ring, isothiazole ring, thiazole ring, pyrazole ring, imidazole ring, oxadiazole ring, thiadiazole ring, triazole ring, tetrazole ring, indoline ring, isoindoline ring, benzoxazole ring (=benzooxazole ring), benzothiazole ring, benzimidazole ring (=benzoimidazole ring), indazole ring, benzisoxazole ring, benzisothiazole ring, benzofurazan ring, benzothiadiazole ring, purine ring, quinoline ring, isoquinoline ring, cinnoline ring, phthalazine ring, quinazoline ring, quinoxaline ring, pteridine ring, imidazooxazole ring, imidazothiazole ring, imidazoimidazole ring and the like can be mentioned. Preferred are those having a carbon number of 1-10, and more preferred are pyridine ring, pyridazine ring, pyrimidine ring, pyrazine ring, pyrrole ring, isoxazole ring, oxazole ring, isothiazole ring, thiazole is ring, pyrazole ring, imidazole ring, oxadiazole ring, thiadiazole ring, triazole ring and tetrazole ring. The “C₂₋₁₀ nitrogen-containing heteroaryl ring” means those having a carbon number of 2-10 from among the above-mentioned “nitrogen-containing heteroaryl rings”.

The “nitrogen-containing heterocycle” optionally formed by X and Y bonded to each other is preferably a “C₂₋₁₀ nitrogen-containing heteroaryl ring” or a “C₂₋₈ nitrogen-containing nonaromatic heterocycle”, more preferably a pyrazole ring, a pyrrolidine ring, a piperidine ring, a piperazine ring, a morpholine ring or a thiazolidine ring, still more preferably a pyrrolidine ring or a piperidine ring from among the above-mentioned “nitrogen-containing heterocycles”.

The “nitrogen-containing heterocycle” optionally formed by Y and W bonded to each other is preferably a “C₂₋₁₀ nitrogen-containing heteroaryl ring” or a “C₂₋₈ nitrogen-containing nonaromatic heterocycle”, more preferably a fused ring structure with the adjacent benzene ring such as indole ring, benzimidazole ring (=benzoimidazole ring), indazole ring, indoline ring, 1,2,3,4-tetrahydroquinoline ring and 2,3-dihydro-1,3-benzoxazol-2-one ring, from among the above-mentioned “nitrogen-containing heterocycles”.

The “cycloalkyl ring” means a nonaromatic hydrocarbon ring which may contain a double bond in the ring. As the “cycloalkyl ring”, those having a carbon number of 3-10 are preferable, for example, cyclopropane ring, cyclobutane ring, cyclopentane ring, cyclohexane ring, cycloheptane ring, cyclohexene ring, cyclopentene ring and the like can be mentioned, particularly preferably cyclohexene ring. The “C₃₋₁₀ cycloalkyl ring” means those having a carbon number of 3-10 from among the above-mentioned “cycloalkyl rings”.

The “cycloalkyl group” means a nonaromatic hydrocarbon ring group which may contain a double bond in the ring. As the “cycloalkyl group”, those having a carbon number of 3-10 are preferable, for example, cyclopropyl group, cyclobutyl group, cyclopentyl group, cyclohexyl group, cycloheptyl group, cyclohexenyl group, cyclopentenyl group and the like can be mentioned, particularly preferably cyclohexyl group. The “C₃₋₁₀ cycloalkyl group” means those having a carbon number of 3-10 from among the above-mentioned “cycloalkyl groups”. The “C₃₋₈ cycloalkyl group” means those having a carbon number of 3-8 from among the above-mentioned “C₃₋₁₀ cycloalkyl groups”.

The “alkyl group” or the “alkyl group moiety” in the “alkylamino group”, “alkylthio group”, “alkoxy group”, “alkoxycarbonyl group” and the like is a straight chain, branched chain, cyclic or partly cyclic nonaromatic hydrocarbon group and, for example, methyl group, ethyl group, propyl group, isopropyl group, butyl group, isobutyl group, sec-butyl group, tert-butyl group, cyclopropylmethyl group, cyclobutyl group, pentyl group, isopentyl group, neopentyl group, hexyl group, heptyl group, octyl group, nonyl group, decyl group, 1,1-dimethyl-propyl group, cyclopropyl group, cyclopentyl group, cyclohexyl group, cycloheptyl group, cyclooctyl group and the like can be mentioned. Those having a carbon number of 1-10 are preferable, those having a carbon number of 1-6 are more preferable, and those having a carbon number of 1-3 are still more preferable. Particularly preferred are methyl group, ethyl group, isopropyl group, isobutyl group and cyclopropyl group, more preferred are methyl group, ethyl group, isopropyl group and cyclopropyl group.

The “C₁₋₁₀ alkyl group” means those having a carbon number of 1-10 from among the above-mentioned “alkyl groups”, and the “C₁₋₆ alkyl group” means those having a carbon number of 1-6 from among the above-mentioned “alkyl groups”.

The “C₁₋₁₀ alkylthio group” means that wherein the alkyl group moiety is an alkyl group moiety having a carbon number of 1-10 from among the above-mentioned “alkyl group moiety”, specifically, for example, methylthio group, ethylthio group, propylthio group, isopropylthio group, butylthio group, isobutylthio group, sec-butylthio group, tert-butylthio group, cyclopropylmethylthio group, pentylthio group, isopentylthio group, neopentylthio group, hexylthio group, heptylthio group, octylthio group, nonylthio group, decylthio group, 1,1-dimethyl-propylthio group, cyclopropylthio group, cyclobutylthio group, cyclopentylthio group, cyclohexylthio group, cycloheptylthio group, cyclooctylthio group and the like can be mentioned. The “C₁₋₆ alkylthio group” means those having a carbon number of 1-6 from among the above-mentioned “C₁₋₁₀ alkylthio groups”.

The “C₁₋₁₀ alkylamino group” means an amino group mono- or di-substituted by an alkyl group moiety having a carbon number of 1-10 from among the above-mentioned “alkyl group moiety”. Specifically, mono(alkyl)amino groups such as methylamino group, ethylamino group, propylamino group, isopropylamino group, butylamino group, isobutylamino group, sec-butylamino group, tert-butylamino group, cyclopropylmethylamino group, pentylamino group, isopentylamino group, neopentylamino group, hexylamino group, heptylamino group, octylamino group, nonylamino group, decylamino group, (1,1-dimethyl-propyl)amino group, cyclopropylamino group, cyclobutylamino group, cyclopentylamino group, cyclohexylamino group, cycloheptylamino group, cyclooctylamino group and the like; di(alkyl)amino groups such as dimethylamino group, diethylamino group, dipropylamino group, diisopropylamino group, dibutylamino group, diisobutylamino group, di-sec-butylamino group, di-tert-butylamino group, di(cyclopropylmethyl)amino group, dipentylamino group, diisopentylamino group, dineopentylamino group, dihexylamino group, N-methyl-N-ethylamino group, N-methyl-N-propylamino group, N-methyl-N-isopropylamino group, N-methyl-N-butylamino group, N-methyl-N-isobutylamino group, N-methyl-N-sec-butylamino group, N-methyl-N-tert-butylamino group, N-ethyl-N-propylamino group, N-ethyl-N-isopropylamino group, N-ethyl-N-butylamino group, N-ethyl-N-isobutylamino group, N-ethyl-N-sec-butylamino group, N-ethyl-N-tert-butylamino group and the like can be mentioned. The “C₁₋₆ alkylamino group” means those having a carbon number of 1-6 from among the above-mentioned “C₁₋₁₀ alkylamino groups”.

The “C₁₋₁₀ alkoxy group” means that wherein the alkyl group moiety is an alkyl group moiety having a carbon number of 1-10 from among the above-mentioned “alkyl group moiety” and, specifically, methoxy group, ethoxy group, propoxy group, isopropoxy group, butoxy group, isobutoxy group, sec-butoxy group, tert-butoxy group, cyclopropylmethoxy group, pentyloxy group, isopentyloxy group, neopentyloxy group, hexyloxy group, heptyloxy group, octyloxy group, nonyloxy group, decyloxy group, 1,1-dimethyl-propoxy group, cyclopropoxy group, cyclobutoxy group, cyclopentyloxy group, cyclohexyloxy group, cycloheptyloxy group, cyclooctyloxy group and the like can be mentioned. The “C₁₋₆ alkoxy group” means those having a carbon number of 1-6 from among the above-mentioned “C₁₋₁₀ alkoxy groups”.

The “C₂₋₁₀ alkoxycarbonyl group” means that wherein the alkyl group moiety is an alkyl group moiety having a carbon number of 1-9 from among the above-mentioned “alkyl group moiety” and, specifically, methoxycarbonyl group, ethoxycarbonyl group, propoxycarbonyl group, isopropoxycarbonyl group, butoxycarbonyl group, isobutoxycarbonyl group, sec-butoxycarbonyl group, tert-butoxycarbonyl group, cyclopropylmethoxycarbonyl group, pentyloxycarbonyl group, isopentyloxycarbonyl group, neopentyloxycarbonyl group, hexyloxycarbonyl group, heptyloxycarbonyl group, octyloxycarbonyl group, nonyloxycarbonyl group, (1,1-dimethyl-propoxy)carbonyl group, cyclopropoxycarbonyl group, cyclobutoxycarbonyl group, cyclopentyloxycarbonyl group, cyclohexyloxycarbonyl group, cycloheptyloxycarbonyl group, cyclooctyloxycarbonyl group and the like can be mentioned. The “C₂₋₇ alkoxycarbonyl group” means those having a carbon number of 2-7 from among the above-mentioned “C₂₋₁₀ alkoxycarbonyl groups”.

The “alkylamino group” or the “alkylamino group moiety” as a component of the “carbamoyl group substituted by alkyl group” (“a carbamoyl group optionally having substituent(s)” wherein the substituent is an alkyl group), “amidino group substituted by (C₁₋₆) alkyl group”, “guanidino group substituted by (C₁₋₆) alkyl group” and the like includes monoalkylamino group and dialkylamino group. In the dialkylamino group, the two alkyl groups may be the same or different and bonded to each other to optionally formed a ring (e.g., the above-mentioned “nitrogen-containing heterocycle” etc. (e.g., pyrrolidine ring, pyrroline ring)).

As the “halogen atom”, a fluorine atom, a chlorine atom, a bromine atom, an iodine atom and the like can be mentioned, with preference given to a fluorine atom and a chlorine atom.

In the present specification, examples of the substituent when “substituent is present” include

(1) halogen atom,
(2) hydroxyl group,
(3) amino group,
(4) alkyl group having a carbon number of 1-10, preferably 1-6,
(5) alkenyl group having a carbon number of 2-10, preferably 2-6, (e.g., vinyl group, allyl group, isopropenyl group, 1-butenyl group, 2-butenyl group, 3-butenyl group, butadienyl group, 2-methylallyl group, hexatrienyl group, 3-octenyl group etc.),
(6) alkynyl group having a carbon number of 2-10, preferably 2-6, (e.g., ethynyl group, 2-propynyl group, isopropynyl group, butynyl group, tert-butynyl group, 3-hexynyl group etc.),
(7) C₁₋₆ alkoxy group optionally substituted by phenyl,
(8) alkylamino group having a carbon number of 1-6,
(9) cyano group,
(10) guanidino group,
(11) carboxyl group,
(12) carbamoyl group,
(13) aryl group having a carbon number of 6-14, preferably 6-10,
(14) heteroaryl group having a carbon number of 1-10, preferably 1-9,
(15) cycloalkyl group having a carbon number of 3-10, preferably 3-8,
(16) nitrogen-containing nonaromatic heterocyclic group having a carbon number of 1-9, preferably 2-8,
(17) alkylthio group having a carbon number of 1-10, preferably 1-6,
(18) acyloxy group having a carbon number of 1-10, preferably 1-6,
(19) acylamino group having a carbon number of 1-10, preferably 1-6,
(20) alkylsulfonamide group having a carbon number of 1-10, preferably 1-6 (e.g., methylsulfonamide group, ethylsulfonamide group, propylsulfonamide group, isopropylsulfonamide group, butylsulfonamide group, isobutylsulfonamide group, sec-butylsulfonamide group, tert-butylsulfonamide group, cyclopropylmethylsulfonamide group, pentylsulfonamide group, isopentylsulfonamide group, neopentylsulfonamide group, hexylsulfonamide group, heptylsulfonamide group, octylsulfonamide group, nonylsulfonamide group, decylsulfonamide group, (1,1-dimethyl-propyl) sulfonamide group, cyclopropylsulfonamide group, cyclobutylsulfonamide group, cyclopentylsulfonamide group, cyclohexylsulfonamide group, cycloheptylsulfonamide group, cyclooctylsulfonamide group etc.),
(21) alkoxycarbonyl group having a carbon number of 2-10, preferably 2-7, and the like.

The “acyl group” or the “acyl group moiety” as a component of “acyloxy group”, “acylamino group” and the like includes C₁₋₁₁ acyl group such as formyl group, C₂₋₁₀ alkylcarbonyl group (e.g., acetyl group, ethylcarbonyl group, propylcarbonyl group, isopropylcarbonyl group, butylcarbonyl group, isobutylcarbonyl group, sec-butylcarbonyl group, tert-butylcarbonyl group, cyclopropylmethylcarbonyl group, pentylcarbonyl group, isopentylcarbonyl group, neopentylcarbonyl group, hexylcarbonyl group, heptylcarbonyl group, octylcarbonyl group, nonylcarbonyl group, (1,1-dimethyl-propyl)carbonyl group, cyclopropylcarbonyl group, cyclobutylcarbonyl group, cyclopentylcarbonyl group, cyclohexylcarbonyl group, cycloheptylcarbonyl group, cyclooctylcarbonyl group etc.), C₇₋₁₁ arylcarbonyl group (e.g., benzoyl group, 1-naphthylcarbonyl group, 2-naphthylcarbonyl group etc.) and the like. Of these, a C₁₋₁₀ acyl group is preferable, C₁₋₇ acyl group is more preferable, and C₁₋₆ acyl group is particularly preferable.

The “C₁₋₁₀ acyloxy group” means that wherein the acyl group moiety is an acyl group moiety having a carbon number of 1-10 from among the above-mentioned “acyl group moiety” and, specifically, formyloxy group, acetyloxy group, ethylcarbonyloxy group, propylcarbonyloxy group, isopropylcarbonyloxy group, butylcarbonyloxy group, isobutylcarbonyloxy group, sec-butylcarbonyloxy group, tert-butylcarbonyloxy group, cyclopropylmethylcarbonyloxy group, pentylcarbonyloxy group, isopentylcarbonyloxy group, neopentylcarbonyloxy group, hexylcarbonyloxy group, heptylcarbonyloxy group, octylcarbonyloxy group, nonylcarbonyloxy group, (1,1-dimethyl-propyl)carbonyloxy group, cyclopropylcarbonyloxy group, cyclobutylcarbonyloxy group, cyclopentylcarbonyloxy group, cyclohexylcarbonyloxy group, cycloheptylcarbonyloxy group, cyclooctylcarbonyloxy group, benzoyloxy group and the like can be mentioned. The “C₁₋₆ acyloxy group” means those having a carbon number of 1-6 from among the above-mentioned “C₁₋₁₀ acyloxy groups”.

The “C₁₋₁₀ acylamino group” means that wherein the acyl group moiety is an acyl group moiety having a carbon number of 1-10 from among the above-mentioned “acyl group moiety” and, specifically, formylamino group, acetylamino group, ethylcarbonylamino group, propylcarbonylamino group, isopropylcarbonylamino group, butylcarbonylamino group, isobutylcarbonylamino group, sec-butylcarbonylamino group, tert-butylcarbonylamino group, cyclopropylmethylcarbonylamino group, pentylcarbonylamino group, isopentylcarbonylamino group, neopentylcarbonylamino group, hexylcarbonylamino group, heptylcarbonylamino group, octylcarbonylamino group, nonylcarbonylamino group, (1,1-dimethyl-propyl)carbonylamino group, cyclopropylcarbonylamino group, cyclobutylcarbonylamino group, cyclopentylcarbonylamino group, cyclohexylcarbonylamino group, cycloheptylcarbonylamino group, cyclooctylcarbonylamino group, benzoylamino group and the like can be mentioned. The “C₁₋₆ acylamino group” means those having a carbon number of 1-6 from among the above-mentioned “C₁₋₁₀ acylamino groups”.

As the substituent, preferred are

(1) halogen atom,
(2) hydroxyl group,
(3) amino group,
(4) alkyl group having a carbon number of 1-6,
(5) alkenyl group having a carbon number of 2-6,
(6) alkynyl group having a carbon number of 2-6,
(7) C₁₋₆ alkoxy group optionally substituted by phenyl,
(8) alkylamino group having a carbon number of 1-6,
(9) cyano group,
(10) guanidino group,
(11) carboxyl group,
(12) carbamoyl group,
(13) acyloxy group having a carbon number of 1-6,
(14) acylamino group having a carbon number of 1-6,
(15) cycloalkyl group having a carbon number of 3-8,
(16) alkylthio group having a carbon number of 1-6,
(17) alkylsulfonamide group having a carbon number of 1-6, and
(18) alkoxycarbonyl group having a carbon number of 2-10.

As the substituent, more preferred is C₁₋₆ alkoxy group (preferably, methoxy group) optionally substituted by phenyl. Particularly preferred are methoxy group and benzyloxy group.

The number and position of the substituent is not particularly limited.

The compounds represented by the formula (1-1), the formula (1-2) and the formula (1-3) of the present invention (hereinafter sometimes to be abbreviated as compound (1-1), compound (1-2) and compound (1-3)) include a mixture of various stereoisomers such as geometric isomer, tautomer, optical isomer and the like, an isolated form, a stable isotope, and a radioactive isotope.

In the present specification, in the formulas (2-1), (2-2), (3), (3′), (4-1) and (4-2), the bonding sites are shown by *.

In the formula (1-1),

preferred as X is a hydrogen atom or a C₁₋₆ alkyl group optionally having substituent(s) (e.g., hydroxyl group, C₁₋₆ alkoxy group, C₆₋₁₀ aryl ring), more preferably, a methyl group, an ethyl group, a propyl group, an isopropyl group, a butyl group, an isobutyl group and a sec-butyl group, which optionally have a hydroxyl group, a tert-butoxy group, or a phenyl group, and

preferred as Y is a hydrogen atom, a C₁₋₆ alkyl group optionally having substituent(s) or a C₁₋₆ acyl group optionally having substituent(s), more preferably, a methyl group, an ethyl group, a propyl group or an acetyl group.

X and Y are optionally bonded to each other to preferably form a C₂₋₁₀ nitrogen-containing heteroaryl ring or a C₂₋₈ nitrogen-containing nonaromatic heterocycle, which optionally has substituent(s). More preferably, they may form a pyrazole ring, a pyrrolidine ring or a piperidine ring, which optionally has substituent(s) (e.g., a hydroxyl group, a C₁₋₆ alkyl group optionally having substituent(s) (e.g., halogen atom), a C₁₋₆ alkoxy group optionally having substituent(s) (e.g., C₆₋₁₀ aryl ring)). Particularly preferably, they may form a pyrazole ring, a pyrrolidine ring or a piperidine ring, which optionally has a hydroxyl group, a trifluoromethyl group or a benzyloxy group.

Preferred as W is a hydrogen atom, a hydroxyl group, a methoxy group, an ethoxy group, a propoxy group, an isobutoxy group, a 2-hydroxymethoxy group, a cyanomethoxy group, a carboxymethoxy group or a 2-carboxyethyl group, particularly preferably, a hydrogen atom, a hydroxyl group, a 2-hydroxyethoxy group or a cyanomethoxy group.

Y and W are optionally bonded to each other to preferably form a C₂₋₁₀ nitrogen-containing heteroaryl ring or a C₂₋₈ nitrogen-containing nonaromatic heterocycle, which optionally has substituent(s). More preferably, they may form, as a fused ring structure with the adjacent benzene ring, an indole ring, a benzimidazole ring (=benzoimidazole ring), an indazole ring, an indoline ring, a 1,2,3,4-tetrahydroquinoline ring or a 2,3-dihydro-1,3-benzoxazol-2-one ring.

In the formula (2-1), n is preferably 0.

In the formula (2-2), m is preferably 1.

In the formulas (3) and (3′), k is preferably 0 or 1.

Preferred as ring A is a benzene ring, a naphthalene ring, a thiophene ring, a pyridine ring, a piperidine ring or a piperazine ring, particularly preferably a benzene ring, a pyridine ring or a piperidine ring, most preferably a benzene ring.

Preferred as V¹ or V³ is a hydrogen atom, a hydroxyl group, a halogen atom (e.g., a fluorine atom, a chlorine atom), a C₁₋₆ alkyl group (e.g., a methyl group) optionally having substituent(s) (e.g., a hydroxyl group, a C₁₋₆ alkoxy group optionally having substituent(s) (e.g., a C₆₋₁₀ aryl ring), a carboxyl group), a carboxyl group, a carbamoyl group optionally having substituent(s), a C₁₋₆ alkoxy group optionally having substituent(s) (e.g., a methoxy group), or a C₂₋₁₀ alkoxycarbonyl group optionally having substituent(s) (e.g., a methoxycarbonyl group, an ethoxycarbonyl group). Particularly preferred is a hydrogen atom, a hydroxyl group, a fluorine atom, a chlorine atom, a methyl group, a methoxy group, a carboxyl group, a methoxycarbonyl group, an ethoxycarbonyl group, a hydroxymethyl group, a carboxymethyl group, a carbamoyl group, a methoxymethyl group or a benzyloxymethyl group.

In the formula (4-1),

preferred as Z¹ is a single bond, and

preferred as R⁴ is a methyl group, an amino group, a dimethylamino group, a pyrrolidyl group or a pyrrolyl group.

In another embodiment of the present invention, preferred as R⁴ is a C₂₋₈ nitrogen-containing nonaromatic heterocyclic group bonded by nitrogen.

In the formula (4-2), preferred as ring B is a pyridine ring, a pyrrolidine ring, a piperidine ring, a homopiperidine ring, a morpholine ring, a thiomorpholine ring or a piperazine ring, particularly preferably a pyridine ring, a pyrrolidine ring or a piperidine ring.

Preferred as Z² is a single bond, —CO—, an oxygen atom, a sulfur atom or a methylene group, particularly preferably a single bond, —CO— or an oxygen atom.

Preferred as V² is a hydrogen atom, a halogen atom, an amidino group or a C₁₋₆ alkyl group optionally having an imino group at the 1-position, particularly preferably a hydrogen atom, a fluorine atom, a chlorine atom, an amidino group or a 1-iminoethyl group.

Of the compounds represented by the formula (1-1) of the present invention, a compound comprising any combination of preferable groups for each symbol mentioned above is preferable.

In another embodiment of the present invention, a compound represented by the formula (1-2) is preferable from among the compounds represented by the formula (1-1).

In the formula (1-2), preferred as ring C is a pyrazole ring, a pyrrolidine ring, a piperidine ring, a piperazine ring or a thiazolidine ring, particularly preferably a pyrrolidine ring or a piperidine ring.

Preferred as T is a hydrogen atom, a hydroxyl group, a C₁₋₆ alkyl group (e.g., a methyl group) optionally having substituent(s) (e.g., a halogen atom), a C₁₋₁₀ alkoxy group (e.g., a methoxy group, an ethoxy group) optionally having substituent(s) (e.g., a C₆₋₁₀ aryl ring), or a C₁₋₆ carbamoyloxy group optionally having substituent(s) (e.g., a C₁₋₆ alkyl group). Particularly preferred is a hydrogen atom, a hydroxyl group, a trifluoromethyl group, a methoxy group, an ethoxy group, a benzyloxy group or a dimethylcarbamoyloxy group.

In the formula (1-2), preferable examples of each of other symbols are as those exemplified above for the formula (1-1).

In another embodiment of the present invention, a compound represented by the formula (1-3) is preferable from among the compounds represented by the formula (1-1).

In the formula (1-3), preferred as ring D as a fused ring structure with the adjacent benzene ring is an indole ring, a benzimidazole ring (=benzoimidazole ring), an indazole ring, an indoline ring, a 1,2,3,4-tetrahydroquinoline ring or a 2,3-dihydro-1,3-benzoxazol-2-one ring.

In the formula (1-3), preferable examples of each of other symbols are as those exemplified above for the formula (1-1).

More specifically, the compounds described in the Examples are preferable, though not limited thereto.

In the anti-(blood) coagulation drug (agent) for an extracorporeal blood circulation circuit, which contains a low molecular weight FXa inhibitor as an active ingredient, and the method of preventing thrombus formation in an extracorporeal blood circulation circuit, which comprises incorporating a low molecular weight FXa inhibitor as a constituent element of an extracorporeal blood circulation circuit, of the present invention, the low molecular weight FXa inhibitor is a compound represented by the formula (1-1) or a pharmaceutically acceptable salt thereof, or a compound having a molecular weight of 1000 or below and an FXa inhibitory activity, preferably a compound represented by the formula (1-1). As the compound having a molecular weight of 1000 or below and an FXa inhibitory activity, more specifically, for example, the compounds shown in WO99/52895, WO99/10316, WO2000/59876, WO2002/28827, WO96/16940, WO2002/42270 and WO2006/083003 can be mentioned.

As the above-mentioned low molecular weight FXa inhibitor, one that is rapidly cleared from the blood is preferable. Here, being “rapidly cleared from the blood” means not more than 10 min, preferably not more than 5 min, of a half-life in a plasma stability test shown in the below-mentioned Experimental Example 4, more preferably, a decrease of the residual ratio of the above-mentioned low molecular weight FXa inhibitor in a liver S9 stability test (Experimental Example 5) showing the clearance from the body. In addition, as the above-mentioned low molecular weight FXa inhibitor, an FXa selective inhibitor is preferable, more specifically, an inhibitor showing a large difference between pIC₅₀ (FXa) and pIC₅₀ (IIa) in the inhibitory activity evaluation system shown in the below-mentioned Experimental Examples 1 and 2 is preferable.

The extracorporeal blood circulation is an artificial blood circulation via a blood circuit established outside the body, and the extracorporeal blood circulation circuit is a blood circuit in an extracorporeal blood circulation. For example, it is a blood circuit formed by connecting the body and an artificial organ when in use of the artificial organ. More specifically, for example, it is a blood circuit for use of an artificial heart and lung, and hemodialysis. In the present invention, an extracorporeal blood circulation circuit for hemodialysis is particularly preferable.

The representative production method of the compound (1-1) of the present invention is explained in the following.

The formula (1-1) wherein ring A in the formula (3) is a C₆₋₁₀ aryl ring or a C₁₋₁₀ heteroaryl ring, R³ is a group represented by the formula (4-1), and Z¹ is a single bond, amidine derivatives (6) and (8) as intermediates can be obtained by the method shown below. That is, when R¹ is a group represented by the formula (2-1), imidate (5) can be obtained by, for example, dissolving cyanoaryl alcohol such as 4-cyanophenol and the like, or cyanoheteroaryl alcohol in, for example, alcohol such as methanol, ethanol and the like: R⁵OH (R⁵ is an alkyl group) as a solvent and, for example, blowing in a hydrogen chloride gas as an acid. By reacting the thus-obtained imidate (5) with ammonia, an ammonium salt such as ammonium carbonate and the like, or primary or secondary amine: R⁶R⁷NH (wherein R⁶ and R⁷ are the same or different and each is a hydrogen atom or an alkyl group, and R⁶ and R⁷ optionally form, together with the nitrogen atom bonded thereto, a C₂₋₈ nitrogen-containing nonaromatic heterocyclic group) using, for example, alcohol such as methanol, ethanol and the like as a solvent to give amidine derivative (6) of the formula (1-1) wherein R³ is a group represented by the formula (4-1), and Z¹ is a single bond. In addition, when R¹ is a group represented by the formula (2-2), amidine derivative (8) can also be obtained in the same manner by using, for example, cyanoarylcarboxylic acid such as 4-cyanobenzoic acid and the like, or cyanoheteroarylcarboxylic acid.

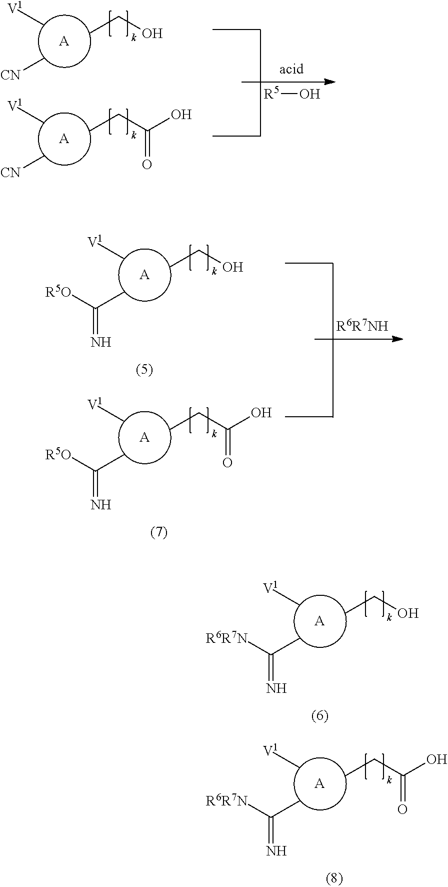

wherein each symbol is as defined above.

When, in the formula (1-1), R¹ is a group represented by the formula (2-1), ring A is a C₆₋₁₀ aryl ring or a C₁₋₁₀ heteroaryl ring, R³ is a group represented by the formula (4-2), ring B is a C₂₋₈ nitrogen-containing nonaromatic heterocycle, Z² is an oxygen atom, and V² is a C₁₋₆ alkyl group optionally having an imino group at the 1-position, an alcohol acid derivative (10) and a carboxylic acid derivative (12) as intermediates can be obtained by the method shown below. That is, when R¹ is a group represented by the formula (2-1), and k is 0, for example, an ether derivative (9) can be obtained by dissolving aryldialcohol or heteroaryldialcohol having two hydroxyl groups, with one of them protected by a suitable protecting group (Prot), which can be removed by a catalytic reduction using palladium, such as 4-benzyloxyphenol and the like, and a nitrogen-containing nonaromatic heterocycle having a hydroxyl group, wherein nitrogen is protected by a suitable protecting group (Prot′), which can be removed under acidic conditions, such as tert-butyl 4-hydroxy-1-piperidinecarboxylate and the like, in a solvent such as tetrahydrofuran (hereinafter THF) and the like, and reacting with diethyl azodicarboxylate (hereinafter DEAD) and triphenylphosphine. The thus-obtained ether derivative (9) is dissolved in a solvent such as ethanol and the like, and subjected to a catalytic reduction using a palladium catalyst such as palladium/carbon and the like under a hydrogen atmosphere, to give an aryl alcohol or heteroaryl alcohol derivative (10) wherein R¹ is a group represented by the formula (2-1) and k is 0. In addition, when R¹ is a group represented by the formula (2-2), ether derivative (13) can be obtained in the same manner as above by using arylcarboxylate or heteroarylcarboxylate having a hydroxyl group, such as ethyl 4-hydroxybenzoate and the like. The thus-obtained ether derivative (13) is hydrolyzed under basic conditions to give carboxylic acid derivative (14) wherein R¹ is a group represented by the formula (2-2). The thus-obtained intermediates (10) and (14) are reacted with an acid such as trifluoroacetic acid, hydrochloric acid/1,4-dioxane solution and the like to give intermediates (11) and (15) without the protecting group on nitrogen. The thus-obtained intermediates (11) and (15) are dissolved in a solvent such as ethanol and the like, and reacted with the corresponding imidate such as ethyl acetimidate and the like in the presence of an organic base such as diisopropylethylamine and the like as necessary to give amidine derivatives (12) and (16).

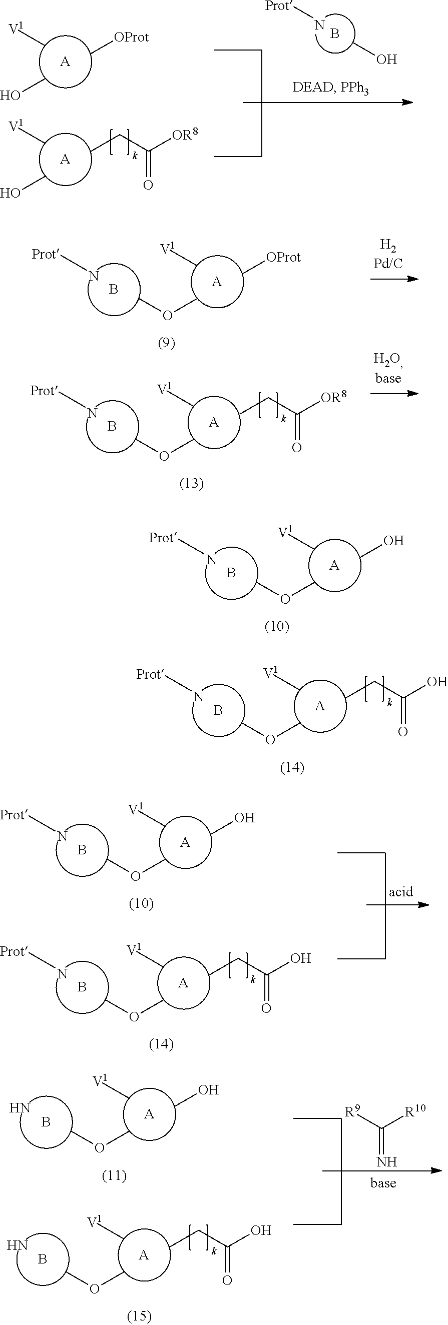

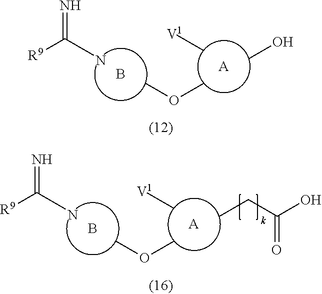

wherein R⁸, R⁹ and R¹⁰ are alkyl groups, and other symbols are as defined above.

When, in the formula (1-1), R¹ is a group represented by the formula (2-1), X is a hydrogen atom, and Y is a C₁₋₁₀ alkyl group optionally having substituent(s), amidic acid derivative (21) as an intermediate can be obtained by the method shown below. That is, by dissolving aminobenzonitrile (e.g., N-(3-cyanophenyl)-2-nitrobenzenesulfonamide and the like) wherein nitrogen is protected by a protecting group (Prot″) such as an o-nitrobenzenesulfonyl group and the like in a solvent such as N,N-dimethylformamide (hereinafter DMF) and the like, and reacting with halogenated acetic acid ester such as tert-butyl bromoacetate and the like in the presence of an inorganic base such as cesium carbonate and the like, ester derivative (17) can be obtained as an intermediate. The thus-obtained intermediate (17) is dissolved in a solvent such as DMF and the like, and reacted with thiol (R′—SH) such as n-dodecylmercaptan or thiophenol and the like in the presence of an inorganic base such as cesium carbonate and the like to give secondary amine derivative (18) without a protecting group on nitrogen as an intermediate. The thus-obtained intermediate (18) is dissolved in a solvent such as DMF and the like, and reacted with the corresponding alkyl halide (Y′-E²) such as methyl iodide and the like in the presence of an inorganic base such as potassium carbonate and the like to give tertiary amine derivative (19) as an intermediate. The thus-obtained intermediate (19) is dissolved in alcohol (R⁵—OH) such as ethanol and the like and, for example, a hydrogen chloride gas is blown in as an acid to give imidate derivative (20) wherein an ester bond is hydrolyzed as an intermediate. The thus-obtained intermediate (20) is dissolved in a solvent such as ethanol and the like, and reacted with, for example, ammonia, an ammonium salt such as ammonium carbonate and the like to give amidine derivative (21).

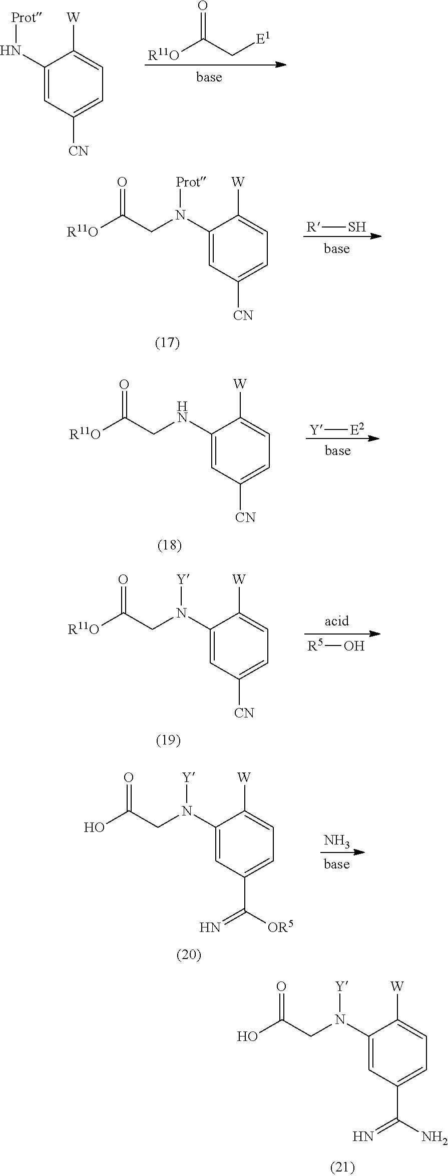

wherein R¹¹ and Y′ are suitable alkyl groups, R′ is a suitable alkyl group or aryl group, E¹ and E² are halogen atoms such as bromine atom, iodine atom and the like, and other symbols are as defined above.

In the formula (1-1), when R¹ is a group represented by the formula (2-1) and Y is a C₁₋₁₀ alkyl group optionally having substituent(s), or X and Y are bonded to each other to form a nitrogen-containing ring, amidine derivative (24) as an intermediate can be obtained by the method shown below. That is, an amino acid such as N-methylalanine and the like is dissolved in a solvent such as DMF, dimethyl sulfoxide and the like, halogenated benzonitrile such as 3-iodobenzonitrile and the like, and a catalytic amount of copper iodide are added and the mixture is heated to give aminobenzonitrile derivative (22) as an intermediate. The thus-obtained intermediate (22) is dissolved in alcohol (R⁵—OH) such as ethanol and the like, and a hydrogen chloride gas is blown in as an acid to give imidate derivative (23) as an intermediate. The thus-obtained intermediate (23) is dissolved in a solvent such as ethanol to and the like, and reacted with ammonia, an ammonium salt such as ammonium carbonate and the like to give amidine derivative (24).

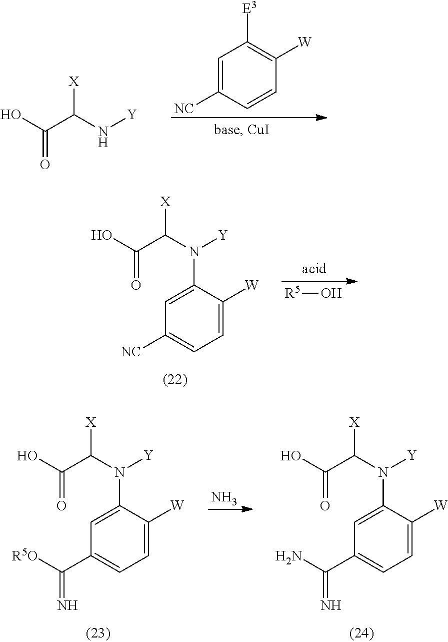

wherein E³ is a halogen atom such as a bromine atom, an iodine atom and the like, and other symbols are as defined above.

When, in the formula (1-1), R¹ is a group represented by the formula (2-2), amidine derivative (27) as an intermediate can be obtained by the method shown below. That is, intermediate (22) is dissolved in a solvent such as THF and the like, reacted with haloformalkyl such as ethyl chloroformate and the like in the presence of an organic base such as triethylamine and the like, and then reacted with a reducing agent such as sodium borohydride and the like to give alcohol derivative (25) as an intermediate. The thus-obtained intermediate (25) is dissolved in alcohol (R⁵—OH) such as ethanol and the like, and a hydrogen chloride gas is blown in as an acid to give imidate derivative (26) as an intermediate. The thus-obtained intermediate (26) is dissolved in a solvent such as ethanol and the like, and reacted with ammonia, an ammonium salt such as ammonium carbonate and the like to give amidine derivative (27).

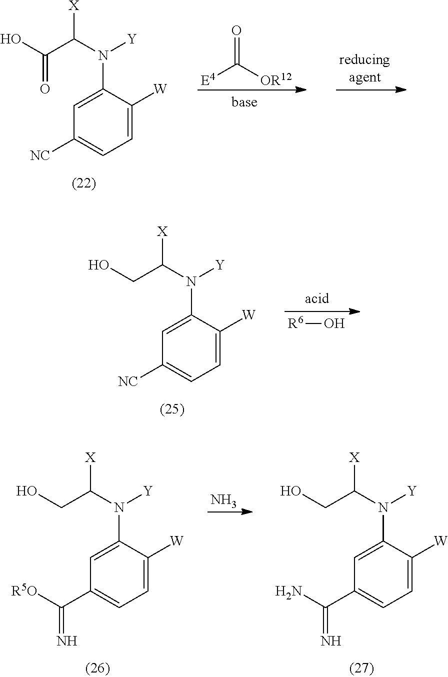

wherein E⁴ is a halogen atom such as a chlorine atom, a bromine atom and the like, R¹² is a suitable alkyl group, and other symbols are as defined above.

When, in the formula (1-1), R¹ is a group represented by the formula (2-1), compound (28) can be obtained by the following method. That is, carboxylic acid intermediate (22) is dissolved in a solvent such as pyridine, N-methylpyrrolidinone (hereinafter NMP) and the like, and reacted with the corresponding alcohol (R²—OH) and a condensing agent such as N,N′-dicyclohexylcarbodiimide (hereinafter DCC), 1-ethyl-3-(3′-dimethylaminopropyl)carbodiimide (hereinafter EDCI) and the like and, where necessary, a catalytic amount of 4-dimethylaminopyridine (hereinafter DMAP) to give compound (28) wherein R¹ is a group represented by the formula (2-1) (n=0). Similarly, when R¹ is a group represented by the formula (2-2), alcohol derivative (27) is dissolved in a solvent such as pyridine, NMP and the like, and reacted with the corresponding carboxylic acid (R²—CO₂H) and a condensing agent such as DCC, EDCI and the like and, where necessary, a catalytic amount of DMAP to give compound (29) wherein R¹ is a group represented by the formula (2-2) (m=1).

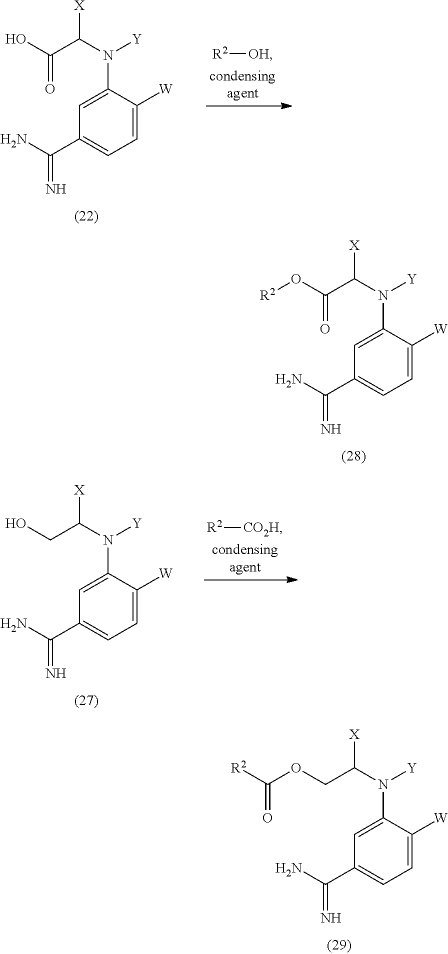

wherein each symbol is as defined above.

When the compound represented by the formula (1-1) of the present invention can form a salt, the salt thereof only needs to be pharmaceutically acceptable and, for example, when an acidic group such as carboxyl group and the like is present in the formula, ammonium salt, salts with alkali metals such as sodium, potassium and the like, salts with alkaline earth metals such as calcium, magnesium and the like, aluminum salt, zinc salt, salts with organic amines such as triethylamine, ethanolamine, morpholine, piperidine, dicyclohexylamine and the like, salts with basic amino acids such as arginine, lysine and the like can be mentioned for the acidic group. When a basic group is present in the formula, salts with inorganic acids such as hydrochloric acid, sulfuric acid, phosphoric acid, nitric acid, hydrobromic acid and the like, salts with organic carboxylic acids such as acetic acid, citric acid, benzoic acid, maleic acid, fumaric acid, tartaric acid, succinic acid, tannic acid, butyric acid, hibenzic acid, pamoic acid, enanthic acid, decanoic acid, teoclic acid, salicylic acid, lactic acid, oxalic acid, mandelic acid, malic acid and the like, salts with organic sulfonic acids such as methanesulfonic acid, benzenesulfonic acid, p-toluenesulfonic acid and the like can be mentioned for the basic group. Examples of the method for forming a salt include mixing of a compound of the formula (1-1), and a necessary acid or base at a suitable quantitative ratio in a solvent or a dispersing agent, and cation exchange or anion exchange of other salt form.

The compound of the present invention includes a solvate, for example, hydrate, alcohol adduct and the like of a compound represented by the formula (1-1).

The compound of the present invention can also be converted to a prodrug. The prodrug in the present invention is a compound that is converted in the body to produce the compound of the present invention. For example, when the active component contains a carboxyl group or a phosphoric acid group, an ester, amide and the like thereof can be mentioned. When the active component contains an amino group, an amide, carbamate and the like thereof can be mentioned. When the active component contains a hydroxyl group, an ester, carbonate, carbamate and the like thereof can be mentioned. When the compound of the present invention is converted to a prodrug, it may be bonded to an amino acid or saccharides.

The amidinoaniline derivative (1) which is the compound of the present invention, or a pharmaceutically acceptable salt thereof can be administered as it is, or administered as a pharmaceutical composition formulated using conventional preparation aids according to a conventional method. Examples of the dosage form of such pharmaceutical composition include tablet, powder, injection, freeze-dry injection, or pill, granule, capsule, suppository, liquid, sugar coating preparation, depot preparation, syrup, suspension, emulsion, troche, hypoglottis, adhesive preparation, orally disintegrant (tablet), inhalant, enteroclysis, ointment, cloth patch preparation, tape preparation, eye drop and the like.

As an administration method of the compound of the present invention or pharmaceutical composition into an extracorporeal blood circulation circuit, or patients, directly administration into an extracorporeal blood circulation circuit, intravenous administration, intramuscular administration, and subcutaneous administration can be preferably mentioned. In some cases, oral administration, intrarectal administration, intranasal administration and sublingual administration are also possible. For direct administration into an extracorporeal blood circulation circuit, administration from a site in a circulation circuit leading the blood out from the body, which site is the nearest to the body, is preferable. In hemodialysis and the like, an injection inlet generally formed in the circuit can be used.

To provide the compound of the present invention or pharmaceutical composition as an anticoagulant for hemodialysis, it may be provided in the form of an FXa inhibitor composition which is dissolved or dispersed in a dialysis solution when in use and used for a dialyzer, or the form of a dialysis solution or dialysis concentrate containing an FXa inhibitor. The dialysis concentrate is diluted into a dialysis solution before use by an appropriate method.

The compound or pharmaceutical composition of the present invention is administered at once or in several portions in a sustained manner as necessary for one operation of extracorporeal blood circulation. The dose of the compound of the present invention or pharmaceutical composition in the amount of an active ingredient compound per one operation of extracorporeal blood circulation or per day is 0.01 mg-10 g, preferably 1 mg-1,000 mg, which can be increased or decreased as appropriate according to the age, body weight, symptom and the like of the patients. While the appropriate concentration of the active ingredient compound in a dialysis solution varies depending on the compound to be used, severity of the disease to be treated and characteristics of the patients under treatment, the appropriate equilibrated average concentration in plasma of the compound, which can be generally used includes a concentration resulting in 0.0001-1000 μmol/L, preferably 0.005-20 μmol/L.

### EXAMPLES

The present invention is explained in detail in the following by referring to Examples, which are not to be construed as limitative.

### Example 1

### 4-[imino(pyrrolidin-1-yl)methyl]phenyl N-{3-[amino(imino)methyl]phenyl}-N-methylglycinate 2 trifluoroacetate

### Step 1 Synthesis of 4-[imino(pyrrolidin-1-yl)methyl]phenol hydrochloride

To a solution (12 mL) of 4-cyanophenol (5.00 g, 42.0 mmol) in dry ethanol was added 4N hydrochloric acid/1,4-dioxane solution (108 mL) and the mixture was stirred under seal at room temperature for 4 days. The solvent was evaporated under reduced pressure and to the obtained residue was added dry ethanol (1000 mL). Pyrrolidine (5.26 mL, 63.0 mmol) was added, and the mixture was stirred at room temperature for 3 days. The solvent was evaporated under reduced pressure and to the obtained residue was added a mixed solvent of ethanol, ethyl acetate and hexane. The mixture was stirred, and the precipitated solid was collected by filtration. To the solid were added 1,4-dioxane (40 mL) and 4N hydrochloric acid/1,4-dioxane solution (12 mL) and the mixture was stirred. The solid was collected by filtration and dried to give the title compound.

yield: 9.03 g (39.8 mmol) yield: 95%

MS (ESI, m/z) 191 [M+H]⁺

¹H-NMR (DMSO-d₆, 400 MHz) δ1.81-1.89 (m, 2H), 2.00-2.07 (m, 2H), 3.46 (t, 2H, J=6.8 Hz), 3.54 (t, 2H, J=6.8 Hz), 6.95-6.98 (m, 2H), 7.46-7.49 (m, 2H), 8.67 (br s, 1H), 9.10 (br s, 1H), 10.52 (br s, 1H).

### Step 2 Synthesis of tert-butyl N-(3-cyanophenyl)-N-[(2-nitrophenyl)sulfonyl]glycinate

To N-(3-cyanophenyl)-2-nitrobenzenesulfonamide (1.7 g, 5.6 mmol) was added cesium carbonate (1.83 g, 5.6 mmol) and the mixture was suspended in N,N-dimethylformamide (hereinafter DMF) (12 mL). tert-Butyl bromoacetate (0.83 mL, 5.6 mmol) was added, and the mixture was stirred at room temperature overnight. Ethyl acetate (50 mL) and 1N hydrochloric acid (50 mL) were added, and the mixture was worked up according to a conventional method, and purified by silica gel column chromatography to give the title compound.

yield: 652 mg (1.56 mmol) yield: 28%

MS (ESI, m/z) 418 [M+H]⁺

### Step 3 Synthesis of tert-butyl N-(3-cyanophenyl)glycinate

The compound (652 mg, 1.56 mmol) obtained in step 2 was dissolved in DMF (6 mL), n-dodecylmercaptan (375 μL, 1.56 mmol) and cesium carbonate (508.8 mg, 1.56 mmol) were added, and the mixture was stirred at 50° C. overnight. After treatment by a conventional method, the obtained residue was subjected to reversed-phase HPLC using octadodecyl group chemically bonded type silica gel (hereinafter ODS) as a filler, eluted with a mixed solution of water and acetonitrile containing trifluoroacetic acid 0.1% (v/v), and the object fraction was lyophilized to give the title compound.

yield: 151 mg (0.65 mmol) yield: 42%

### Step 4 Synthesis of 4-[imino(pyrrolidin-1-yl)methyl]phenyl N-{3-[amino(imino)methyl]phenyl}-N-methylglycinate 2 trifluoroacetate

To the compound (151 mg, 0.65 mmol) obtained in step 3 were added potassium carbonate (179.5 mg, 1.30 mmol) and DMF (2 mL), thereto was added methyl iodide (809 μL, 13.0 mmol), and the mixture was stirred at 60° C. for 2.5 hr. Methyl iodide (405 μL) was appropriately added until the reaction was complete and, after confirmation of the completion of the reaction, and the mixture was worked up according to a conventional method and using ethyl acetate and aqueous citric acid solution. To the obtained residue (151 mg) were added 4N hydrochloric acid/1,4-dioxane solution (4.5 mL) and dry ethanol (0.5 mL), and the mixture was stirred at room temperature for 1 day. The solvent was evaporated under reduced pressure. To the obtained residue was added ammonium carbonate (879 mg, 9.15 mmol), and the mixture was suspended in dry ethanol (10 mL) and stirred overnight. The insoluble material was filtered off and the solvent was evaporated under reduced pressure. To the obtained residue was added 4N hydrochloric acid/ethyl acetate solution. The precipitated solid was collected by filtration, dried under reduced pressure. To the obtained solid (50 mg, about 0.21 mmol) and 4-[imino(pyrrolidin-1-yl)methyl]phenol hydrochloride (46.5 mg, 0.205 mmol) obtained in step 1 were added pyridine (2 mL), DCC (50.8 mg, 0.25 mmol), and a catalytic amount of DMAP, and the mixture was stirred at 50° C. for 30 min. The solvent was evaporated under reduced pressure and the obtained residue was purified by reversed-phase HPLC as in step 3 to give the title compound.

yield: 2.4 mg (0.00395 mmol) yield: 3%

MS (ESI, m/z) 380 [M+H]⁺

¹H-NMR (DMSO-d₆, 300 MHz) δ1.85-1.91 (m, 2H), 2.03-2.07 (m, 2H), 4.66 (s, 2H), 7.09-7.16 (m, 3H), 7.38-7.41 (m, 2H), 7.70-7.73 (m, 2H), 8.75-8.90 (m, 2H), 9.23 (br s, 2H), 9.27 (br s, 1H).

### Example 2

### 4-[imino(pyrrolidin-1-yl)methylphenyl N-acetyl-N-{3-[amino(imino)methyl]phenyl}glycinate 2 trifluoroacetate

60% Sodium hydride (128 mg, 3.19 mmol) was suspended in DMF, and N-(3-cyanophenyl)acetamide (426 mg, 2.66 mmol) which was dissolved in DMF (3 ml) was added under ice-cooling. The mixture was stirred at room temperature for 30 min, ice-cooled again, and tert-butyl bromoacetate (433 μL) was added. The mixture was stirred at 50° C. for 3 hr, the solvent was evaporated, and the residue was worked up using ethyl acetate and 1M aqueous sodium hydroxide solution by a conventional is method to give a crude product (710 mg). To the obtained crude product were added 4N hydrochloric acid/1,4-dioxane solution (18 mL) and ethanol (2 mL), and the mixture was stirred overnight. The solvent was evaporated under reduced pressure, to the obtained crude product were added ammonium carbonate (3.73 g) and ethanol (20 mL) and the mixture was stirred at room temperature for 4 days. The insoluble material was filtered off, the solvent was evaporated, and the obtained residue was purified by reversed-phase HPLC in the same manner as in Example 1, step 3. To the solid obtained by freeze-drying were added 4N hydrochloric acid/1,4-dioxane solution (6 mL) and water (3 mL), and the mixture was stirred at 80° C. for 4 hr. The solvent was evaporated, water was added and the mixture was freeze-dried to give a crude product (66 mg) of N-acetyl-N-{3-[amino(imino)methyl]phenyl}glycine hydrochloride. The obtained crude product and 4-[imino(pyrrolidin-1-yl)methyl]phenol hydrochloride (55.6 mg) obtained in Example 1, step 1 were added pyridine (2 mL), DCC (60.7 mg, 0.29 mmol) and a catalytic amount of DMAP, and the mixture was stirred at 50° C. for 30 min. The solvent was evaporated and the obtained residue was purified by reversed-phase HPLC in the same manner as in Example 1, step 3, to give the title compound.

yield: 3.13 mg (0.00492 mmol) yield: 0.2%

MS (ESI, m/z) 408 [M+H]⁺

### Example 3

### 4-[imino(pyrrolidin-1-yl)methyl]phenyl N-{3-[amino(imino)methyl]phenyl}-N-ethylglycinate 2 trifluoroacetate

The steps similar to those in Example 1 were performed using ethyl iodide instead of methyl iodide to give the title compound.

yield: 6.1 mg (0.000981 mmol)

MS (ESI, m/z) 394 [M+H]⁺

¹H-NMR (DMSO-d₆, 300 MHz) δ1.17 (t, 3H), 1.81-1.89 (m, 2H), 2.00-2.10 (m, 2H), 4.58 (s, 2H), 7.03-7.05 (m, 3H), 7.35-7.40 (m, 2H), 7.70 (d, 2H, J=9.0 Hz), 8.76 (br s, 1H), 8.90 (br s, 2H), 9.25 (br s, 1H).

### Example 4

### 4-[imino(pyrrolidin-1-yl)methyl]phenyl N-{3-[amino(imino)methyl]phenyl}-N-methyl-L-alaninate 2 trifluoroacetate

### Step 1 Synthesis of N-(3-cyanophenyl)-methyl-L-alanine

3-Iodobenzonitrile (916 mg, 4.00 mmol), N-methyl-L-alanine (618 mg, 6.00 mmol), cesium carbonate (1.95 g, 6.00 mmol) and copper iodide (76 mg, 0.4 mmol) were suspended in a mixed solvent of DMF (3.2 mL) and DMSO (0.8 mL). A microwave was irradiated thereon in a tightly-sealed container while stirring at 160° C. for 45 min, and the reaction solution was poured into 1M sodium hydroxide solution (100 mL) and the mixture was washed with dichloromethane. To the aqueous phase was added 3N hydrochloric acid up to about pH3, and the mixture was extracted with dichloromethane. The organic phase was dried over anhydrous magnesium sulfate. The solvent was evaporated under reduced pressure and the obtained residue was purified by silica gel chromatography (dichloromethane:methanol) to give the title compound.

yield: 660 mg (3.23 mmol) yield: 81%

MS (ESI, m/z) 205 [M+H]⁺

¹H-NMR (CDCl₃, 300 MHz) δ1.55 (d, 3H, J=7.2 Hz), 2.93 (s, 3H), 4.51 (q, 1H, J=7.2 Hz), 6.70-7.05 (m, 3H), 7.27-7.33 (m, 1H).

### Step 2 Synthesis of N-{3-[amino(imino)methyl]phenyl}-N-methyl-L-alanine hydrochloride

The compound (657 mg, 3.22 mmol) obtained in step 1 was suspended in 4N hydrochloric acid/1,4-dioxane solution (9 mL) and dry ethanol (1 mL), and the mixture was stirred overnight under seal. The solvent was evaporated under reduced pressure and to the obtained residue were added dry ethanol (10 mL) and ammonium carbonate (1.55 g, 16.1 mmol) and the mixture was stirred at room temperature for 2 days. The solvent was evaporated under reduced pressure and the obtained residue was purified by reversed-phase HPLC in the same manner as in Example 1, step 3 and the obtained solid was suspended in 4N hydrochloric acid/1,4-dioxane solution. The solvent was evaporated and the residue was dissolved in 0.1N hydrochloric acid and freeze-dried to give the title compound.

yield: 296 mg (1.15 mmol) yield: 36%

MS (ESI, m/z) 222 [M+H]⁺

¹H-NMR (DMSO-d₆, 400 MHz) δ1.39 (d, 3H, J=6.8 Hz), 2.85 (s, 3H), 4.76 (q, 1H, J=6.8 Hz), 7.07-7.11 (m, 2H), 7.15-7.16 (m, 1H), 7.38 (dd, 1H, J=8.4 Hz), 9.15 (br s, 2H), 9.32 (br s, 2H).

### Step 3 Synthesis of 4-[imino(pyrrolidin-1-yl)methyl]phenyl N-{3-[amino(imino)methyl]phenyl}-N-methyl-L-alaninate 2 trifluoroacetate

The compound (77.3 mg, 0.300 mmol) obtained in step 2, 4-[imino(pyrrolidin-1-yl)methyl]phenol hydrochloride (68.0 mg, 0.300 mmol) obtained in Example 1, step 1 and DCC (61.9 mg, 0.300 mmol) were suspended in a mixed solvent of pyridine (1.5 mL) and N-methyl-2-pyrrolidinone (hereinafter NMP) (0.5 mL), and the mixture was stirred at 50° C. for 1.5 hr. After concentration under reduced pressure, the residue was purified by reversed-phase HPLC in the same manner as in Example 1, step 3 to give the title compound.

yield: 34.4 mg (0.0553 mmol) yield: 18%

MS (ESI, m/z) 394 [M+H]⁺

¹H-NMR (DMSO-d₆, 400 MHz) δ1.57 (d, 3H, J=6.8 Hz), 1.86 (quint, 2H, J=6.8 Hz), 2.05 (quint, 2H, J=6.8 Hz), 2.97 (s, 3H), 3.37 (t, 2H, J=6.8 Hz), 3.53 (t, 2H, J=6.8 Hz), 5.21 (q, 1H, J=6.8 Hz), 7.13 (d, 1H, J=8 Hz), 7.24-7.26 (m, 2H), 7.35-7.38 (m, 2H), 7.42-7.47 (m, 1H), 7.69-7.72 (m, 2H), 8.80 (br s, 1H), 9.11 (br s, 2H), 9.24 (br s, 2H), 9.29 (br s, 1H).

### Example 5

### 4-[imino(pyrrolidin-1-yl)methyl]phenyl N-{3-[amino(imino)methyl]phenyl}-N-methyl-D-alaninate 2 trifluoroacetate

### Step 1 Synthesis of N-{3-[amino(imino)methyl]phenyl}-N-methyl-D-alanine hydrochloride

The operation similar to that in Example 4, steps 1-2, was performed using N-methyl-D-alanine instead of N-methyl-L-alanine to give the title compound.

yield: 293 mg (1.02 mmol) yield: 24%

MS (ESI, m/z) 222 [M+H]⁺

¹H-NMR (DMSO-d₆, 400 MHz) δ1.39 (d, 3H, J=6.8 Hz), 2.85 (s, 3H), 4.75 (q, 1H, J=6.8 Hz), 7.07-7.11 (m, 2H), 7.14-7.15 (m, 1H), 7.38 (dd, 1H, J=8.0, 7.6 Hz), 9.13 (br s, 2H), 9.31 (br s, 2H).

### Step 2 Synthesis of 4-[imino(pyrrolidin-1-yl)methyl]phenyl N-{3-[amino(imino)methyl]phenyl}-N-methyl-D-alaninate 2 trifluoroacetate

The operation similar to that in Example 4, step 3, was performed using N-{3-[amino(imino)methyl]phenyl}-N-methyl-D-alanine hydrochloride instead of N-{3-[amino(imino)methyl]phenyl}-N-methyl-L-alanine hydrochloride to give the title compound.

MS (ESI, m/z) 394 [M+H]⁺

¹H-NMR (DMSO-d₆, 400 MHz) δ1.57 (d, 3H, J=7.2 Hz), 1.86 (quint, 2H, J=6.8 Hz), 2.05 (quint, 2H, J=6.8 Hz), 2.97 (s, 3H), 3.37 (t, 2H, J=6.8 Hz), 3.53 (t, 2H, J=6.8 Hz), 5.21 (q, 1H, J=7.2 Hz), 7.12-7.14 (m, 1H), 7.24-7.27 (m, 2H), 7.35-7.38 (m, 2H), 7.43-7.47 (m, 1H), 7.69-7.72 (m, 2H), 8.80 (br s, 1H), 9.06 (br s, 2H), 9.24 (br s, 2H), 9.28 (br s, 1H).

### Example 6

### 4-[(1-ethanimidoylpiperidin-4-yl)oxy]phenyl N-{3-[amino(imino)methyl]phenyl}-N-methyl-L-alaninate 2 trifluoroacetate

### Step 1 Synthesis of tert-butyl 4-[4-(benzyloxy)phenoxy]piperidine-1-carboxylate

tert-Butyl 4-hydroxy-1-piperidinecarboxylate (1.51 g, 7.50 mmol), 4-(benzyloxy)phenol (1.50 g, 7.50 mmol) and triphenylphosphine (1.97 g, 7.50 mmol) were dissolved in tetrahydrofuran (25 mL), 40% diethylazodicarboxylic acid-toluene solution (3.27 mL, 7.50 mmol) was added under ice-cooling and the mixture was stirred at room temperature for 6 hr. After concentration under reduced pressure, the residue was worked up by a conventional method, and the obtained crude product was purified by silica gel chromatography (hexane:ethyl acetate) to give the title compound.

yield: 2.00 g (5.22 mmol) yield: 70%

### Step 2 Synthesis of tert-butyl 4-(4-hydroxyphenoxy)piperidine-1-carboxylate

The compound obtained in step 1 (2.00 g, 5.22 mmol) was dissolved in ethanol (25 mL), 10% palladium-carbon (about 200 mg) was added and the mixture was stirred under a hydrogen atmosphere for 4 hr. The reaction solution was filtered through celite, and the solvent was evaporated under reduced pressure to give the title compound without purification.

yield: 1.58 g yield: quantitative

MS (ESI, m/z) 294 [M+H]⁺

### Step 3 Synthesis of 4-[(1-ethanimidoylpiperidin-4-yl)oxy]phenol hydrochloride

The compound (500 mg, 1.70 mmol) obtained in step 2 was dissolved in 1,4-dioxane (5 mL), 4N hydrochloric acid/1,4-dioxane solution (15 ml) was added and the mixture was stirred at room temperature for 2 hr. The solvent was evaporated under reduced pressure and the obtained residue was suspended in dry ethanol (20 mL). Ethyl acetimidate hydrochloride (315 mg, 2.55 mmol) and N,N-diisopropylethylamine (1.18 mL, 6.8 mmol) were added and the mixture was stirred at room temperature overnight. The solvent was evaporated under reduced pressure and the obtained residue was purified by reversed-phase HPLC in the same manner as in Example 1, step 3 and the obtained solid was suspended in 4N hydrochloric acid/1,4-dioxane solution. The solvent was evaporated, and the residue was dissolved in 0.1N hydrochloric acid and freeze-dried to give the title compound.

yield: 345 mg (1.26 mmol) yield: 74%

MS (ESI, m/z) 235 [M+H]⁺

¹H-NMR (DMSO-d₆, 400 MHz) δ1.64-1.77 (m, 2H), 1.94-2.02 (m, 2H), 2.29 (s, 3H), 3.46-3.58 (m, 2H), 3.68-3.82 (m, 2H), 4.45-4.50 (m, 1H), 6.67-6.71 (m, 2H), 6.80-6.84 (m, 2H), 8.78 (br s, 1H), 9.35 (br s, 1H).

### Step 4 Synthesis of 4-[(1-ethanimidoylpiperidin-4-yl)oxy]phenyl N-{3-[amino(imino)methyl]phenyl}-N-methyl-L-alaninate 2 trifluoroacetate

The operation similar to that in Example 4, step 3, was performed using the compound obtained in step 3 instead of 4-[imino(pyrrolidin-1-yl)methyl]phenol hydrochloride to give the title compound.

yield: 93.1 mg (0.140 mmol) yield: 47%

MS (ESI, m/z) 438 [M+H]⁺

¹H-NMR (DMSO-d₆, 400 MHz) δ1.54 (d, 3H, J=7.2 Hz), 1.69-1.80 (m, 2H), 2.01-2.12 (m, 2H), 2.28 (s, 3H), 2.95 (s, 3H), 3.49-3.55 (m, 2H), 3.70-3.79 (m, 2H), 4.65-4.69 (m, 1H), 5.14 (q, 1H, J=7.2 Hz), 7.03 (br s, 4H), 7.11-7.13 (m, 1H), 7.21-7.24 (m, 2H), 7.42-7.46 (m, 1H), 8.59 (br s, 1H), 9.08 (br s, 2H), 9.14 (br s, 1H), 9.24 (br s, 2H).

### Example 7

### 4-[(1-ethanimidoylpiperidin-4-yl)oxy]phenyl N-{3-[amino(imino)methyl]phenyl}-N-methyl-D-alaninate 2 trifluoroacetate

The operation similar to that in Example 6, step 4, was performed using N-{3-[amino(imino)methyl]phenyl}-N-methyl-D-alanine hydrochloride obtained in Example 5, step 1, instead of N-{3-[amino(imino)methyl]phenyl}-N-methyl-L-alanine hydrochloride to give the title compound.

yield: 33.4 mg (0.0502 mmol) yield: 17%

MS (ESI, m/z) 438 [M+H]⁺

¹H-NMR (DMSO-d₆, 400 MHz) δ1.54 (d, 3H, J=6.8 Hz), 1.68-1.80 (m, 2H), 2.00-2.09 (m, 2H), 2.28 (s, 3H), 2.95 (s, 3H), 3.49-3.55 (m, 2H), 3.70-3.78 (m, 2H), 4.64-4.69 (m, 1H), 5.13 (q, 1H, J=6.8 Hz), 7.03 (br s, 4H), 7.10-7.12 (m, 1H), 7.21-7.24 (m, 2H), 7.41-7.46 (m, 1H), 8.59 (br s, 1H), 9.05 (br s, 2H), 9.13 (br s, 1H), 9.23 (br s, 2H).

### Example 8

### 4-[imino(pyrrolidin-1-yl)methyl]phenyl 1-{3-[amino(imino)methyl]phenyl}-L-prolinate 2 trifluoroacetate

### Step 1 Synthesis of 1-(3-cyanophenyl)-L-proline

The operation similar to that in Example 4, step 1, was performed using L-proline instead of N-methyl-L-alanine to give the title compound.

yield: 692 mg (3.20 mmol) yield: 80%

MS (ESI, m/z) 217 [M+H]⁺

### Step 2 Synthesis of 1-{3-[amino(imino)methyl]phenyl}-L-proline hydrochloride

The compound obtained in step 1 (1.18 g, 5.46 mmol) was suspended in 4N hydrochloric acid/1,4-dioxane solution (14 mL) and dry ethanol (1.6 mL), and the mixture was stirred overnight under seal. The solvent was evaporated under reduced pressure and to the obtained residue were added dry ethanol (15 mL) and ammonium carbonate (2.62 g, 27.3 mmol) and the mixture was stirred at room temperature for 2 nights. The solvent was evaporated under reduced pressure and the obtained residue was purified by reversed-phase HPLC in the same manner as in Example 1, step 3. To the obtained solid were added water (4 mL) and 4N hydrochloric acid/1,4-dioxane solution (8 mL), and the mixture was stirred at 80° C. for 3.5 hr. The solvent was evaporated under reduced pressure, and the residue was diluted with water and freeze-dried to give the title compound.

yield: 503 mg (1.86 mmol) yield: 34%

MS (ESI, m/z) 234 [M+H]⁺

### Step 3 Synthesis of 4-[imino(pyrrolidin-1-yl)methyl]phenyl 1-{3-[amino(imino)methyl]phenyl}-L-prolinate 2 trifluoroacetate

The reaction similar to that in Example 4, step 3, was performed using the compound obtained in step 2 instead of N-{3-[amino(imino)methyl]phenyl}-N-methyl-L-alanine hydrochloride, and purification similar to that in Example 1, step 3, was performed by reversed-phase HPLC using ODS and phenyl group-bonded silica gel as column fillers to give the title compound.

yield: 19.3 mg (0.0305 mmol) yield: 8.7%

MS (ESI, m/z) 203 [M+H]²⁺

¹H-NMR (DMSO-d₆, 300 MHz) δ1.81-1.90 (m, 2H), 1.99-2.18 (m, 4H), 2.42-2.49 (m, 2H), 3.56-3.60 (m, 6H), 4.75-4.79 (m, 1H), 6.93-7.00 (m, 2H), 7.10 (d, 1H, J=7.8 Hz), 7.39-7.46 (m, 3H), 7.70-7.73 (m, 2H), 8.81 (br s, 1H), 9.14 (br s, 2H), 9.25 (br s, 2H), 9.29 (br s, 1H).

### Example 9

### 4-[imino(pyrrolidin-1-yl)methyl]phenyl 1-{3-[amino(imino)methyl]phenyl}-D-prolinate 2 trifluoroacetate

### Step 1 Synthesis of 1-{3-[amino(imino)methyl]phenyl}-D-proline hydrochloride

The operation similar to that in Example 8, steps 1 and 2, was performed using D-proline instead of L-proline to give the title compound.

yield: 189 mg (0.701 mmol) yield: 18%

MS (ESI, m/z) 234 [M+H]⁺

### Step 2 Synthesis of 4-[imino(pyrrolidin-1-yl)methyl]phenyl 1-{3-[amino(imino)methyl]phenyl}-D-prolinate 2 trifluoroacetate

The reaction similar to that in Example 4, step 3, was performed using the compound obtained in step 1 instead of N-{3-[amino(imino)methyl]phenyl}-N-methyl-L-alanine hydrochloride, and purification similar to that in Example 1, step 3, was performed by reversed-phase HPLC using phenyl group-bonded silica gel instead of ODS as a column filler to give the title compound.

yield: 31.4 mg (0.0495 mmol) yield: 17%

MS (ESI, m/z) 406 [M+H]⁺

¹H-NMR (DMSO-d₆, 300 MHz) δ1.81-1.90 (m, 2H), 2.01-2.18 (m, 4H), 2.42-2.49 (m, 2H), 3.36-3.60 (m, 6H), 4.75-4.79 (m, 1H), 6.93-7.00 (m, 2H), 7.10 (d, 1H, J=7.8 Hz), 7.39-7.46 (m 3H), 7.70-7.73 (m, 3H), 8.81 (br s, 1H), 9.12 (br s, 2H), 9.25 (br s, 2H), 9.29 (br s, 1H).

### Example 10

### 4-[(1-ethanimidoylpiperidin-4-yl)oxy]phenyl 1-{3-[amino(imino)methyl]phenyl}-L-prolinate 2 trifluoroacetate

### Step 1 Synthesis of tert-butyl 4-{4-[(1-{3-[amino(imino)methyl]phenyl}-L-prolyl)oxy]phenoxy}piperidine-1-carboxylate

The reaction similar to that in Example 8, step 3, was performed using tert-butyl 4-(4-hydroxyphenoxy)piperidine-1-carboxylate obtained in Example 6, step 2, instead of 4-[imino(pyrrolidin-1-yl)methyl]phenol hydrochloride, and purification similar to that in Example 1, step 3, was performed by reversed-phase HPLC using phenyl group-bonded silica gel instead of ODS as a column filler to give the title compound.

yield: 21.3 mg (0.0342 mmol) yield: 9.8%

MS (ESI, m/z) 509 [M+H]⁺

¹H-NMR (DMSO-d₆, 300 MHz) δ1.40 (s, 9H), 1.46-1.55 (m, 2H), 1.83-1.92 (m, 2H), 2.05-2.16 (m, 2H), 2.33-2.45 (m, 2H), 3.11-3.22 (m, 2H), 3.40-3.67 (m, 4H), 4.47-4.55 (m, 1H), 4.66-4.70 (m, 1H), 6.90-7.08 (m, 7H), 7.43 (t, 1H, J=7.8 Hz), 8.89 (br s, 2H), 9.24 (br s, 2H).

### Step 2 Synthesis of 4-[(1-ethanimidoylpiperidin-4-yl)oxy]phenyl 1-{3-[amino(imino)methyl]phenyl}-L-prolinate 2 trifluoroacetate

The operation similar to that in Example 6, step 3, was performed using the compound obtained in step 1 instead of tert-butyl 4-(4-hydroxyphenoxy)piperidine-1-carboxylate to give the title compound.

yield: 15 mg (0.0221 mmol) yield: 65%

MS (ESI, m/z) 225 [M+H]²⁺

¹H-NMR (DMSO-d₆, 300 MHz) δ1.67-1.82 (m, 2H), 1.97-2.16 (m, 4H), 2.28 (s, 3H), 2.34-2.45 (m, 2H), 3.40-3.59 (m, 4H), 3.67-3.80 (m, 2H), 4.63-4.71 (m, 2H), 6.89-6.93 (m, 1H), 6.96-6.99 (m, 1H), 7.04-7.09 (m, 5H), 7.43 (t, 1H, J=7.8 Hz), 8.60 (br s, 1H), 9.10 (br s, 2H), 9.15 (br s, 1H), 9.25 (br s, 2H).

### Example 11

### 4-[(1-ethanimidoylpiperidin-4-yl)oxy]phenyl 1-{3-[amino(imino)methyl]phenyl}-D-prolinate 2 trifluoroacetate

The operation similar to that in Example 9, step 2, was performed using 4-[(1-ethanimidoylpiperidin-4-yl)oxy]phenol hydrochloride obtained in Example 6, step 3 instead of 4-[imino(pyrrolidin-1-yl)methyl]phenol hydrochloride to give the title compound.

yield: 25.7 mg (0.0379 mmol) yield: 65%

MS (ESI, m/z) 225 [M+H]²⁺

¹H-NMR (DMSO-d₆, 300 MHz) δ1.67-1.82 (m, 2H), 1.98-2.16 (m, 4H), 2.28 (s, 3H), 2.34-2.45 (m, 2H), 3.40-3.59 (m, 4H), 3.68-3.80 (m, 2H), 4.63-4.71 (m, 2H), 6.89-6.93 (m, 1H), 6.95-6.99 (m, 1H), 7.01-7.09 (m, 5H), 7.43 (t, 1H, J=7.8 Hz), 8.59 (br s, 1H), 9.07 (br s, 2H), 9.14 (br s, 1H), 9.25 (br s, 2H).

### Example 12

### 4-[imino(pyrrolidin-1-yl)methyl]benzyl 1-{3-[amino(imino)methyl]phenyl}-D-prolinate 2 trifluoroacetate

### Step 1 Synthesis of {4-[imino(pyrrolidin-1-yl)methyl]phenyl}methanol hydrochloride

The operation similar to that in Example 1, step 1, was performed using 4-(hydroxymethyl)benzonitrile instead of 4-cyanophenol, and purification similar to that in Example 1, step 3, was performed by reversed-phase HPLC to give the title compound.

yield: 1.51 g (4.77 mmol) yield: 64%

MS (ESI, m/z) 205 [M+H]⁺

¹H-NMR (DMSO-d₆, 400 MHz) δ1.86 (quint, 2H, J=6.8 Hz), 2.05 (quint, 2H, J=6.8 Hz), 3.40 (t, 2H, J=6.8 Hz), 3.57 (t, 2H, J=6.8 Hz), 4.59 (s, 2H), 7.56 (dd, 4H, J=27, 8.4 Hz), 8.88-8.96 (m, 1H), 9.29 (br s, 1H).

### Step 2 Synthesis of 4-[imino(pyrrolidin-1-yl)methyl]benzyl 1-{3-[amino(imino)methyl]phenyl}-D-prolinate 2 trifluoroacetate

The operation similar to that in Example 9, step 2, was performed using the compound obtained in step 1 instead of 4-[imino(pyrrolidin-1-yl)methyl]phenol hydrochloride to give the title compound.

yield: 13.4 mg (0.0207 mmol) yield: 7%

MS (ESI, m/z) 420 [M+H]⁺

¹H-NMR (DMSO-d₆, 300 MHz) δ1.82-1.89 (m, 2H), 1.98-2.20 (m, 5H), 2.27-2.36 (m, 1H), 3.34-3.44 (m, 3H), 3.49-3.57 (m, 3H), 4.54-4.57 (m, 1H), 5.21-5.30 (m, 2H), 6.79-6.82 (m, 1H), 6.91 (br s, 1H), 7.05 (d, 1H, J=8.1 Hz), 7.37 (t, 1H, J=8.1 Hz), 7.59 (dd, 4H, J=27 Hz, 8.1 Hz), 8.82 (br s, 1H), 9.23-9.27 (m, 5H).

### Example 13

### 4-[imino(pyrrolidin-1-yl)methyl]phenyl (4R)-1-{3-[amino(imino)methyl]phenyl}-4-(benzyloxy)-L-prolinate 2 trifluoroacetate

### Step 1 Synthesis of (4R)-1-{3-[amino(imino)methyl]phenyl}-4(benzyloxy)-L-proline hydrochloride

The operation similar to that in Example 8, steps 1 and 2, was performed using (4R)-4-(benzyloxy)-L-proline instead of L-proline to give the title compound.

yield: 107 mg (0.285 mmol) yield: 9.4%

MS (ESI, m/z) 340 [M+H]⁺

### Step 2 Synthesis of 4-[imino(pyrrolidin-1-yl)methyl]phenyl (4R)-1-{3-[amino(imino)methyl]phenyl}-4-(benzyloxy)-L-prolinate 2 trifluoroacetate

The reaction similar to that in Example 4, step 3, was performed using the compound obtained in step 1 instead of N-{3-[amino(imino)methyl]phenyl}-N-methyl-L-alanine hydrochloride, and purification similar to that in Example 1, step 3, was performed by reversed-phase HPLC using ODS and phenyl group-bonded silica gel as column fillers to give the title compound.

yield: 13.9 mg (0.0187 mmol) yield: 14%

MS (ESI, m/z) 512 [M+H]⁺

¹H-NMR (DMSO-d₆, 300 MHz) δ1.86 (quint, 2H, J=6.9 Hz), 2.05 (quint, 2H, J=6.9 Hz), 2.64-2.68 (m, 2H), 3.38 (t, 2H, J=6.9 Hz), 3.54 (t, 2H, J=6.9 Hz), 3.61-3.66 (m, 1H), 3.77-3.82 (m, 1H), 4.45-4.52 (m, 1H), 4.56-4.65 (m, 2H), 4.88 (t, 1H, J=6.9 Hz), 6.96-6.99 (m, 1H), 7.04 (br s, 1H), 7.13 (d, 1H, J=8.1 Hz), 7.28-7.39 (m, 5H), 7.45 (t, 1H, J=8.1 Hz), 7.70-7.74 (m, 2H), 7.77-7.82 (m, 1H), 8.79-8.84 (m, 2H), 9.25-9.30 (m, 5H).

### Example 14

### 4-[imino(pyrrolidin-1-yl)methyl]benzyl (4R)-1-{3-[amino(imino)methyl]phenyl}-4-(benzyloxy)-L-prolinate 2 trifluoroacetate

The reaction similar to that in Example 13, step 2, was performed using {4-[imino(pyrrolidin-1-yl)methyl]phenyl}methanol hydrochloride obtained in Example 12, step 1, instead of 4-[imino(pyrrolidin-1-yl)methyl]phenol hydrochloride, and purification similar to that in Example 1, step 3, was performed by reversed-phase HPLC using ODS and phenyl group-bonded silica gel as column fillers to give the title compound.

yield: 5.52 mg (0.00732 mmol) yield: 5.4%

MS (ESI, m/z) 526 [M+H]⁺

¹H-NMR (DMSO-d₆, 300 MHz) δ1.87 (quint, 2H, J=6.9 Hz), 2.06 (quint, 2H, J=6.9 Hz), 2.33-2.41 (m, 2H), 3.36 (t, 2H, J=6.9 Hz), 3.52-3.60 (m, 3H), 3.72-3.77 (m, 1H), 4.36-4.44 (m, 1H), 4.51-4.60 (m, 2H), 4.64-4.68 (m, 1H), 5.20-5.30 (m, 2H), 6.80-6.83 (m, 1H), 6.92 (br s, 1H), 7.07 (d, 1H, J=7.8 Hz), 7.29-7.40 (m, 6H), 7.58 (dd, 4H, J=27 Hz, 8.1 Hz), 8.82 (br s, 1H), 9.19-9.26 (m, 5H).

### Example 15

### 4-[imino(pyrrolidin-1-yl)methyl]phenyl (4R)-1-[(3-[amino(imino)methyl]phenyl]-4-hydroxy-L-prolinate 2 trifluoroacetate

To a solution (3 mL) of 4-[imino(pyrrolidin-1-yl)methyl]phenyl (4R)-1-{3-[amino(imino)methyl]phenyl}-4-(benzyloxy)-L-prolinate 2 trifluoroacetate (12.6 mg, 0.0170 mmol) obtained in Example 13 in acetic acid was added a catalytic amount of palladium hydroxide, and the mixture was stirred under a hydrogen atmosphere overnight. The reaction mixture was filtered through celite. The solvent was evaporated under reduced pressure and the obtained residue was diluted with 0.1% aqueous trifluoroacetic acid solution and freeze-dried to give the title compound without purification.

yield: 10.7 mg (0.0165 mmol) yield: 97%

MS (ESI, m/z) 422 [M+H]⁺

### Example 16

### 4-[imino(pyrrolidin-1-yl)methyl]phenyl (2S)-1-{3-[amino(imino)methyl]phenyl}piperidine-2-carboxylate 2 trifluoroacetate

### Step 1 Synthesis of (2S)-1-(3-cyanophenyl)piperidine-2-carboxylic acid

The operation similar to that in Example 4, step 1, was performed using (2S)-piperidine-2-carboxylic acid instead of N-methyl-L-alanine to give the title compound.

yield: 703 mg (3.05 mmol) yield: 76%

MS (ESI, m/z) 231 [M+H]⁺

### Step 2 Synthesis of (2S)-1-{3-[amino(imino)methyl]phenyl}piperidine-2-carboxylic acid hydrochloride

To a solution (15 mL) of the compound obtained in step 1 (703 mg, 3.05 mmol) in methanol were added hydroxylamine hydrochloride (530 mg, 7.63 mmol) and potassium hydroxide (428 mg, 7.63 mmol) and the mixture was stirred at 40° C. overnight. The insoluble material was filtered off, to the filtrate was added acetic anhydride (0.721 mL, 7.63 mmol), and the mixture was stirred at room temperature for 3.5 hr. Acetic anhydride (0.288 mL) was further added and the mixture was stirred at room temperature for 2 hr. A catalytic amount of 10% palladium-carbon was added and the mixture was stirred under a hydrogen atmosphere overnight. The reaction mixture was filtered through celite, concentrated under reduced pressure and the obtained residue was purified by reversed-phase HPLC in the same manner as in Example 1, step 3. The obtained solid was dissolved in 0.1N hydrochloric acid and lyophilized to give the title compound.

yield: 170 mg (0.592 mmol) yield: 19%

MS (ESI, m/z) 248 [M+H]⁺

### Step 3 Synthesis of 4-[imino(pyrrolidin-1-yl)methyl]phenyl (2S)-1-{3-[amino(imino)methyl]phenyl}piperidine-2-carboxylate 2 trifluoroacetate

The operation similar to that in Example 4, step 3, was performed using the compound obtained in step 2 instead of N-{3-[amino(imino)methyl]phenyl}-N-methyl-L-alanine hydrochloride to give the title compound.

yield: 6.9 mg (0.0107 mmol) yield: 3.8%

MS (ESI, m/z) 420 [M+H]⁺

¹H-NMR (DMSO-do 300 MHz) δ1.40-1.70 (m, 2H), 1.76-1.89 (m, 4H), 1.98-2.07 (m, 3H), 2.38-2.43 (m, 1H), 3.09-3.17 (m, 1H), 3.34 (t, 2H, J=6.6 Hz), 3.51 (t, 2H, J=6.6 Hz), 3.73-3.77 (m, 1H), 5.22-5.23 (m, 1H), 7.15-7.18 (m, 1H), 7.27-7.45 (m, 5H), 7.65-7.68 (m, 2H), 8.79 (br s, 1H), 9.17-9.25 (m, 5H).

### Example 17

### 4-[(1-ethanimidoylpiperidin-4-yl)oxy]phenyl (2S)-1-{3-[amino(imino)methyl]phenyl}piperidine-2-carboxylate 2 trifluoroacetate

The operation similar to that in Example 6, step 4, was performed using (2S)-1-{3-[amino(imino)methyl]phenyl}piperidine-2-carboxylic acid hydrochloride obtained in Example 16, step 2, instead of N-{3-[amino(imino)methyl]phenyl}-N-methyl-L-alanine hydrochloride to give the title compound.

yield: 29.3 mg (0.0424 mmol) yield: 9.2%

MS (ESI, m/z) 464 [M+H]⁺

¹H-NMR (DMSO-d₆, 300 MHz) δ1.35-2.10 (m, 9H), 2.26 (s, 3H), 2.33-2.37 (m, 1H), 3.09-3.17 (m, 1H), 3.46-3.53 (m, 2H), 3.68-3.79 (m, 3H), 4.60-4.68 (m, 1H), 5.12-5.15 (m, 1H), 6.92-7.01 (m, 4H), 7.15 (d, 1H, J=7.5 Hz), 7.30-7.33 (m, 2H), 7.41 (t, 1H, J=7.5 Hz), 8.59 (br s, 1H), 9.10-9.22 (m, 5H).

### Example 18

### 4-[imino(pyrrolidin-1-yl)methyl]phenyl (2R)-1-{3-[amino(imino)methyl]phenyl}piperidine-2-carboxylate 2 trifluoroacetate

### Step 1 Synthesis of (2R)-1-{3-[amino(imino)methyl]phenyl}piperidine-2-carboxylic acid hydrochloride

The operation similar to that in Example 16, steps 1 and 2, was performed using (2R)-piperidine-2-carboxylic acid instead of (2S)-piperidine-2-carboxylic acid to give the title compound.

yield: 352 mg (1.24 mmol) yield: 25%

MS (ESI, m/z) 248 [M+H]⁺

¹H-NMR (DMSO-d₆, 400 MHz) δ1.22-1.35 (m, 1H), 1.46-1.58 (m, 1H), 1.64-1.83 (m, 3H), 2.15-2.22 (m, 1H), 3.12 (td, 1H, J=12, 3.2 Hz), 3.64-3.71 (m, 1H), 4.75-4.79 (m, 1H), 7.11-7.13 (m, 1H), 7.19-7.22 (m, 1H), 7.23-7.27 (m, 1H), 7.38 (t, 1H, J=8.0 Hz), 9.02 (br s, 1H), 9.26 (br s, 1H).

### Step 2 Synthesis of 4-[imino(pyrrolidin-1-yl)methyl]phenyl (2R)-1-{3-[amino(imino)methyl]phenyl}piperidine-2-carboxylate 2 trifluoroacetate

The operation similar to that in Example 4, step 3, was performed using the compound obtained in step 1 instead of N-{3-[amino(imino)methyl]phenyl}-N-methyl-L-alanine hydrochloride to give the title compound.

yield: 10.8 mg (0.0166 mmol) yield: 6.1%

MS (ESI, m/z) 420 [M+H]⁺

¹H-NMR (DMSO-d₆, 300 MHz) δ1.40-1.69 (m, 2H), 1.76-1.89 (m, 4H), 1.93-2.09 (m, 3H), 2.38-2.43 (m, 1H), 3.11-3.19 (m, 1H), 3.36 (t, 2H, J=6.6 Hz), 3.53 (t, 2H, J=6.6 Hz), 3.75-3.79 (m, 1H), 5.24-5.25 (m, 1H), 7.18-7.20 (m, 1H), 7.29-7.46 (m, 5H), 7.67-7.70 (m, 2H), 8.81 (br s, 1H), 9.15-9.32 (m, 5H).

### Example 19

### 4-[(1-ethanimidoylpiperidin-4-yl)oxy]phenyl (2R)-1-{(3-[amino(imino)methyl]phenyl}piperidine-2-carboxylate 2 trifluoroacetate

The reaction similar to that in Example 6, step 4, was performed using (2R)-1-{3-[amino(imino)methyl]phenyl}piperidine-2-carboxylic acid hydrochloride obtained in Example 18, step 1, instead of N-{3-[amino(imino)methyl]phenyl}-N-methyl-L-alanine hydrochloride, and purification similar to that in Example 1, step 3, was performed by reversed-phase HPLC using ODS and phenyl group-bonded silica gel as column fillers to give the title compound.

yield: 10.2 mg (0.0147 mmol) yield: 5.3%

MS (ESI, m/z) 464 [M+H]⁺

¹H-NMR (DMSO-d₆, 300 MHz) δ1.35-2.10 (m, 9H), 2.28 (s, 3H), 2.32-2.42 (m, 1H), 3.10-3.19 (m, 1H), 3.48-3.57 (m, 2H), 3.67-3.80 (m, 3H), 4.62-4.69 (m, 1H), 5.11-5.18 (m, 1H), 6.94-7.03 (m, 4H), 7.18 (d, 1H, J=7.5 Hz), 7.31-7.36 (m, 2H), 7.43 (t, 1H, J=7.5 Hz), 8.63 (br s, 1H), 9.17-9.24 (m, 5H).

### Example 20

### 2-chloro-4-[imino(pyrrolidin-1-yl)methyl]phenyl (2R)-1-{3-[amino(imino)methyl]phenyl}piperidine-2-carboxylate 2 trifluoroacetate

### Step 1 Synthesis of 2-chloro-4-[imino(pyrrolidin-1-yl)methyl]phenol hydrochloride

To 4-[imino(pyrrolidin-1-yl)methyl]phenol hydrochloride (664 mg, 2.93 mmol) obtained in Example 1, step 1, were added DMF (10 mL) and N-chlorosuccinimide (hereinafter NCS) (196 mg, 1.47 mmol), and the mixture was stirred at room temperature for 1 hr. NCS (196 mg, 1.47 mmol) was further added, and the mixture was stirred at room temperature overnight. NCS (98 mg, 0.733 mmol) was added twice every 30 min, and the mixture was stirred at 40° C. for 1 hr and at room temperature overnight. The mixture was concentrated under reduced pressure and the obtained residue was purified by reversed-phase HPLC in the same manner as in Example 1, step 3. The obtained solid was dissolved in 0.2N hydrochloric acid and lyophilized to give the title compound.

yield: 71.7 mg (0.276 mmol) yield: 9.4%

MS (ESI, m/z) 225 [M+H]⁺

### Step 2 Synthesis of 2-chloro-4-[imino(pyrrolidin-1-yl)methyl]phenyl (2R)-1-{3-[amino(imino)methyl]phenyl}piperidine-2-carboxylate 2 trifluoroacetate

The operation similar to that in Example 18, step 2, was performed using the compound obtained in step 1 instead of 4-[imino(pyrrolidin-1-yl)methyl]phenol hydrochloride to give the title compound.

yield: 11.9 mg (0.174 mmol) yield: 6.3%

MS (ESI, m/z) 454 [M+H]⁺

¹H-NMR (DMSO-d₆, 300 MHz) δ1.40-1.69 (m, 2H), 1.76-1.89 (m, 4H), 1.94-2.07 (m, 3H), 2.38-2.46 (m, 1H), 3.10-3.21 (m, 1H), 3.35 (t, 2H, J=6.9 Hz), 3.49 (t, 2H, J=6.9 Hz), 3.73-3.83 (m, 1H), 5.28-5.32 (m, 1H), 7.15-7.19 (m, 1H), 7.34-7.49 (m, 4H), 7.64-7.67 (m, 1H), 7.90-7.91 (m, 1H), 8.87 (br s, 1H), 9.09 (br s, 2H), 9.21 (br s, 2H), 9.31 (br s, 1H).

### Example 21

### 2-fluoro-4-[imino(pyrrolidin-1-yl)methyl]phenyl (2R)-1-{3-[amino(imino)methyl]phenyl}piperidine-2-carboxylate 2 trifluoroacetate

### Step 1 Synthesis of 2-fluoro-4-[imino(pyrrolidin-1-yl)methyl]phenol hydrochloride

The reaction similar to that in Example 1, step 1, was performed using 3-fluoro-4-hydroxybenzonitrile instead of 4-cyanophenol, and purification similar to that in Example 1, step 3, was performed by reversed-phase HPLC. The obtained solid was suspended in 4N hydrochloric acid/1,4-dioxane solution, and concentrated under reduced pressure to give the title compound.

yield: 1.34 g (5.48 mmol) yield: 78%

MS (ESI, m/z) 209 [M+H]⁺

¹H-NMR (DMSO-d₆, 400 MHz) δ1.85 (quint, 2H, J=6.8 Hz), 2.03 (quint, 2H, J=6.8 Hz), 3.47 (t, 2H, J=6.8 Hz), 3.52 (t, 2H, J=6.8 Hz), 7.21 (t, 1H, J=8.4, 1.6 Hz), 7.52 (dd, 1H, J=12, 2.0 Hz), 8.78 (br s, 1H), 9.19 (br s, 1H), 11.02 (br s, 1H).

### Step 2 Synthesis of 2-fluoro-4-[imino(pyrrolidin-1-yl)methyl]phenyl (2R)-1-{3-[amino(imino)methyl]phenyl}piperidine-2-carboxylate 2 trifluoroacetate

The operation similar to that in Example 18, step 2, was performed using the compound obtained in step 1 instead of 4-[imino(pyrrolidin-1-yl)methyl]phenol hydrochloride to give the title compound.

yield: 85.2 mg (0.128 mmol) yield: 40%

MS (ESI, m/z) 438 [M+H]⁺

¹H-NMR (DMSO-d₅, 300 MHz) δ1.33-1.50 (m, 1H), 1.57-1.70 (m, 1H), 1.78-1.90 (m, 4H), 1.99-2.09 (m, 3H), 2.36-2.42 (m, 1H), 3.10-3.18 (m, 1H), 3.37 (t, 2H, J=6.9 Hz), 3.52 (t, 2H, J=6.9 Hz), 3.73-3.83 (m, 1H), 5.30-5.35 (m, 1H), 7.18-7.21 (m, 1H), 7.34-7.42 (m, 2H), 7.44-7.55 (m, 3H), 7.74-7.78 (m, 1H), 8.89 (br s, 1H), 9.13 (br s, 2H), 9.23 (br s, 2H), 9.34 (br s, 1H).

### Example 22

### ((2S)-1-{3-[amino(imino)methyl]phenyl}pyrrolidin-2-yl)methyl 4-[imino(pyrrolidin-1-yl)methyl]benzoate 2 trifluoroacetate

### Step 1 Synthesis of 3-[(2S)-2-(hydroxymethyl)pyrrolidin-1-yl]benzonitrile

To a solution (10 mL) of 1-(3-cyanophenyl)-L-proline (692 mg, 3.20 mmol) obtained in Example 8, step 1, in THF were added under ice-cooling triethylamine (0.446 mL, 3.20 mmol) and ethyl chloroformate (0.306 mL, 3.20 mmol), and the mixture was stirred under ice-cooling for 30 min. The insoluble material was filtered off, about 1 g of ice piece and sodium borohydride (121 mg, 3.20 mmol) were added to the filtrate under ice-cooling and the mixture was stirred at room temperature for 1 hr. Under ice-cooling, saturated aqueous ammonium chloride solution (3 mL) was added and the mixture was stirred at room temperature for 30 min, and concentrated under reduced pressure. The obtained residue was worked up by a conventional method, and the obtained crude product was purified by silica gel chromatography (hexane:ethyl acetate) to give the title compound.

yield: 384 mg (1.90 mmol) yield: 59%

MS (ESI, m/z) 203 [M+H]⁺

### Step 2 Synthesis of 3-[(2S)-2-(hydroxymethyl)pyrrolidin-1-yl]benzenecarboximidamide hydrochloride

The operation similar to that in Example 4, step 2, was performed using the compound obtained in step 1 instead of N-(3-cyanophenyl)-methyl-L-alanine to give the title compound.

yield: 339 mg (1.33 mmol) yield: 64%

MS (ESI, m/z) 220 [M+H]⁺

### Step 3 Synthesis of ((2S)-1-{3-[amino(imino)methyl]phenyl}pyrrolidin-2-yl)methyl 4-[imino(pyrrolidin-1-yl)methyl]benzoate 2 trifluoroacetate

The operation similar to that in Example 4, step 3, was performed using the compound obtained in step 2 and 4-[imino(pyrrolidin-1-yl)methyl]benzoic acid hydrochloride instead of 4-[imino(pyrrolidin-1-yl)methyl]phenol hydrochloride to give the title compound.

yield: 25.4 mg (0.0392 mmol) yield: 20%

MS (ESL m/z) 420 [M+H]⁺

¹H-NMR (DMSO-d₆, 300 MHz) δ1.82-1.92 (m, 2H), 1.98-2.21 (m, 6H), 3.15-3.24 (m, 1H), 3.32-3.37 (m, 2H), 3.51-3.59 (m, 3H), 4.20-4.26 (m, 2H), 4.46-4.53 (m, 1H), 7.03-7.10 (m, 3H), 7.41 (t, 1H, J=7.8 Hz), 7.78-7.81 (m, 2H), 8.12-8.16 (m, 2H), 8.98 (br s, 1H), 9.23 (br s, 2H), 9.30 (br s, 2H), 9.41 (br s, 1H).

### Example 23

### ((2R)-1-{3-[amino(imino)methyl]phenyl}pyrrolidin-2-yl)methyl 4-[imino(pyrrolidin-1-yl)methyl]benzoate 2 trifluoroacetate

### Step 1 Synthesis of 3-[(2R)-2-(hydroxymethyl)pyrrolidin-1-yl]benzenecarboximidamide hydrochloride

The operation similar to that in Example 22, steps 1 and 2, was performed using 1-(3-cyanophenyl)-D-proline instead of 1-(3-cyanophenyl)-L-proline to give the title compound.

yield: 360 mg (1.41 mmol) yield: 51%

MS (ESI, m/z) 220 [M+H]⁺

¹H-NMR (DMSO-d₆, 300 MHz) δ1.84-2.07 (m, 4H), 3.06-3.14 (m, 1H), 3.19-3.25 (m, 1H), 3.39-3.53 (m, 2H), 3.75-3.82 (m, 1H), 6.90-6.97 (m, 3H), 7.36 (t, 1H, J=8.1 Hz), 9.04 (br s, 2H), 9.19 (br s, 2H).

### Step 2 Synthesis of ((2R)-1-{3-[amino(imino)methyl]phenyl}pyrrolidin-2-yl)methyl 4-[imino(pyrrolidin-1-yl)methyl]benzoate 2 trifluoroacetate

The operation similar to that in Example 4, step 3, was performed using the compound obtained in step 1 instead of N-{3-[amino(imino)methyl]phenyl}-N-methyl-L-alanine hydrochloride to give the title compound.

yield: 13.2 mg (0.0204 mmol) yield: 10%

MS (ESI, m/z) 420 [M+H]⁺

¹H-NMR (DMSO-d₆, 300 MHz) δ1.83-1.92 (m, 2H), 2.01-2.20 (m, 6H), 3.15-3.26 (m, 1H), 3.32-3.37 (m, 2H), 3.50-3.58 (m, 4H), 4.20-4.25 (m, 2H), 4.46-4.54 (m, 1H), 7.02-7.11 (m, 3H), 7.41 (t, 1H, J=8.1 Hz), 7.78-7.81 (m, 2H), 8.13-8.16 (m, 2H), 8.93 (br s, 1H), 9.07 (br s, 2H), 9.21 (br s, 2H), 9.38 (br s, 1H).

### Example 24

((2S)-1-{3-[amino(imino)methyl]phenyl}pyrrolidin-2-yl)methyl 4-[(1-ethanimidoylpiperidin-4-yl)oxy]benzoate 2 trifluoroacetate

### Step 1 Synthesis of tert-butyl 4-(4-{[((2S)-1-{3-[amino(imino)methyl]phenyl}pyrrolidin-2-yl)methoxy]carbonyl}phenoxy)piperidine-1-carboxylate trifluoroacetate

The operation similar to that in Example 22, step 3, was performed using 4-{[1-(tert-butoxycarbonyl)piperidin-4-yl]oxy}benzoic acid hydrochloride instead of 4-[imino(pyrrolidin-1-yl)methyl]benzoic acid hydrochloride, and EDCI instead of DCC to give the title compound.

yield: 6.1 mg (0.00958 mmol) yield: 1.7%

MS (ESI, m/z) 523 [M+H]⁺

### Step 2 Synthesis of ((2S)-1-{3-[amino(imino)methyl]phenyl}pyrrolidin-2-yl)methyl 4-[(1-ethanimidoylpiperidin-4-yl)oxy]benzoate 2 trifluoroacetate

The operation similar to that in Example 6, step 3, was performed using the compound obtained in step 1 instead of tert-butyl 4-(4-hydroxyphenoxy)piperidine-1-carboxylate to give the title compound.

yield: 6.91 mg (0.00999 mmol) yield: 87%

MS (ESI, m/z) 464 [M+H]⁺

¹H-NMR (DMSO-d₆, 300 MHz) δ1.71-1.87 (m, 2H), 1.98-2.20 (m, 5H), 2.30 (s, 3H), 3.15-3.22 (m, 1H), 3.49-3.59 (m, 3H), 3.71-3.84 (m, 2H), 4.11-4.26 (m, 2H), 4.39-4.45 (m, 1H), 4.81-4.88 (m, 2H), 7.01-7.15 (m, 5H), 7.38-7.43 (m, 1H), 7.90-7.93 (m, 2H), 8.66 (br s, 1H), 9.14-9.31 (m, 5H).

### Example 25

### ((2R)-1-{3-[amino(imino)methyl]phenyl}pyrrolidin-2-yl)methyl 4-[(1-ethanimidoylpiperidin-4-yl)oxy]benzoate 2 trifluoroacetate

The operation similar to that in Example 24, steps 1 and 2, was performed using 3-[(2R)-2-(hydroxymethyl)pyrrolidin-1-yl]benzenecarboximidamide hydrochloride instead of 3-[(2S)-2-(hydroxymethyl)pyrrolidin-1-yl]benzenecarboximidamide hydrochloride to give the title compound.

yield: 9.2 mg (0.0133 mmol) yield: 2.4%

MS (ESI, m/z) 464 [M+H]⁺

¹H-NMR (DMSO-d₆, 300 MHz) δ1.68-1.84 (m, 2H), 1.95-2.18 (m, 5H), 2.27 (s, 3H), 3.21-3.22 (m, 1H), 3.36-3.81 (m, 6H), 4.08-4.22 (m, 2H), 4.38-4.42 (m, 1H), 4.77-4.86 (m, 1H), 6.98-7.12 (m, 5H), 7.35-7.41 (m, 1H), 7.88-7.91 (m, 2H), 8.59 (br s, 1H), 9.00 (br s, 2H), 9.14 (br s, 2H), 9.19 (br s, 2H).

### Example 26

### 4-[imino(pyrrolidin-1-yl)methyl]-2-methoxyphenyl N-{3-[amino(imino)methyl]phenyl}-N-methyl-L-valinate 2 trifluoroacetate

### Step 1 Synthesis of 4-[imino(pyrrolidin-1-yl)methyl]-2-methoxyphenol hydrochloride

To a solution (10 mL) OF 4-hydroxy-3-methoxybenzonitrile (5.00 g, 33.2 mmol) in dry ethanol was added 4N hydrochloric acid/1,4-dioxane solution (40 mL) and the mixture was stirred under seal at room temperature for 2 days. The solvent was evaporated under reduced pressure and to the obtained residue was added dry ethanol (50 mL), pyrrolidine (5.55 mL, 66.6 mmol) was added, and the mixture was stirred at room temperature for 1 day. The solvent was evaporated under reduced pressure and to the obtained residue was added methanol, acetone was added and the mixture was stirred. The precipitated solid was collected by filtration. To the solid were added 1,4-dioxane (40 mL) and 4N hydrochloric acid/1,4-dioxane solution (12 mL) and the mixture was stirred. The solid was collected by filtration and dried to give the title compound.

yield: 3.1 g (12.1 mmol) yield: 36%

MS (ESI, m/z) 221 [M+H]⁺

### Step 2 Synthesis of N-{3-[amino(imino)methyl]phenyl}-N-methyl-L-valine hydrochloride

The operation similar to that in Example 4, steps 1 and 2, was performed using N-methyl-L-valine instead of N-methyl-L-alanine to give the title compound.

yield: 420 mg (1.47 mmol) yield: 24%

MS (ESI, m/z) 250 [M+H]⁺

### Step 3 Synthesis of 4-[imino(pyrrolidin-1-yl)methyl]-2-methoxyphenyl N-{3-[amino(imino)methyl]phenyl}-N-methyl-L-valinate 2 trifluoroacetate

The operation similar to that in Example 4, step 3, was performed using the compound obtained in step 2 instead of N-{3-[amino(imino)methyl]phenyl}-N-methyl-L-alanine hydrochloride and the compound obtained in step 1 instead of 4-[imino(pyrrolidin-1-yl)methyl]phenol hydrochloride to give the title compound.

yield: 37.4 mg (0.0550 mmol) yield: 16%

MS (ESI, m/z) 452 [M+H]⁺

¹H-NMR (DMSO-d₆, 400 MHz) δ0.94 (d, 3H, J=6.6 Hz), 1.14 (d, 3H, J=6.6 Hz), 1.85 (quint, 2H, J=6.8 Hz), 2.05 (quint, 2H, J=6.8 Hz), 2.46-2.34 (m, 1H), 2.96 (s, 3H), 3.40 (t, 2H, J=6.8 Hz), 3.56-3.49 (m, 2H), 3.73 (s, 3H), 4.53 (d, 1H, J=10.4 Hz), 7.15 (d, 1H, J=8.2 Hz), 7.26-7.18 (m, 2H), 7.30-7.26 (m, 1H), 7.41-7.33 (m, 2H), 7.49-7.42 (m, 1H), 8.82 (s, 1H), 9.14 (s, 2H), 9.30-9.24 (m, 3H).

### Example 27

### 4-[imino(pyrrolidin-1-yl)methyl]-2-methoxyphenyl N-{3-[amino(imino)methyl]phenyl}-N-methyl-L-leucinate 2 trifluoroacetate

### Step 1 Synthesis of N-{3-[amino(imino)methyl]phenyl}-N-methyl-L-leucine hydrochloride

The operation similar to that in Example 4, steps 1 and 2, was performed using N-methyl-L-leucine instead of N-methyl-L-alanine to give the title compound.

yield: 375 mg (1.47 mmol) yield: 21%

MS (ESI, m/z) 264 [M+H]⁺

### Step 2 Synthesis of 4-[imino(pyrrolidin-1-yl)methyl]-2-methoxyphenyl N-{3-[amino(imino)methyl]phenyl}-N-methyl-L-leucinate 2 trifluoroacetate

The operation similar to that in Example 26, step 3, was performed using the compound obtained in step 1 instead of N-{3-[amino(imino)methyl]phenyl}-N-methyl-L-valine hydrochloride to give the title compound.

yield: 5.6 mg (0.0087 mmol) yield: 2.3%

MS (ESI, m/z) 466 [M+H]⁺

¹H-NMR (DMSO-d₆, 400 MHz) δ0.90 (d, 3H, J=6.6 Hz), 0.99 (d, 3H, J=6.6 Hz), 1.70-1.55 (m, 1H), 1.94-1.78 (m, 3H), 2.13-1.96 (m, 3H), 3.43-3.35 (m, 2H), 3.57-3.46 (m, 2H), 3.79 (s, 3H), 5.01 (dd, 1H, J=10.2, 5.0 Hz), 7.13 (d, 1H, J=7.6 Hz), 7.25-7.17 (m, 2H), 7.32-7.26 (m, 2H), 7.40 (d, 1H, J=1.9 Hz), 7.45 (t, 1H), 8.84 (s, 1H), 9.14 (s, 2H), 9.39-9.21 (m, 3H).

### Example 28

### 4-[imino(pyrrolidin-1-yl)methyl]phenyl N-{3-[amino(imino)methyl]phenyl}-N-methyl-L-valinate 2 trifluoroacetate

The operation similar to that in Example 4, step 3, was performed using N-{3-[amino(imino)methyl]phenyl}-N-methyl-L-valine hydrochloride instead of N-{3-[amino(imino)methyl]phenyl}-N-methyl-L-alanine hydrochloride to give the title compound.

yield: 22.7 mg (0.0349 mmol) yield: 10%

MS (ESI, m/z) 422 [M+H]⁺

### Example 29

### 4-[imino(pyrrolidin-1-yl)methyl]phenyl N-{3-[amino(imino)methyl]phenyl}-N-methyl-L-leucinate 2 trifluoroacetate

The operation similar to that in Example 4, step 3, was performed using N-{3-[amino(imino)methyl]phenyl}-N-methyl-L-leucine hydrochloride instead of N-{3-[amino(imino)methyl]phenyl}-N-methyl-L-alanine hydrochloride to give the title compound.

yield: 12.8 mg (0.0193 mmol) yield: 5.5%

MS (ESI, m/z) 436 [M+H]⁺

### Example 30

### 4-[(1-ethanimidoylpiperidin-4-yl)oxy]phenyl N-{3-[amino(imino)methyl]phenyl}-N-methyl-L-valinate 2 trifluoroacetate

The operation similar to that in Example 6, step 4, was performed using N-{3-[amino(imino)methyl]phenyl}-N-methyl-L-valine hydrochloride instead of N-{3-[amino(imino)methyl]phenyl}-N-methyl-L-alanine hydrochloride to give the title compound.

yield: 84.5 mg (0.122 mmol) yield: 41%

MS (ESI, m/z) 466 [M+H]⁺

¹H-NMR (DMSO-d₆, 400 MHz) δ0.93 (d, 3H, J=6.6 Hz), 1.11 (d, 3H, J=6.6 Hz), 1.82-1.65 (m, 2H), 2.10-1.96 (m, 2H), 2.28 (s, 3H), 2.47-2.34 (m, 1H), 2.96 (s, 3H), 3.57-3.47 (m, 2H), 3.79-3.67 (m, 2H), 4.48 (d, 1H, J=10.4 Hz), 4.71-4.62 (m, 1H), 6.98-6.91 (m, 2H), 7.04-6.98 (m, 2H), 7.14 (d, 1H, J=7.5 Hz), 7.28-7.22 (m, 1H), 7.37-7.31 (m, 1H), 7.50-7.40 (m, 1H), 8.59 (s, 1H), 9.19-9.06 (m, 3H), 9.26 (s, 2H).

### Example 31

### 4-[(1-ethanimidoylpiperidin-4-yl)oxy]phenyl N-{3-[amino(imino)methyl]phenyl}-N-methyl-L-leucinate 2 trifluoroacetate

The operation similar to that in Example 6, step 4, was performed using N-{3-[amino(imino)methyl]phenyl}-N-methyl-L-leucine hydrochloride instead of N-{3-[amino(imino)methyl]phenyl}-N-methyl-L-alanine hydrochloride to give the title compound.

yield: 123 mg (0.174 mmol) yield: 58%

MS (ESI, m/z) 480 [M+H]⁺

¹H-NMR (DMSO-d₆, 400 MHz) δ0.89 (d, 3H, J=6.6 Hz), 0.97 (d, 3H, J=6.6 Hz), 1.65-1.54 (m, 1H), 1.92-1.66 (m, 3H), 2.10-1.98 (m, 3H), 2.28 (s, 3H), 2.95 (s, 3H), 3.56-3.46 (m, 2H), 3.79-3.67 (m, 2H), 4.70-4.61 (m, 1H), 4.98 (dd, 1H, J=10.3, 5.0 Hz), 7.06-6.97 (m, 4H), 7.11 (d, 1H, J=7.5 Hz), 7.23-7.17 (m, 1H), 7.29 (dd, 1H, J=8.5, 2.3 Hz), 7.43 (t, 1H, J=7.6 Hz), 8.60 (s, 1H), 9.10 (s, 2H), 9.14 (s, 1H), 9.24 (s, 2H).

### Example 32

### 4-[(1-ethanimidoylpiperidin-4-yl)oxy]phenyl N-{3-[amino(imino)methyl]phenyl}-N-methyl-L-isoleucinate 2 trifluoroacetate

### Step 1 Synthesis of N-{3-[amino(imino)methyl]phenyl}-N-methyl-L-isoleucine hydrochloride

The operation similar to that in Example 4, steps 1 and 2, was performed using N-methyl-L-isoleucine instead of N-methyl-L-alanine to give the title compound.

yield: 520 mg (1.73 mmol) yield: 29%

MS (ESI, m/z) 264 [M+H]⁺

### Step 2 4-[(1-ethanimidoylpiperidin-4-yl)oxy]phenyl N-{3-[amino(imino)methyl]phenyl}-N-methyl-L-isoleucinate 2 trifluoroacetate

The operation similar to that in Example 6, step 4, was performed using the compound obtained in step 1 instead of N-{3-[amino(imino)methyl]phenyl}-N-methyl-L-alanine hydrochloride to give the title compound.

yield: 35.1 mg (0.05 mmol) yield: 13%

MS (ESI, m/z) 480 [M+H]⁺

¹H-NMR (DMSO-d₆, 400 MHz) δ0.88 (t, 3H, J=8.0 Hz) 1.07 (d, 1H, J=4.0 Hz), 1.09-1.15 (m, 1H), 1.44-1.51 (m, 1H), 1.67-1.79 (m, 2H), 2.01-2.06 (m, 2H), 2.17-2.25 (m, 1H), 2.28 (s, 3H), 2.96 (s, 3H), 3.48-3.55 (m, 2H), 3.69-3.76 (m, 2H), 4.53 (d, 1H, J=12.0 Hz), 4.64-4.69 (m, 1H), 6.92-7.48 (m, 8H), 8.55-9.26 (m, 6H).

### Example 33

### 4-[imino(pyrrolidin-1-yl)methyl]phenyl N-{3-[amino(imino)methyl]phenyl}-N-methyl-L-isoleucinate 2 trifluoroacetate

The operation similar to that in Example 4, step 3, was performed using N-{3-[amino(imino)methyl]phenyl}-N-methyl-L-isoleucine hydrochloride instead of N-{3-[amino(imino)methyl]phenyl}-N-methyl-L-alanine hydrochloride to give the title compound.

yield: 33.5 mg (0.05 mmol) yield: 16%

MS (ESI, m/z) 436 [M+H]⁺

¹H-NMR (DMSO-d₆, 400 MHz) δ0.89 (t, 3H, J=8.0 Hz), 1.09 (d, 3H, J=8.0 Hz), 1.10-1.15 (m, 1H), 1.45-1.53 (m, 1H), 1.81-1.89 (m, 2H), 2.00-2.09 (m, 2H), 2.19-2.28 (m, 1H), 2.98 (s, 3H), 3.36 (dd, 2H, J=4.0 Hz, 8.0 Hz), 3.52 (t, 2H, J=8.0 Hz), 4.61 (d, 1H, J=8.0 Hz), 7.13-7.49 (m, 6H), 7.66-7.72 (m, 2H), 8.77-9.27 (m, 6H).

### Example 34

### 2-fluoro-4-[imino(pyrrolidin-1-yl)methyl]phenyl N-{3-[amino(imino)methyl]phenyl}-N-methyl-L-valinate 2 trifluoroacetate

The operation similar to that in Example 21, step 2, was performed using N-{3-[amino(imino)methyl]phenyl}-N-methyl-L-valine hydrochloride instead of (2R)-1-{3-[amino(imino)methyl]phenyl}piperidine-2-carboxylic acid hydrochloride to give the title compound.

yield: 131 mg (0.196 mmol) yield: 45%

MS (ESI, m/z) 440 [M+H]⁺

¹H-NMR (DMSO-d₆, 400 MHz) δ0.95 (d, 3H, J=6.6 Hz), 1.13 (d, 3H, J=6.6 Hz), 1.85 (quint, 2H, J=6.8 Hz), 2.04 (quint, 2H, J=6.8 Hz), 2.47-2.39 (m, 1H), 2.96 (s, 3H), 3.37 (t, 2H, J=6.8 Hz), 3.56-3.47 (m, 2H), 4.64 (d, 1H, J=10.4 Hz), 7.16 (d, 1H, J=8.0 Hz), 7.31-7.26 (m, 1H), 7.40-7.34 (m, 1H), 7.55-7.43 (m, 3H), 7.77 (dd, 1H, J=10.4, 1.8 Hz), 8.90 (s, 1H), 9.16 (s, 2H), 9.27 (s, 2H), 9.35 (s, 1H).

### Example 35

### 4-[imino(pyrrolidin-1-yl)methyl]-2-methoxyphenyl N-{3-[amino(imino)methyl]phenyl}-N-methyl-L-isoleucinate 2 trifluoroacetate

The operation similar to that in Example 26, step 3, was performed using N-{3-[amino(imino)methyl]phenyl}-N-methyl-L-isoleucine hydrochloride instead of N-{3-[amino(imino)methyl]phenyl}-N-methyl-L-valine hydrochloride to give the title compound.

yield: 25.0 mg (0.04 mmol) yield: 11%

MS (ESI, m/z) 466 [M+H]⁺

¹H-NMR (DMSO-d₆, 400 MHz) δ0.91 (dd, 3H, J=4.0 Hz, 8.0 Hz), 1.10 (d, 3H, J=4.0 Hz), 1.10-1.16 (m, 1H), 1.47-1.54 (m, 1H), 1.81-1.90 (m, 2H), 2.01-2.10 (m, 2H), 2.17-2.25 (m, 1H), 2.96 (s, 3H), 3.39 (t, 2H, J=8.0 Hz), 3.52 (dd, 2H, J=4.0 Hz, 8.0 Hz), 3.72 (s, 3H), 4.58 (d, 1H, J=8.0 Hz), 7.00-7.50 (m, 7H), 8.61-9.28 (m, 6H).

### Example 36

### 4-[imino(pyrrolidin-1-yl)methyl]-2-methoxyphenyl N-{3-[amino(imino)methyl]phenyl}-N-methyl-L-alaninate 2 trifluoroacetate

The operation similar to that in Example 4, step 3, was performed using 4-[imino(pyrrolidin-1-yl)methyl]-2-methoxyphenol hydrochloride instead of 4-[imino(pyrrolidin-1-yl)methyl]phenol hydrochloride to give the title compound.

yield: 5.0 mg (0.00763 mmol) yield: 2.5%

MS (ESI, m/z) 424 [M+H]⁺

### Example 37

### 4-[imino(pyrrolidin-1-yl)methyl]-2,6-dimethylphenyl N-{3-[amino(imino)methylphenyl]-N-methyl-L-alaninate 2 trifluoroacetate

### Step 1 Synthesis of 2,3-dimethyl-4-[imino(pyrrolidin-1-yl)methyl]-phenol hydrochloride

To a solution (4 mL) of 3,5-dimethyl-4-hydroxybenzonitrile (2.00 g, 13.6 mmol) in dry ethanol was added 4N hydrochloric acid/1,4-dioxane solution (16 mL) and the mixture was stirred under seal at room temperature for 2 days. The solvent was evaporated under reduced pressure and to the obtained residue was added dry ethanol (30 mL), pyrrolidine (2.25 mL, 27.2 mmol) was added, and the mixture was stirred at room temperature for 1 day. The solvent was evaporated under reduced pressure and to the obtained residue was added methanol, acetone was added and the mixture was stirred. The precipitated solid was collected by filtration. To the solid were added 1,4-dioxane (40 mL) and 4N hydrochloric acid/1,4-dioxane solution (12 mL) and the mixture was stirred. The solid was collected by filtration and dried to give the title compound.

yield: 1.1 g (4.3 mmol) yield: 32%

MS (ESI, m/z) 220 [M+H]⁺

### Step 2 Synthesis of 4-[imino(pyrrolidin-1-yl)methyl]-2,6-dimethylphenyl N-{3-[amino(imino)methylphenyl]-N-methyl-L-alaninate 2 trifluoroacetate

The operation similar to that in Example 4, step 3, was performed using the compound obtained in step 1 instead of 4-[imino(pyrrolidin-1-yl)methyl]phenol hydrochloride to give the title compound.

yield: 17.4 mg (0.0268 mmol) yield: 8.6%

MS (ESI, m/z) 422 [M+H]⁺

### Example 38

### 2-fluoro-4-[imino(pyrrolidin-1-yl)methyl]phenyl N-{3-[amino(imino)methyl]phenyl}-N-methyl-L-leucinate 2 trifluoroacetate

The operation similar to that in Example 21, step 2, was performed using N-{3-[amino(imino)methyl]phenyl}-N-methyl-L-leucine hydrochloride instead of (2R)-1-{(3-[amino(imino)methyl]phenyl}piperidine-2-carboxylic acid hydrochloride to give the title compound.

yield: 76.3 mg (0.112 mmol) yield: 42%

MS (ESI, m/z) 454 [M+H]⁺

¹H-NMR (DMSO-d₆, 400 MHz) δ0.90 (d, 3H, J=6.6 Hz), 0.98 (d, 3H, J=6.6 Hz), 1.68-1.57 (m, 1H), 1.91-1.81 (m, 3H), 2.09-2.00 (m, 3H), 2.96 (s, 3H), 3.38 (t, 2H, J=6.8 Hz), 3.55-3.48 (m, 2H), 5.13 (dd, 1H, J=10.3, 4.9 Hz), 7.14 (d, 1H, J=7.6 Hz), 7.23 (br s, 1H), 7.33 (dd, 1H, J=8.5, 2.3 Hz), 7.47-7.41 (m, 1H), 7.57-7.52 (m, 2H), 7.77 (d, 1H, J=11.1 Hz), 8.90 (s, 1H), 9.14 (s, 2H), 9.25 (s, 2H), 9.36 (s, 1H).

### Example 39

### 2-fluoro-4-[imino(pyrrolidin-1-yl)methyl]phenyl N-{3-[amino(imino)methyl]phenyl}-N-methyl-L-isoleucinate 2 trifluoroacetate

The operation similar to that in Example 21, step 2, was performed using N-{3-[amino(imino)methyl]phenyl}-N-methyl-L-isoleucine hydrochloride instead of (2R)-1-{3-[amino(imino)methyl]phenyl}piperidine-2-carboxylic acid hydrochloride to give the title compound.

yield: 76.9 mg (0.113 mmol) yield: 42%

MS (ESI, m/z) 454 [M+H]⁺

¹H-NMR (DMSO-d₆, 400 MHz) δ0.89 (t, 3H, J=7.4 Hz), 1.18-1.06 (m, 4H), 1.55-1.44 (m, 1H), 1.85 (quint, 2H, J=6.8 Hz), 2.04 (quint, 2H, J=6.8 Hz), 2.28-2.18 (m, 1H), 2.96 (s, 3H), 3.37 (t, 2H, J=6.8 Hz), 3.55-3.49 (m, 2H), 4.69 (d, 1H, J=10.5 Hz), 7.19-7.14 (m, 1H), 7.30-7.25 (m, 1H), 7.37 (dd, 1H, J=8.4, 2.2 Hz), 7.55-7.43 (m, 3H), 7.77 (dd, 1H, J=10.4, 1.8 Hz), 8.90 (s, 1H), 9.14 (s, 2H), 9.27 (s, 2H), 9.35 (s, 1H).

### Example 40

### 4-[(1-ethanimidoylpiperidin-4-yl)oxy]phenyl N-{3-[amino(imino)methyl]phenyl}-O-(tert-butyl)-N-methyl-L-serinate 2 trifluoroacetate

### Step 1 Synthesis of N-{3-[amino(imino)methyl]phenyl}-O-(tert-butyl)-N-methyl-L-serine hydrochloride

The operation similar to that in Example 16, steps 1 and 2, was performed using O-(tert-butyl)-N-methyl-L-serine instead of (2S)-piperidine-2-carboxylic acid to give the title compound.

yield: 246 mg (0.746 mmol) yield: 17%

MS (ESI, m/z) 294 [M+H]⁺

### Step 2 Synthesis of 4-[(1-ethanimidoylpiperidin-4-yl)oxy]phenyl N-{3-[amino(imino)methyl]phenyl}-O-(tert-butyl)-N-methyl-L-Serinate 2 trifluoroacetate

The operation similar to that in Example 6, step 4, was performed using the compound obtained in step 1 instead of N-(3-[amino(imino)methyl]phenyl)-N-methyl-L-alanine hydrochloride to give the title compound.

yield: 27.5 mg (0.0373 mmol) yield: 9.8%

MS (ESI, m/z) 510 [M+H]⁺

### Example 41

### 4-[(1-ethanimidoylpiperidin-4-yl)oxy]phenyl N-{3-[amino(imino)methyl]phenyl}-N-methyl-L-norleucinate 2 trifluoroacetate

### Step 1 Synthesis of N-{3-[amino(imino)methyl]phenyl}-N-methyl-L-norleucine hydrochloride

The operation similar to that in Example 4, steps 1 and 2, was performed using N-methyl-L-norleucine instead of N-methyl-L-alanine to give the title compound.

yield: 450 mg (1.500 mmol) yield: 28%

MS (ESI, m/z) 247 [M+H]⁺

### Step 2 Synthesis of 4-[(1-ethanimidoylpiperidin-4-yl)oxy]phenyl N-{3-[amino(imino)methyl]phenyl}-N-methyl-L-norleucinate 2 trifluoroacetate

The operation similar to that in Example 6, step 4, was performed using the compound obtained in step 1 instead of N-{3-[amino(imino)methyl]phenyl}-N-methyl-L-alanine hydrochloride to give the title compound.

yield: 98.7 mg (0.139 mmol) yield: 42%

MS (ESI, m/z) 480 [M+H]⁺

¹H-NMR (DMSO-d₆, 400 MHz) δ0.92-0.83 (m, 3H), 1.41-1.25 (m, 4H), 1.81-1.65 (m, 2H), 2.10-1.94 (m, 4H), 2.28 (s, 3H), 2.94 (s, 3H), 3.55-3.47 (m, 2H), 3.79-3.68 (m, 2H), 4.70-4.63 (m, 1H), 4.96 (dd, 1H, J=9.7, 5.4 Hz), 7.04-6.97 (m, 4H), 7.14-7.08 (m, 1H), 7.22-7.18 (m, 1H), 7.26 (dd, 1H, J=8.4, 2.4 Hz), 7.47-7.38 (m, 1H), 8.60 (s, 1H), 9.10 (s, 2H), 9.14 (s, 1H), 9.24 (s, 2H).

### Example 42

### 4-[(1-ethanimidoylpiperidin-4-yl)oxy]phenyl N-{3-[amino(imino)methyl]phenyl}-N-methyl-L-serinate 2 trifluoroacetate

To 4-[(1-ethanimidoylpiperidin-4-yl)oxy]phenyl N-{3-[amino(imino)methyl]phenyl}-O-(tert-butyl)-N-methyl-L-serinate 2 trifluoroacetate (18 mg, 0.0244 mmol) obtained in Example 40 was added trifluoroacetic acid (1 mL) and the mixture was stirred at room temperature for 5 hr. The mixture was concentrated under reduced pressure and the obtained residue was diluted with water under ice-cooling and lyophilized to give the title compound.

yield: 17.8 mg (0.0261 mmol) yield: quantitative MS (ESI, m/z) 454 [M+H]⁺

¹H-NMR (DMSO-d₆, 400 MHz) δ1.83-1.66 (m, 2H), 2.10-1.97 (m, 2H), 2.28 (s, 3H), 3.04 (s, 3H), 3.64-3.34 (m, 2H), 3.80-3.68 (m, 2H), 4.10-3.98 (m, 2H), 4.72-4.62 (m, 1H), 5.04 (t, 1H, J=6.1 Hz), 7.07-6.98 (m, 4H), 7.13-7.07 (m, 1H), 7.23-7.16 (m, 2H), 7.46-7.39 (m, 1H), 8.59 (br s, 1H), 9.06 (br s, 2H), 9.14 (br s, 1H), 9.24 (br s, 2H).

### Example 43

### 4-[imino(pyrrolidin-1-yl)methyl]-2-methoxyphenyl N-{3-[amino(imino)methyl]phenyl}-N-methyl-L-norleucinate 2 trifluoroacetate

The operation similar to that in Example 26, step 3, was performed using N-{3-[amino(imino)methyl]phenyl}-N-methyl-L-norleucine hydrochloride instead of N-{3-[amino(imino)methyl]phenyl}-N-methyl-L-valine hydrochloride to give the title compound.

yield: 75 mg (0.108 mmol) yield: 32%

MS (ESI, m/z) 466 [M+H]⁺

¹H-NMR (DMSO-d₆, 400 MHz) δ0.93-0.84 (m, 3H), 1.42-1.28 (m, 4H), 1.91-1.80 (m, 2H), 2.13-1.93 (m, 4H), 2.96 (s, 3H), 3.44-3.34 (m, 2H), 3.56-3.48 (m, 2H), 3.79 (s, 3H), 5.00 (dd, 1H, J=9.5, 5.6 Hz), 7.12 (d, 1H, J=7.7 Hz), 7.33-7.19 (m, 4H), 7.47-7.38 (m, 2H), 8.82 (br s, 1H), 9.10 (br s, 2H), 9.33-9.20 (m, 3H).

### Example 44

### 4-[(1-ethanimidoylpiperidin-4-yl)oxy]phenyl N-{3-[amino(imino)methyl]phenyl}-N-methyl-L-norvalinate 2 trifluoroacetate

### Step 1 Synthesis of N-{3-[amino(imino)methyl]phenyl}-N-methyl-L-norvaline hydrochloride

The operation similar to that in Example 16, steps 1 and 2, was performed using N-methyl-L-norvaline instead of (2S)-piperidine-2-carboxylic acid to give the title compound.

yield: 97.9 mg (0.34 mmol) yield: 18%

MS (ESI, m/z) 250 [M+H]⁺

### Step 2 Synthesis of 4-[(1-ethanimidoylpiperidin-4-yl)oxy]phenyl N-{3-[amino(imino)methyl]phenyl}-N-methyl-L-norvalinate 2 trifluoroacetate

The operation similar to that in Example 6, step 4, was performed using the compound obtained in step 1 instead of N-{3-[amino(imino)methyl]phenyl}-N-methyl-L-alanine hydrochloride to give the title compound.

yield: 59.1 mg (0.09 mmol) yield: 25%

MS (ESI, m/z) 467 [M+H]⁺

¹H-NMR (DMSO-d₆, 400 MHz) δ0.93 (dd, 3H, J=8.0 Hz, 4.0 Hz), 1.28-1.44 (m, 2H), 1.67-1.81 (m, 2H), 1.97-2.06 (m, 2H), 2.03-2.08 (m, 2H), 2.28 (s, 3H), 2.94 (s, 3H), 3.49-3.60 (m, 2H), 3.69-3.80 (m, 2H), 4.63-4.69 (m, 1H), 4.98 (dd, 1H, J=12.0 Hz, 4.0 Hz), 6.98-7.46 (m, 8H), 8.60-9.25 (m, 6H).

### Example 45

### 4-[imino(pyrrolidin-1-yl)methyl]-2-methylphenyl N-{3-[amino(imino)methyl]phenyl}-N-methyl-L-norleucinate 2 trifluoroacetate

### Step 1 Synthesis of 4-[imino(pyrrolidin-1-yl)methyl]-2-methylphenol hydrochloride

To a solution of 4-hydroxy-3-methylbenzonitrile (1.53 g, 11.5 mmol) in ethanol (8 mL) was added 4N hydrochloric acid-dioxane (58 mL), and the mixture was stirred at room temperature for 1 day. The reaction mixture was concentrated under reduced pressure to remove ethanol to a certain level, and the solid was collected by filtration. To the obtained solid were added toluene (8 mL) and pyrrolidine (1.9 mL) and the mixture was stirred at room temperature overnight. The reaction mixture was concentrated under reduced pressure, and concentrated hydrochloric acid and water were added and the mixture was stirred with heating. When the solution became uniform, it was concentrated under reduced pressure, and the obtained residue was recrystallized from acetone to give the title compound.

yield: 1.87 g (7.78 mmol) yield: 78%

MS (ESI, m/z) 205 [M+H]⁺

### Step 2 Synthesis of 4-[imino(pyrrolidin-1-yl)methyl]-2-methylphenyl N-{3-[amino(imino)methyl]phenyl}-N-methyl-L-norleucinate 2 trifluoroacetate

The operation similar to that in Example 41, step 2, was performed using the compound obtained in step 1 instead of 4-[(1-ethanimidoylpiperidin-4-yl)oxy]phenol hydrochloride to is give the title compound.

yield: 18.8 mg (0.0277 mmol) yield: 8.7%

MS (ESI, m/z) 450 [M+H]⁺

### Example 46

### 4-[imino(pyrrolidin-1-yl)methyl]phenyl N-{3-[amino(imino)methyl]phenyl}-N-methyl-L-phenylalaninate 2 trifluoroacetate

### Step 1 Synthesis of N-{3-[amino(imino)methyl]phenyl}-N-methyl-L-phenylalanine hydrochloride

The operation similar to that in Example 16, steps 1 and 2, was performed using N-methyl-L-phenylalanine instead of (2S)-piperidine-2-carboxylic acid to give the title compound.

yield: 120.0 mg (0.36 mmol) yield: 10%

MS (ESI, m/z) 298 [M+H]⁺

### Step 2 Synthesis of 4-[imino(pyrrolidin-1-yl)methyl]phenyl N-{3-[amino(imino)methyl]phenyl}-N-methyl-L-phenylalaninate 2 trifluoroacetate

The operation similar to that in Example 4, step 3, was performed using N-{3-[amino(imino)methyl]phenyl}-N-methyl-L-phenylalanine hydrochloride instead of N-{3-[amino(imino)methyl]phenyl}-N-methyl-L-alanine hydrochloride to give the title compound.

yield: 37.8 mg (0.05 mmol) yield: 15%

MS (ESI, m/z) 470 [M+H]

¹H-NMR (DMSO-d₅, 400 MHz) δ1.81-1.90 (m, 2H), 2.01-2.09 (m, 2H), 2.99 (s, 3H), 3.30-3.34 (m, 1H), 3.37 (dd, 2H, J=8.0 Hz, 4.0 Hz), 3.53 (t, 2H, J=8.0 Hz), 5.41-5.47 (m, 1H), 7.04-7.39 (m, 11H), 7.68-7.74 (m, 2H), 8.78-9.30 (m, 6H).

### Example 47

### 4-[imino(pyrrolidin-1-yl)methyl]phenyl 2-[{3-[amino(imino)methyl]phenyl}(methyl)amino]butanoate 2 trifluoroacetate

### Step 1 Synthesis of 2-[{3-[amino(imino)methyl]phenyl}(methyl)amino]butanoic acid hydrochloride

The operation similar to that in Example 4, steps 1 and 2, was performed using 2-(methylamino)butanoic acid instead of N-methyl-L-alanine to give the title compound.

yield: 330 mg (0.95 mmol) yield: 11%

MS (ESI, m/z) 236 [M+H]⁺

### Step 2 Synthesis of 4-[imino(pyrrolidin-1-yl)methyl]phenyl 2-[{3-[amino(imino)methyl]phenyl}(methyl)amino]butanoate 2 trifluoroacetate

The operation similar to that in Example 4, step 3, was performed using the compound obtained in step 1 instead of N-{3-[amino(imino)methyl]phenyl}-N-methyl-L-alanine hydrochloride to give the title compound.

yield: 59.6 mg (0.09 mmol) yield: 26%

MS (ESI, m/z) 408 [M+H]⁺

¹H-NMR (DMSO-d₅, 400 MHz) δ0.96 (t, 2H, J=8.0 Hz), 1.81-1.92 (m, 2H), 1.96-2.10 (m, 3H), 2.10-2.20 (m, 1H), 2.96 (s, 3H), 3.37 (dd, 2H, J=8.0 Hz, 4.0 Hz), 3.53 (dd, 2H, J=8.0 Hz, 4.0 Hz), 4.96-5.02 (m, 1H), 7.10-7.47 (m, 6H), 7.67-7.73 (m, 2H), 8.78-9.29 (m, 6H).

### Example 48

### 2-fluoro-4-[imino(pyrrolidin-1-yl)methyl]phenyl 2-[{3-[amino(imino)methyl]phenyl}(methyl)amino]butanoate 2 trifluoroacetate

The operation similar to that in Example 21, step 2, was performed using 2-[{3-[amino(imino)methyl]phenyl}(methyl)amino]butanoic acid hydrochloride instead of (2R)-1-{(3-[amino(imino)methyl]phenyl}piperidine-2-carboxylic acid hydrochloride to give the title compound.

yield: 34.2 mg (0.05 mmol) yield: 14%

MS (ESI, m/z) 426 [M+H]⁺

¹H-NMR (DMSO-d₆, 400 MHz) δ0.97 (t, 3H, J=8.0 Hz), 1.81-1.90 (m, 2H), 1.98-2.09 (m, 3H), 2.09-2.20 (m, 1H), 2.95 (s, 3H), 3.38 (t, 2H, J=8.0 Hz), 3.52 (t, 2H, J=8.0 Hz), 5.06-5.11 (m, 1H), 7.12-7.58 (m, 6H), 7.75-7.80 (m, 1H), 8.91-9.38 (m, 6H).

### Example 49

### 4-[imino(pyrrolidin-1-yl)methyl]-2,6-dimethylphenyl (2R)-1-{3-[amino(imino)methyl]phenyl}piperidine-2-carboxylate 2 trifluoroacetate

The operation similar to that in Example 18, step 2, was performed using 2,3-dimethyl-4-[imino(pyrrolidin-1-yl)methyl]-phenol hydrochloride instead of the synthesis of 4-[imino(pyrrolidin-1-yl)methyl]phenol hydrochloride to give the title compound.

yield: 13.5 mg (0.0200 mmol) yield: 5.7%

MS (ESI, m/z) 448 [M+H]⁺

¹H-NMR (DMSO-d₆, 400 MHz) δ1.34-1.50 (m, 1H), 1.57-1.71 (m, 1H), 1.79-1.94 (m, 3H), 1.95-2.22 (m, 8H), 2.39-2.48 (m, 1H), 3.19 (ddd, 1H, J=12 Hz, 12 Hz, 2.5 Hz), 3.33-3.41 (m, 2H), 3.46-3s 3.54 (m, 2H), 3.71-3.79 (m, 1H), 5.41 (dd, 1H, J=5.9 Hz, 2.0 Hz), 7.22 (d, 1H, J=7.1 Hz), 7.36-7.49 (m, 5H), 8.77 (s, 1H), 9.18-9.32 (m, 5H).

### Example 50

### 6-[imino(pyrrolidin-1-yl)methyl]pyridin-3-yl (2R)-1-{3-[amino(imino)methyl]phenyl}piperidine-2-carboxylate 2 trifluoroacetate

### Step 1 Synthesis of 6-[imino(pyrrolidin-1-yl)methyl]pyridin-3-ol hydrochloride

To 5-hydroxypyridine-2-carbonitrile (1.00 g, 8.33 mmol) was added methanol (13.0 mL), and L-N-acetylcysteine (1.43 g, 8.76 mmol) and pyrrolidine (2.1 mL, 25.6 mmol) were added and the mixture was stirred at 60° C. for 2 hr and at room temperature for 1 day. The reaction mixture was concentrated under reduced pressure and the obtained residue was purified by reversed-phase HPLC in the same manner as in Example 1, step 3. To the obtained solid were added concentrated hydrochloric acid and water and the mixture was concentrated under reduced pressure and the residue was recrystallized from methanol, acetone and toluene to give the title compound.

yield: 0.80 g (3.54 mmol) yield: 43%

MS (ESI, m/z) 192 [M+H]⁺

### Step 2 Synthesis of 6-[imino(pyrrolidin-1-yl)methyl]pyridin-3-yl (2R)-1-{3-[amino(imino)methyl]phenyl}piperidine-2-carboxylate 2 trifluoroacetate

The operation similar to that in Example 18, step 2, was performed using the compound obtained in step 1 instead of 4-[imino(pyrrolidin-1-yl)methyl]phenol hydrochloride to give the title compound.

yield: 29.4 mg (0.0453 mmol) yield: 13%

MS (ESI, m/z) 421 [M+H]⁺

¹H-NMR (DMSO-d₆, 400 MHz) δ1.40-1.54 (m, 1H), 1.55-1.68 (m, 1H), 1.76-1.93 (m, 4H), 1.94-2.11 (m, 3H), 2.39-2.48 (m, 1H), 3.07-3.20 (m, 1H), 3.28-3.68 (m, 4H), 3.72-3.82 (m, 1H), 5.24-5.31 (m, 1H), 7.18 (d, 1H, J=7.6 Hz), 7.33-7.39 (m, 2H), 7.44 (dd, 1H, J=9.1 Hz, 7.6 Hz), 7.91 (d, 1H, J=0.7 Hz), 7.92 (d, 1H, J=2.5 Hz), 8.58 (dd, 1H, J=2.5 Hz, 0.7 Hz), 8.95 (s, 2H), 9.00 (s, 1H), 9.23 (s, 2H), 9.45 (s, 1H).

### Example 51

### 4-[imino(pyrrolidin-1-yl)methyl]-2-(methoxycarbonyl)phenyl (2R)-1-{3-[amino(imino)methyl]phenyl}piperidine-2-carboxylate 2 trifluoroacetate

### Step 1 Synthesis of 5-cyano-2-hydroxybenzoic acid

To formic acid (25 ml) were added 5-formylsalicylic acid (2.0 g, 12.0 mmol), sodium formate (1.56 g, 22.9 mmol) and hydroxylamine sulfate (1.19 g, 7.22 mmol). The solution was stirred at 80° C. for 6 hr, and cooled to room temperature. After dilution with ethyl acetate, the organic layer was washed with water and saturated brine, dried over anhydrous magnesium sulfate and the solvent was removed under reduced pressure to give the title compound without purification.

yield: 1.95 g (11.9 mmol) yield: 99%

MS (ESI, m/z) 164 [M+H]⁺

### Step 2 Synthesis of 2-hydroxy-5-[imino(pyrrolidin-1-yl)methyl]benzoic acid trifluoroacetate

The reaction similar to that in Example 1, step 1, was performed using the compound obtained in step 1 instead of 4-hydroxybenzonitrile, and purification similar to that in Example 1, step 3, was performed by reversed-phase HPLC to give the title compound.

yield: 716 mg (1.33 mmol) yield: 35%

MS (ESI, m/z) 235 [M+H]⁺

¹H-NMR (DMSO-d₆, 400 MHz) δ1.81-1.90 (m, 2H), 1.99-2.09 (m, 2H), 3.40-3.47 (m, 2H), 3.48-3.55 (m, 2H), 7.11 (d, 1H, J=8.6 Hz), 7.73 (dd, 1H, J=8.6 Hz, 2.2 Hz), 8.05 (d, 1H, J=2.2 Hz), 8.69 (s, 1H), 9.19 (s, 1H).

### Step 3 Synthesis of methyl 2-hydroxy-5-[imino(pyrrolidin-1-yl)methyl]benzoate trifluoroacetate

The compound obtained in step 2 (716 mg, 2.06 mmol) was dissolved in methanol (10 mL), 2N diazomethane/diethyl ether solution (2.0 ml, 4.00 mol) was added, and the mixture was stirred at room temperature for 1 hr. The solvent was removed under reduced pressure, and the obtained residue was purified by reversed-phase HPLC in the same manner as in Example 1, step 3, to give the title compound.

yield: 196 mg (0.541 mmol) yield: 26%

MS (ESI, m/z) 249 [M+H]⁺

¹H-NMR (DMSO-d₆, 400 MHz) δ1.80-1.91 (m, 2H), 1.96-2.09 (m, 2H), 2.29 (s, 1H), 3.38-3.46 (m, 2H), 3.49-3.56 (m, 2H), 3.90 (s, 3H), 7.19 (d, 1H, J=8.3 Hz), 7.75 (dd, 1H, J=8.3 Hz, 2.2 Hz), 8.02 (d, 1H, J=2.2 Hz), 8.75 (s, 1H), 9.22 (s, 1H).

### Step 4 Synthesis of 4-[imino(pyrrolidin-1-yl)methyl]-2-(methoxycarbonyl)phenyl (2R)-1-{3-[amino(imino)methyl]phenyl}piperidine-2-carboxylate 2 trifluoroacetate

The operation similar to that in Example 18, step 2, was performed using the compound obtained in step 3 instead of the synthesis of 4-[imino(pyrrolidin-1-yl)methyl]phenol hydrochloride to give the title compound.

yield: 61.6 mg (0.0873 mmol) yield: 33%

MS (ESI, m/z) 478 [M+H]⁺

¹H-NMR (DMSO-d₆, 400 MHz) δ1.41-1.70 (m, 2H), 1.77-1.90 (m, 4H), 1.92-2.10 (m, 3H), 2.42-2.56 (m, 1H), 3.09-3.21 (m, 1H), 3.30-3.70 (m, 5H), 3.77 (s, 3H), 5.22-5.27 (m, 1H), 7.16 (d, 1H, J=7.6 Hz), 7.32 (s, 1H), 7.40 (d, 1H, J=8.3 Hz), 7.43 (d, 1H, J=7.6 Hz), 7.92 (dd, 1H, J=8.3 Hz, 2.2 Hz), 8.15 (d, 1H, J=2.2 Hz), 8.85 (s, 1H), 8.96 (s, 1H), 9.22 (s, 2H), 9.32 (s, 1H).

### Example 52

### 2-{[((2R)-1-{3-[amino(imino)methyl]phenyl}piperidin-2-yl)carbonyl]oxy}-5-[imino(pyrrolidin-1-yl)methyl]benzoic acid 2 trifluoroacetate

The operation similar to that in Example 18, step 2, was performed using 2-hydroxy-5-[imino(pyrrolidin-1-yl)methyl]benzoic acid trifluoroacetate instead of the synthesis of 4-[imino(pyrrolidin-1-yl)methyl]phenol hydrochloride to give the title compound.

yield: 17.0 mg (0.0873 mmol) yield: 17%

MS (ESI, m/z) 464 [M+H]⁺

### Example 53

### 4-[(1-ethanimidoylpiperidin-4-yl)oxy]phenyl [5-[amino(imino)methyl]-2-oxo-1,3-benzoxazol-3(2H)-yl]acetate 2 trifluoroacetate

### Step 1 Synthesis of tert-butyl (5-cyano-2-oxo-1,3-benzoxazol-3(2H)-yl)acetate

3-Amino-4-hydroxybenzonitrile (711 mg, 5.30 mmol) and carbonyldiimidazole (860 mg, 5.30 mmol) were dissolved in DMF (18 mL), and the mixture was stirred at room temperature for 4 hr 30 min. To the reaction solution were added potassium carbonate (1.10 g, 7.95 mmol) and tert-butyl bromoacetate (0.933 mL, 6.36 mmol), and the mixture was stirred at room temperature for 2 hr, and at 45° C. for 1 hr. Potassium carbonate (484 mg, 5.30 mmol) and tert-butyl bromoacetate (0.777 mL, 5.30 mmol) were further added and the mixture was stirred at room temperature overnight. The solvent was evaporated under reduced pressure. The obtained residue was diluted with ethyl acetate, the organic phase was washed with water, 1N hydrochloric acid and saturated brine, and dried over anhydrous magnesium sulfate. The solvent was evaporated under reduced pressure and the obtained residue was purified by silica gel chromatography (hexane:ethyl acetate=1:1) to give the title compound.

yield: 867 mg (3.16 mmol) yield: 60%

¹H-NMR (CDCl₃, 400 MHz) δ1.50 (s, 9H), 4.48 (s, 2H), 7.16 (d, 1H, J=1.6 Hz), 7.32 (d, 1H, J=8.3 Hz), 7.50 (dd, 1H, J=8.3, 1.6 Hz).

### Step 2 Synthesis of ethyl [5-[amino(imino)methyl]-2-oxo-1,3-benzoxazol-3(2H)-yl]acetate trifluoroacetate

The operation similar to that in Example 4, step 2, was performed using the compound obtained in step 1 instead of N-(3-cyanophenyl)-methyl-L-alanine to give the title compound.

yield: 468 mg (1.24 mmol) yield: 57%

MS (ESI, m/z) 264 [M+H]⁺

¹H-NMR (DMSO-d₆, 400 MHz) δ1.24 (t, 2H, J=7.1 Hz), 4.20 (q, 2H, J=7.1 Hz), 4.81 (s, 2H), 7.70-7.60 (m, 2H), 7.83 (d, 1H, J=1.2 Hz), 9.14 (br s, 2H), 9.30 (br s, 2H).

### Step 3 Synthesis of [5-[amino(imino)methyl]-2-oxo-1,3-benzoxazol-3(2H)-yl]acetic acid hydrochloride

To the compound obtained in step 2 (465 mg, 1.23 mmol) were added water (2.0 mL) and 4N hydrochloric acid/1,4-dioxane solution (8.0 mL), and the mixture was stirred at 80° C. for 1 hr. The mixture was cooled to room temperature and the solvent was evaporated under reduced pressure. The obtained residue was diluted with water and lyophilized to give the title compound without purification.

yield: 342 mg (1.26 mmol) yield: quantitative

MS (ESI, m/z) 236 [M+H]⁺

¹H-NMR (DMSO-d₆, 400 MHz) δ4.69 (s, 2H), 7.75-7.55 (m, 2H), 7.90 (d, 1H, J=0.7 Hz), 9.17 (s, 2H), 9.37 (s, 2H), 13.51 (br s, 1H)

### Step 4 Synthesis of 4-[(1-ethanimidoylpiperidin-4-yl)oxy]phenyl [5-[amino(imino)methyl]-2-oxo-1,3-benzoxazol-3(2H)-yl]acetate 2 trifluoroacetate

The operation similar to that in Example 6, step 4, was performed using the compound obtained in step 3 instead of N-{3-[amino(imino)methyl]phenyl}-N-methyl-L-alanine hydrochloride to give the title compound.

yield: 20.3 mg (0.0299 mmol) yield: 10%

MS (ESI, m/z) 452 [M+H]⁺

¹H-NMR (DMSO-d₆, 400 MHz) δ1.83-1.67 (m, 2H), 2.11-1.99 (m, 2H), 2.28 (s, 3H), 3.59-3.43 (m, 2H), 3.81-3.68 (m, 2H), 4.73-4.64 (m, 1H), 5.11 (s, 2H), 7.09-7.03 (m, 2H), 7.19-7.12 (m, 2H), 7.71-7.63 (m, 2H), 7.94 (d, 1H, J=1.1 Hz), 8.59 (br s, 1H), 9.13 (br s, 1H), 9.21 (br s, 2H), 9.34 (br s, 2H).

### Example 54

### 4-[imino(pyrrolidin-1-yl)methyl]phenyl {6-[amino(imino)methyl]-1H-indol-1-yl}acetate 2 trifluoroacetate

### Step 1 Synthesis of tert-butyl (6-cyano-1H-indol-1-yl)acetate

To a solution (30 ml) of 6-cyanoindole (853 mg, 6.00 mmol) and cesium carbonate (2.00 g, 6.00 mmol) in DMF was added tert-butyl bromoacetate (0.880 mL, 6.00 mmol) and the mixture was stirred at room temperature for 5 hr. Cesium carbonate (400 mg, 1.20 mmol) and tert-butyl bromoacetate (0.176 mL, 1.20 mmol) were further added and the mixture was stirred at room temperature for 2 hr. The solvent was evaporated under reduced pressure. The obtained residue was diluted with ethyl acetate, washed with water, 0.5N hydrochloric acid, saturated aqueous sodium hydrogen carbonate solution and saturated brine, and the organic phase was dried over anhydrous magnesium sulfate. The solvent was evaporated under reduced pressure to give the title compound without purification.

yield: 1.62 g (6.32 mmol) yield: quantitative

¹H-NMR (CDCl₃, 400 MHz) δ1.46 (s, 9H), 4.76 (s, 2H), 6.62 (dd, 1H, J=3.2, 0.8 Hz), 7.28 (d, 1H, J=3.2 Hz), 7.34 (dd, 1H, J=8.2, 1.3 Hz), 7.59-7.56 (m, 1H), 7.67 (d, 1H, J=8.2 Hz).

### Step 2 Synthesis of ethyl {6-[amino(imino)methyl]-1H-indol-1-yl}acetate trifluoroacetate

The operation similar to that in Example 4, step 2, was performed using the compound obtained in step 1 instead of N-(3-cyanophenyl)-methyl-L-alanine to give the title compound.

yield: 1.36 g (3.79 mmol) yield: 59%

MS (ESI, m/z) 246 [M+H]⁺

### Step 3 Synthesis of {6-[amino(imino)methyl]-1H-indol-1-yl}acetic acid hydrochloride

The operation similar to that in Example 53, step 3, was performed using the compound obtained in step 2 instead of ethyl [5-[amino(imino)methyl]-2-oxo-1,3-benzoxazol-3(2H)-yl]acetate trifluoroacetate to give the title compound.

yield: 442 mg (1.74 mmol) yield: quantitative

MS (ESI, m/z) 218 [M+H]⁺

### Step 4 Synthesis of 4-[imino(pyrrolidin-1-yl)methyl]phenyl {6-[amino(imino)methyl]-1H-indol-1-yl}acetate 2 trifluoroacetate

The operation similar to that in Example 4, step 3, was performed using the compound obtained in step 3 instead of N-{3-[amino(imino)methyl]phenyl}-N-methyl-L-alanine hydrochloride to give the title compound.

yield: 5.59 mg (0.00905 mmol) yield: 3%

MS (ESI, m/z) 390 [M+H]⁺

### Example 55

### 4-[(1-ethanimidoylpiperidin-4-yl)oxy]phenyl {6-[amino(imino)methyl]-1H-indol-1-yl}acetate 2 trifluoroacetate

The operation similar to that in Example 6, step 4, was performed using {6-[amino(imino)methyl]-1H-indol-1-yl}acetic acid hydrochloride instead of N-{3-[amino(imino)methyl]phenyl}-N-methyl-L-alanine hydrochloride to give the title compound.

yield: 29.3 mg (0.0443 mmol) yield: 15%

MS (ESI, m/z) 434 [M+H]⁺

¹H-NMR (DMSO-d₆, 400 MHz) δ1.80-1.66 (2H, m), 2.11-1.99 (2H, m), 2.28 (3H, s), 3.57-3.47 (2H, m), 3.81-3.69 (2H, m), 4.71-4.63 (1H, m), 5.50 (2H, s), 6.68 (1H, d, J=3.2 Hz), 7.09-7.02 (2H, m), 7.18-7.10 (2H, m), 7.56-7.51 (1H, m), 7.82-7.73 (2H, m), 8.23 (1H, s), 8.61 (1H, br s), 9.05 (2H, br s), 9.23-9.12 (3H, m).

### Example 56

### 4-[imino(pyrrolidin-1-yl)methyl]phenyl 1-{3-[amino(imino)methyl]phenyl}-3-(trifluoromethyl)-1H-pyrazole-5-carboxylate 2 trifluoroacetate

### Step 1 Synthesis of 3-[5-(2-furyl)-3-(trifluoromethyl)-1H-pyrazol-1-yl]benzonitrile

To 3-aminobenzonitrile (1.18 g, 10.0 mmol) was added an aqueous solution (3 mL) of concentrated hydrochloric acid (10 mL) and sodium nitrite (690 mg, 10.0 mmol) at 0° C. and the mixture was stirred at said temperature for 30 min. To the reaction mixture was added a solution (4 mL) of tin chloride 2 hydrate (6.77 g, 30.0 mmol) in concentrated hydrochloric acid and the mixture was further stirred at 0° C. overnight. The precipitated solid was collected by filtration, washed with saturated brine and a mixed solvent of petroleum ether and diethyl ether, and dried. To the thus-obtained solid (2.56 g) were added acetic acid (30 mL) and 4,4,4-trifluoro-1-(2-furyl)-1,3-butanedione (1.17 mL, 7.94 mmol), and the mixture was stirred at 85° C. overnight, and further at 90° C. for 4 hr. The mixture was concentrated under reduced pressure and the obtained residue was diluted with ethyl acetate, and washed with water, 1N hydrochloric acid and saturated brine. The organic phase was dried over anhydrous magnesium sulfate. The solvent was evaporated under reduced pressure and the obtained residue was purified by silica gel chromatography (hexane:ethyl acetate=3:2) to give the title compound.

yield: 1.52 g (5.01 mmol) yield: 50%

MS (ESI, m/z) 304 [M+H]⁺

¹H-NMR (CDCl₃, 400 MHz) δ6.27 (dd, 1H, J=3.5, 0.7 Hz), 6.44 (dd, 1H, J=3.5, 1.8 Hz), 6.90 (s, 1H), 7.44 (dd, 1H, J=1.8, 0.7 Hz), 7.60 (t, 1H, J=8.1 Hz), 7.72-7.68 (m, 1H), 7.77-7.74 (m, 2H).

### Step 2 Synthesis of 1-(3-cyanophenyl)-3-(trifluoromethyl)-1H-pyrazole-5-carboxylic acid

To the compound obtained in step 1 (1.52 g, 5.01 mmol) were added acetonitrile (10 mL), carbon tetrachloride (10 mL) and ruthenium (III) chloride (160 mg, 0.771 mmol). To the mixture was added an aqueous solution (15 mL) of sodium periodate (4.82 g, 22.5 mmol), and the mixture was stirred at room temperature overnight. The mixture was filtered through celite and the obtained filtrate was diluted with ethyl acetate, washed with saturated aqueous sodium sulfite solution, and 5% aqueous sodium thiosulfate solution, and extracted with 2N aqueous sodium hydroxide solution. The aqueous phase was acidified with concentrated hydrochloric acid under ice-cooling, and extracted with dichloromethane. The organic phase was dried over anhydrous sodium sulfate to give the title compound without purification.

yield: 244 mg (0.868 mmol) yield: 17%

MS (ESI, m/z) 282 [M+H]⁺

### Step 3 Synthesis of 1-{3-[amino(imino)methyl]phenyl}-3-(trifluoromethyl)-1H-pyrazole-5-carboxylic acid hydrochloride

The operation similar to that in Example 4, step 2, was performed using the compound obtained in step 2 instead of N-(3-cyanophenyl)-methyl-L-alanine to give the title compound.

yield: 224 mg (0.669 mmol) yield: 69%

MS (ESI, m/z) 299 [M+H]⁺

### Step 4 Synthesis of 4-[imino(pyrrolidin-1-yl)methyl]phenyl 1-{(3-[amino(imino)methyl]phenyl}-3-(trifluoromethyl)-1H-pyrazole-5-carboxylate 2 trifluoroacetate

The operation similar to that in Example 4, step 3, was performed using the compound obtained in step 3 instead of N-{3-[amino(imino)methyl]phenyl}-N-methyl-L-alanine hydrochloride to give the title compound.

yield: 10.9 mg (0.0156 mmol) yield: 4.4%

MS (ESI, m/z) 471 [M+H]⁺

¹H-NMR (DMSO-d₆, 400 MHz) δ1.86 (quint, 2H), 2.05 (quint, 2H), 3.40-3.34 (m, 2H), 3.56-3.51 (m, 2H), 7.52-7.48 (m, 2H), 7.76-7.71 (m, 2H), 7.80 (t, 1H, J=8.0 Hz), 8.01-7.97 (m, 1H), 8.04 (d, 1H, J=0.5 Hz), 8.17-8.10 (m, 2H), 8.81 (br s, 1H), 9.31-9.25 (m, 3H), 9.44 (br s, 2H).

### Example 57

### 4-[(1-ethanimidoylpiperidin-4-yl)oxy]phenyl 1-{3-[amino(imino)methyl]phenyl}-3-(trifluoromethyl)-1H-pyrazole-5-carboxylate 2 trifluoroacetate

The operation similar to that in Example 6, step 4, was performed using 1-{3-[amino(imino)methyl]phenyl}-3-(trifluoromethyl)-1H-pyrazole-5-carboxylic acid hydrochloride instead of N-{3-[amino(imino)methyl]phenyl}-N-methyl-L-alanine hydrochloride to give the title compound.

yield: 21.4 mg (0.0288 mmol) yield: 9.6%

MS (ESI, m/z) 515 [M+H]⁺

¹H-NMR (DMSO-d₆, 400 MHz) δ1.82-1.67 (m, 2H), 2.11-1.99 (m, 2H), 2.28 (s, 3H), 3.57-3.47 (m, 2H), 3.81-3.69 (m, 2H), 4.72-4.64 (m, 1H), 7.09-7.01 (m, 2H), 7.20-7.14 (m, 2H), 7.80 (t, 1H, J=8.0 Hz), 8.02-7.94 (m, 2H), 8.12-8.06 (m, 1H), 8.17-8.12 (m, 1H), 8.62 (br s, 1H), 9.16 (br s, 1H), 9.50-9.37 (m, 4H).

The structural formulas of the compounds described in Examples are shown in Table 1-Table 3. TFA in the formulas is trifluoroacetic acid.

TABLE 1

| | |
|---|---|
| compound of. Ex. 1 | 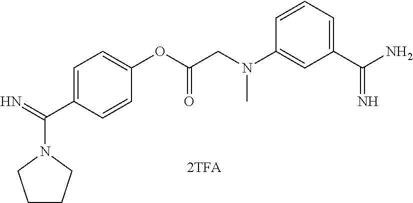 [SMILES: CN(CC(=O)Oc1ccc(C(=N)N2CCCC2)cc1)c1cccc(C(=N)N)c1] |
| compound of. Ex. 2 | 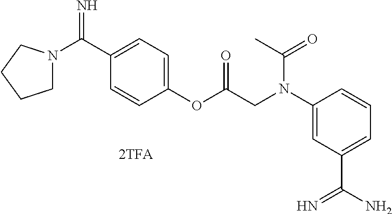 [SMILES: CC(=O)N(CC(=O)Oc1ccc(C(=N)N2CCCC2)cc1)c1cccc(C(=N)N)c1] |
| compound of. Ex. 3 | 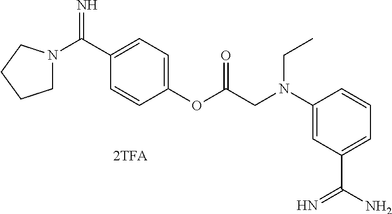 [SMILES: CCN(CC(=O)Oc1ccc(C(=N)N2CCCC2)cc1)c1cccc(C(=N)N)c1] |
| compound of. Ex. 4 | 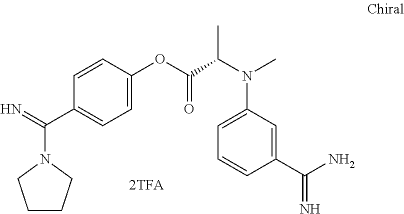 [SMILES: C[C@@H](C(=O)Oc1ccc(C(=N)N2CCCC2)cc1)N(C)c1cccc(C(=N)N)c1] |
| compound of. Ex. 5 | 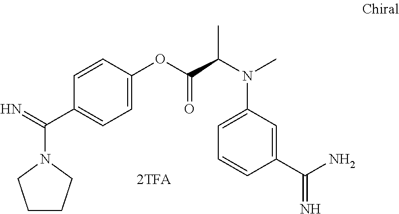 [SMILES: C[C@H](C(=O)Oc1ccc(C(=N)N2CCCC2)cc1)N(C)c1cccc(C(=N)N)c1] |
| compound of. Ex. 6 | 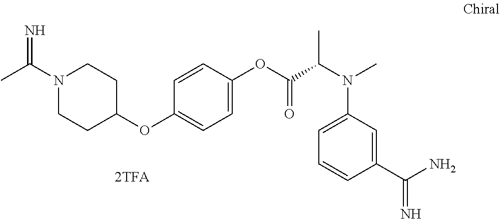 [SMILES: CC(=N)N1CCC(Oc2ccc(OC(=O)[C@H](C)N(C)c3cccc(C(=N)N)c3)cc2)CC1] |
| compound of. Ex. 7 | 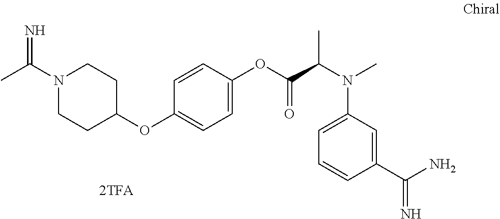 [SMILES: CC(=N)N1CCC(Oc2ccc(OC(=O)[C@@H](C)N(C)c3cccc(C(=N)N)c3)cc2)CC1] |
| compound of. Ex. 8 | 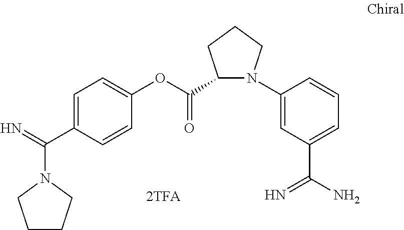 [SMILES: N=C(N)c1cccc(N2CCC[C@H]2C(=O)Oc2ccc(C(=N)N3CCCC3)cc2)c1] |
| compound of. Ex. 9 | 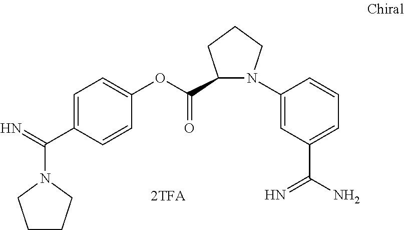 [SMILES: N=C(N)c1cccc(N2CCC[C@@H]2C(=O)Oc2ccc(C(=N)N3CCCC3)cc2)c1] |
| compound of. Ex. 10 | 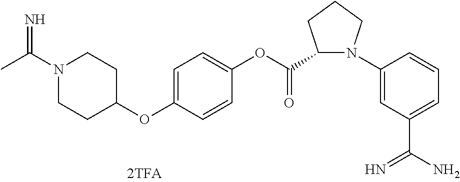 [SMILES: CC(=N)N1CCC(Oc2ccc(OC(=O)[C@@H]3CCCN3c3cccc(C(=N)N)c3)cc2)CC1] |
| compound of. Ex. 11 | 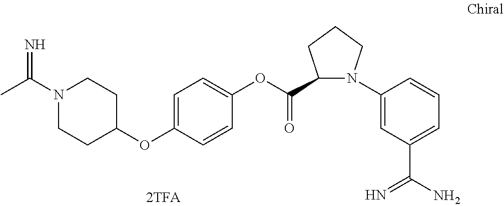 [SMILES: CC(=N)N1CCC(Oc2ccc(OC(=O)[C@H]3CCCN3c3cccc(C(=N)N)c3)cc2)CC1] |
| compound of. Ex. 12 | 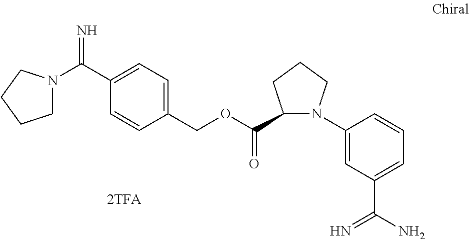 [SMILES: N=C(N)c1cccc(N2CCC[C@@H]2C(=O)OCc2ccc(C(=N)N3CCCC3)cc2)c1] |
| compound of. Ex. 13 | 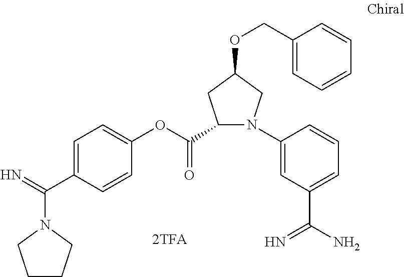 [SMILES: N=C(N)c1cccc(N2C[C@H](OCc3ccccc3)C[C@H]2C(=O)Oc2ccc(C(=N)N3CCCC3)cc2)c1] |
| compound of. Ex. 14 | 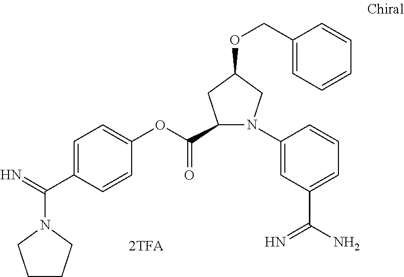 [SMILES: N=C(N)c1cccc(N2C[C@H](OCc3ccccc3)C[C@@H]2C(=O)Oc2ccc(C(=N)N3CCCC3)cc2)c1] |
| compound of. Ex. 15 | 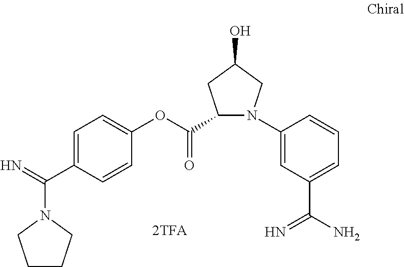 [SMILES: N=C(N)c1cccc(N2C[C@H](O)C[C@H]2C(=O)Oc2ccc(C(=N)N3CCCC3)cc2)c1] |
| compound of. Ex. 16 | 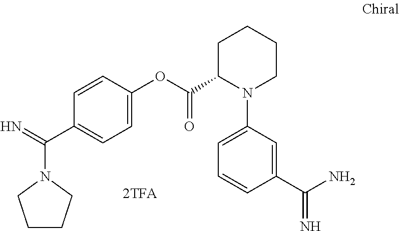 [SMILES: N=C(N)c1cccc(N2CCCC[C@H]2C(=O)Oc2ccc(C(=N)N3CCCC3)cc2)c1] |
| compound of. Ex. 17 | 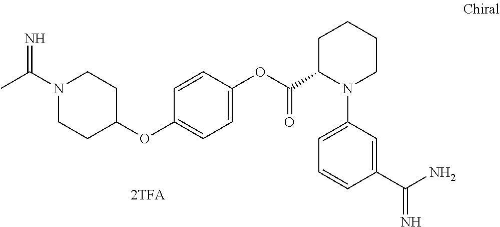 [SMILES: CC(=N)N1CCC(Oc2ccc(OC(=O)[C@@H]3CCCCN3c3cccc(C(=N)N)c3)cc2)CC1] |
| compound of. Ex. 18 | 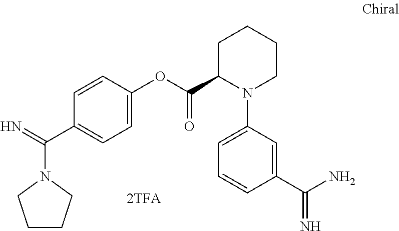 [SMILES: N=C(N)c1cccc(N2CCCC[C@@H]2C(=O)Oc2ccc(C(=N)N3CCCC3)cc2)c1] |
| compound of. Ex. 19 | 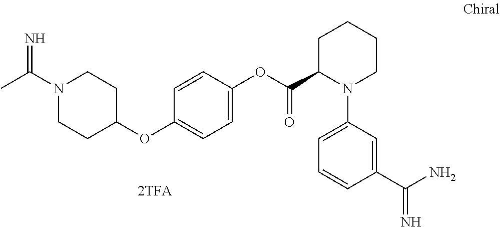 [SMILES: CC(=N)N1CCC(Oc2ccc(OC(=O)[C@H]3CCCCN3c3cccc(C(=N)N)c3)cc2)CC1] |
| compound of. Ex. 20 | 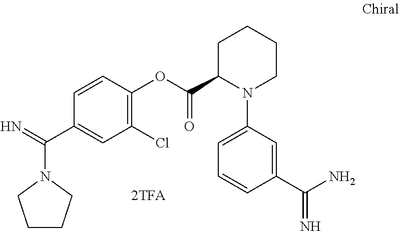 [SMILES: N=C(N)c1cccc(N2CCCC[C@@H]2C(=O)Oc2ccc(C(=N)N3CCCC3)cc2Cl)c1] |

TABLE 2

| | |
|---|---|
| compound of. Ex. 21 | 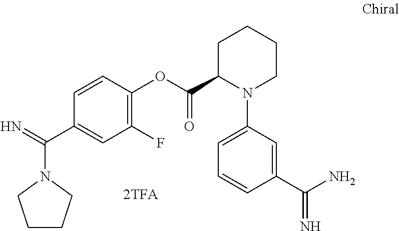 [SMILES: N=C(N)c1cccc(N2CCCC[C@@H]2C(=O)Oc2ccc(C(=N)N3CCCC3)cc2F)c1] |
| compound of. Ex. 22 | 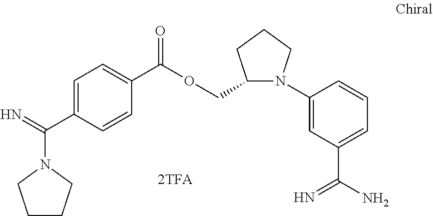 [SMILES: N=C(N)c1cccc(N2CCC[C@H]2COC(=O)c2ccc(C(=N)N3CCCC3)cc2)c1] |
| compound of. Ex. 23 | 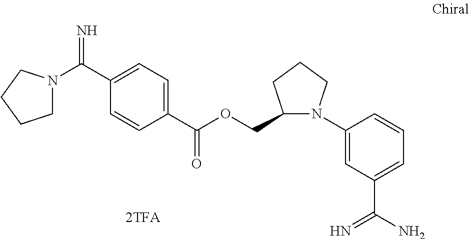 [SMILES: N=C(N)c1cccc(N2CCC[C@@H]2COC(=O)c2ccc(C(=N)N3CCCC3)cc2)c1] |
| compound of. Ex. 24 | 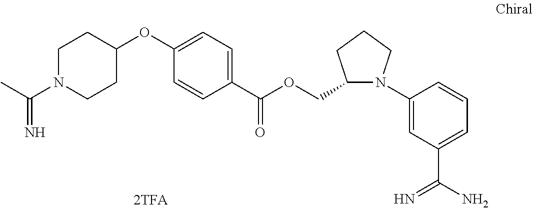 [SMILES: CC(=N)N1CCC(Oc2ccc(C(=O)OC[C@@H]3CCCN3c3cccc(C(=N)N)c3)cc2)CC1] |
| compound of. Ex. 25 | 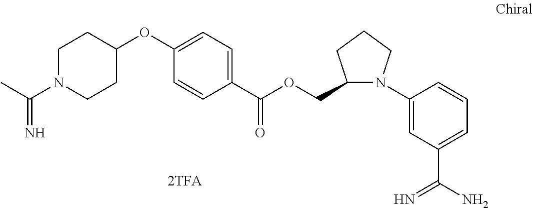 [SMILES: CC(=N)N1CCC(Oc2ccc(C(=O)OC[C@H]3CCCN3c3cccc(C(=N)N)c3)cc2)CC1] |
| compound of. Ex. 26 | 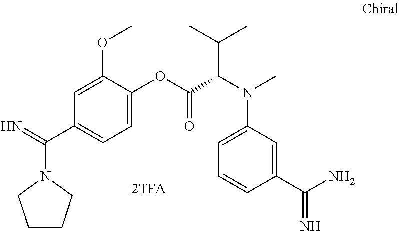 [SMILES: COc1cc(C(=N)N2CCCC2)ccc1OC(=O)[C@H](C(C)C)N(C)c1cccc(C(=N)N)c1] |
| compound of. Ex. 27 | 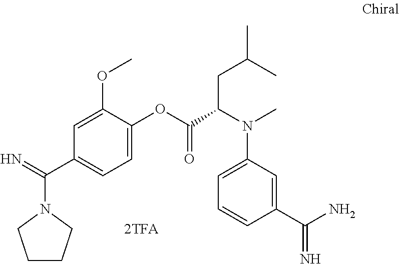 [SMILES: COc1cc(C(=N)N2CCCC2)ccc1OC(=O)[C@H](CC(C)C)N(C)c1cccc(C(=N)N)c1] |
| compound of. Ex. 28 | 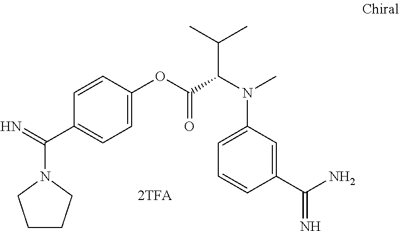 [SMILES: CC(C)[C@@H](C(=O)Oc1ccc(C(=N)N2CCCC2)cc1)N(C)c1cccc(C(=N)N)c1] |
| compound of. Ex. 29 | 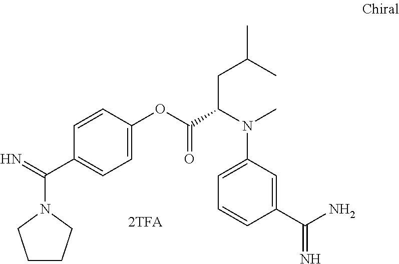 [SMILES: CC(C)C[C@@H](C(=O)Oc1ccc(C(=N)N2CCCC2)cc1)N(C)c1cccc(C(=N)N)c1] |
| compound of. Ex. 30 | 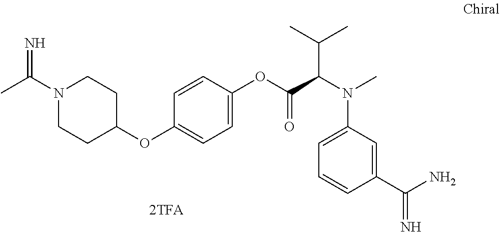 [SMILES: CC(=N)N1CCC(Oc2ccc(OC(=O)[C@@H](C(C)C)N(C)c3cccc(C(=N)N)c3)cc2)CC1] |
| compound of. Ex. 31 | 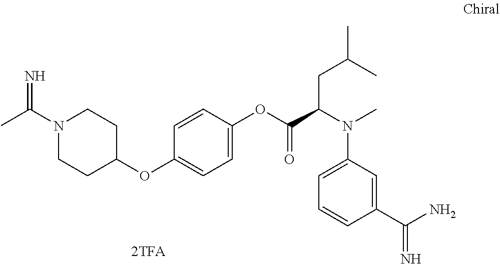 [SMILES: CC(=N)N1CCC(Oc2ccc(OC(=O)[C@@H](CC(C)C)N(C)c3cccc(C(=N)N)c3)cc2)CC1] |
| compound of. Ex. 32 | 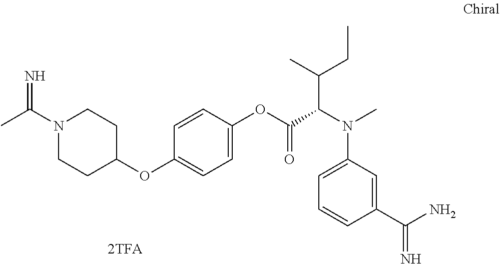 [SMILES: CCC(C)[C@@H](C(=O)Oc1ccc(OC2CCN(C(C)=N)CC2)cc1)N(C)c1cccc(C(=N)N)c1] |
| compound of. Ex. 33 | 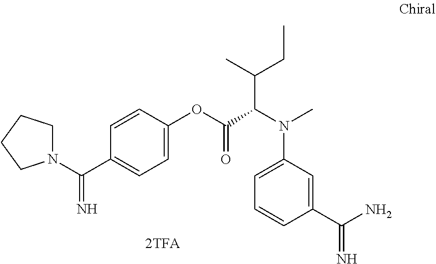 [SMILES: CCC(C)[C@@H](C(=O)Oc1ccc(C(=N)N2CCCC2)cc1)N(C)c1cccc(C(=N)N)c1] |
| compound of. Ex. 34 | 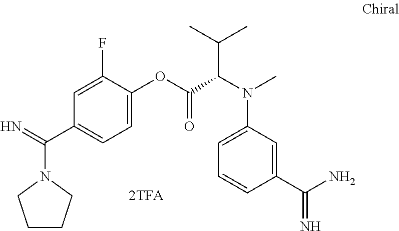 [SMILES: CC(C)[C@@H](C(=O)Oc1ccc(C(=N)N2CCCC2)cc1F)N(C)c1cccc(C(=N)N)c1] |
| compound of. Ex. 35 | 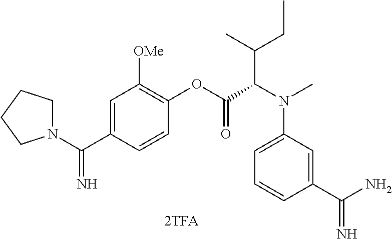 [SMILES: CCC(C)[C@@H](C(=O)Oc1ccc(C(=N)N2CCCC2)cc1OC)N(C)c1cccc(C(=N)N)c1] |
| compound of. Ex. 36 | 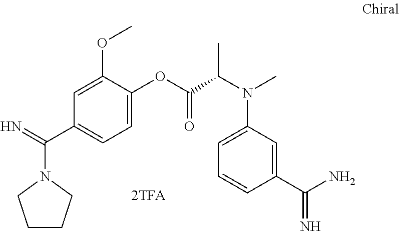 [SMILES: COc1cc(C(=N)N2CCCC2)ccc1OC(=O)[C@H](C)N(C)c1cccc(C(=N)N)c1] |
| compound of. Ex. 37 | 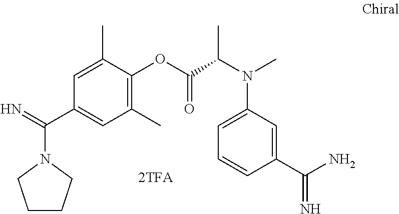 [SMILES: Cc1cc(C(=N)N2CCCC2)cc(C)c1OC(=O)[C@H](C)N(C)c1cccc(C(=N)N)c1] |
| compound of. Ex. 38 | 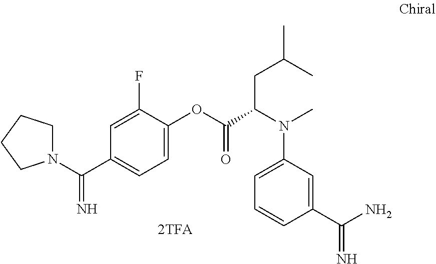 [SMILES: CC(C)C[C@@H](C(=O)Oc1ccc(C(=N)N2CCCC2)cc1F)N(C)c1cccc(C(=N)N)c1] |
| compound of. Ex. 39 | 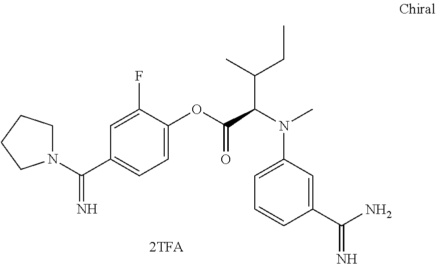 [SMILES: CCC(C)[C@H](C(=O)Oc1ccc(C(=N)N2CCCC2)cc1F)N(C)c1cccc(C(=N)N)c1] |
| compound of. Ex. 40 | 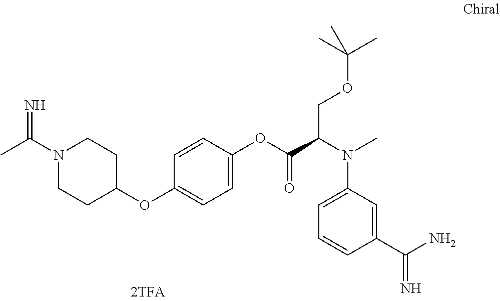 [SMILES: CC(=N)N1CCC(Oc2ccc(OC(=O)[C@@H](COC(C)(C)C)N(C)c3cccc(C(=N)N)c3)cc2)CC1] |

TABLE 3

| | |
|---|---|
| compound of. Ex. 41 | 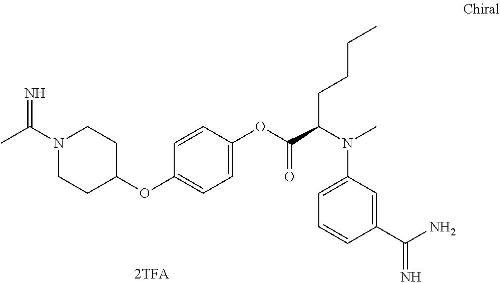 [SMILES: CCCC[C@H](C(=O)Oc1ccc(OC2CCN(C(C)=N)CC2)cc1)N(C)c1cccc(C(=N)N)c1] |
| compound of. Ex. 42 | 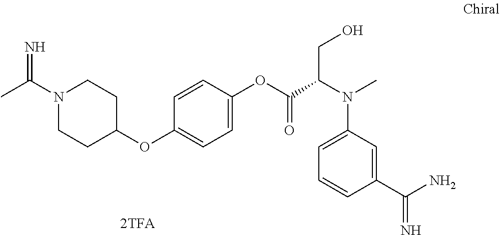 [SMILES: CC(=N)N1CCC(Oc2ccc(OC(=O)[C@H](CO)N(C)c3cccc(C(=N)N)c3)cc2)CC1] |
| compound of. Ex. 43 | 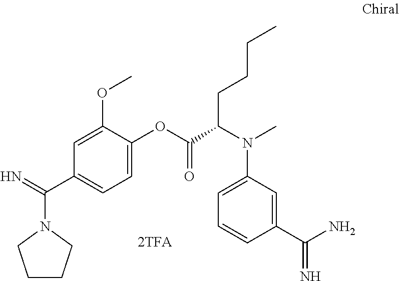 [SMILES: CCCC[C@@H](C(=O)Oc1ccc(C(=N)N2CCCC2)cc1OC)N(C)c1cccc(C(=N)N)c1] |
| compound of. Ex. 44 | 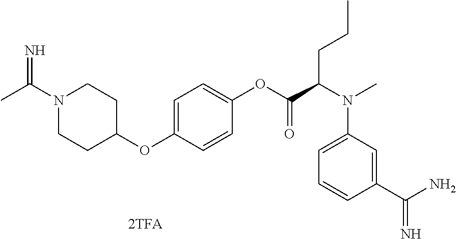 [SMILES: CCC[C@H](C(=O)Oc1ccc(OC2CCN(C(C)=N)CC2)cc1)N(C)c1cccc(C(=N)N)c1] |
| compound of. Ex. 45 | 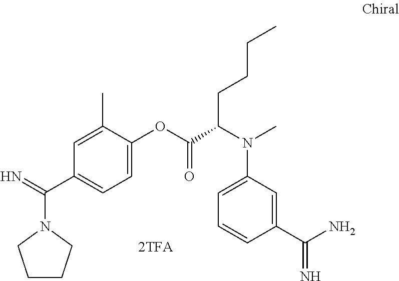 [SMILES: CCCC[C@@H](C(=O)Oc1ccc(C(=N)N2CCCC2)cc1C)N(C)c1cccc(C(=N)N)c1] |
| compound of. Ex. 46 | 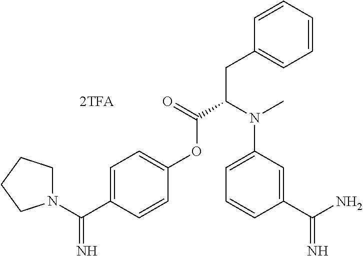 [SMILES: CN(c1cccc(C(=N)N)c1)[C@@H](Cc1ccccc1)C(=O)Oc1ccc(C(=N)N2CCCC2)cc1] |
| compound of. Ex. 47 | 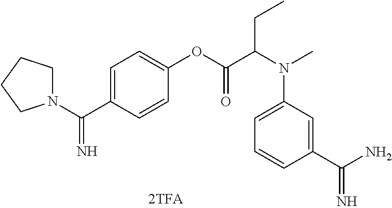 [SMILES: CCC(C(=O)Oc1ccc(C(=N)N2CCCC2)cc1)N(C)c1cccc(C(=N)N)c1] |
| compound of. Ex. 48 | 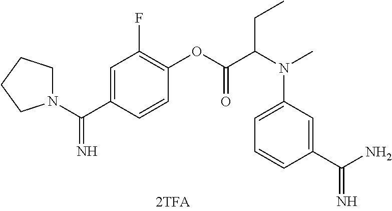 [SMILES: CCC(C(=O)Oc1ccc(C(=N)N2CCCC2)cc1F)N(C)c1cccc(C(=N)N)c1] |
| compound of. Ex. 49 | 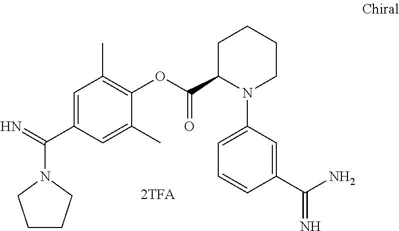 [SMILES: Cc1cc(C(=N)N2CCCC2)cc(C)c1OC(=O)[C@H]1CCCCN1c1cccc(C(=N)N)c1] |
| compound of. Ex. 50 | 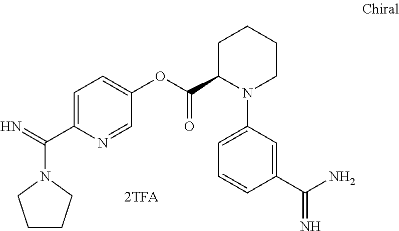 [SMILES: N=C(N)c1cccc(N2CCCC[C@@H]2C(=O)Oc2ccc(C(=N)N3CCCC3)nc2)c1] |
| compound of. Ex. 51 | 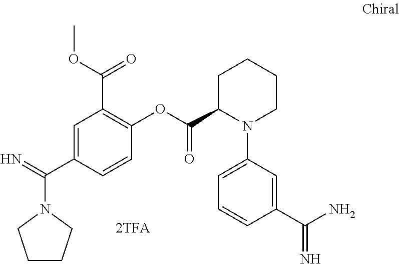 [SMILES: COC(=O)c1cc(C(=N)N2CCCC2)ccc1OC(=O)[C@H]1CCCCN1c1cccc(C(=N)N)c1] |
| compound of. Ex. 52 | 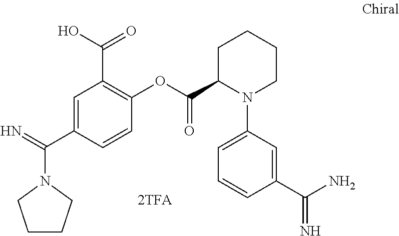 [SMILES: N=C(N)c1cccc(N2CCCC[C@@H]2C(=O)Oc2ccc(C(=N)N3CCCC3)cc2C(=O)O)c1] |
| compound of. Ex. 53 | 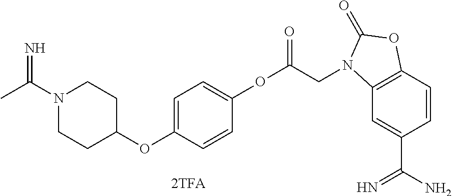 [SMILES: CC(=N)N1CCC(Oc2ccc(OC(=O)Cn3c(=O)oc4ccc(C(=N)N)cc43)cc2)CC1] |
| compound of. Ex. 54 | 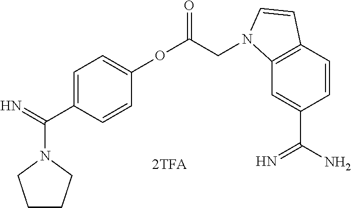 [SMILES: N=C(N)c1ccc2ccn(CC(=O)Oc3ccc(C(=N)N4CCCC4)cc3)c2c1] |
| compound of. Ex. 55 | 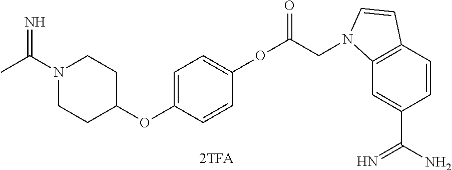 [SMILES: CC(=N)N1CCC(Oc2ccc(OC(=O)Cn3ccc4ccc(C(=N)N)cc43)cc2)CC1] |
| compound of. Ex. 56 | 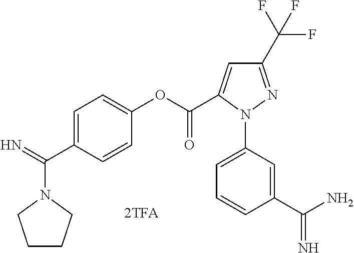 [SMILES: N=C(N)c1cccc(-n2nc(C(F)(F)F)cc2C(=O)Oc2ccc(C(=N)N3CCCC3)cc2)c1] |
| compound of. Ex. 57 | 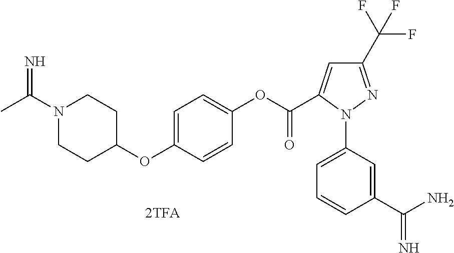 [SMILES: CC(=N)N1CCC(Oc2ccc(OC(=O)c3cc(C(F)(F)F)nn3-c3cccc(C(=N)N)c3)cc2)CC1] |

### Experimental Example 1 Measurement of Inhibitory Activity Against Activated Factor X Activity

Using a 96-well plate (#3396, Costar), 100 mM Tris-HC1 buffer (130 μL) containing 0.02% Tween 20, 0.1% PEG6000 and 0.2M NaCl was admixed with 0.015 U/ml FXa (10 μL) and a test compound (10 μL) for 10 min and a color developing substrate 0.2 mM S-2222 (50 μL) was added. Using a microplate reader Benchmark Plus (BIO-RAD), the reaction rate was measured from the time course changes at absorbance 405 nm. Using the reaction rate of the control as 100%, the negative logarithmic value of the concentration that can suppress the reaction rate of the control by 50% was taken as the pIC₅₀ value. The results are shown in Table 4.

### Experimental Example 2 Measurement of Inhibitory Activity Against Activated Factor II (FIIa, Thrombin)

Using a 96-well plate (#3396, Costar), 100 mM Tris-HCl buffer (130 μL) containing 0.02% Tween 20, 0.1% PEG6000 and 0.2M NaCl was admixed with 0.125 U/ml activated factor IIa (thrombin) (10 μL) and a test compound (10 μL) for 10 min and a color developing substrate 0.1 mM S-2238 (50 μL) was added. Using a microplate reader Benchmark Plus (BIO-RAD), the reaction rate was measured from the time course changes at absorbance 405 nm. Using the reaction rate of the control as 100%, the negative logarithmic value of the concentration that can suppress the reaction rate of the control by 50% was taken as the pIC₅₀ value. The results are shown in Table 4.

### Experimental Example 3-1 Measurement of Anti-Blood Coagulant Activity

Examples 1, 8, 9, 11, 13, 16-19 and 22 were measured according to the following method.

An aPTT measurement method using totally automatic blood coagulation time measuring apparatus Sysmex CA-1500 was used. A 10 mg/ml DDVP solution (DDVP standard product, Wako) (4 μL) and a test compound solution (20 μL) were placed in a sample tube (MS-18, Japan Medical Science), human plasma (standard human plasma for blood coagulation test, GCH-100A, Sysmex) (180 μL) was added and the mixture was used as a test sample. The test sample (50 μL) was incubated at 37° C. for 1 min, data fi APTT (rabbit brain-derived cephalin, DADE Behiring) (50 μL) was added, and the mixture was further incubated at 37° C. for 2 min. To the sample solution was added 0.02M calcium chloride (50 μL), and the time until plasma coagulation was automatically measured.

As the anti-blood coagulant activity, the negative logarithmic value of the concentration that prolongs aPTT of the control 2 times is shown as paPTT2. The results are shown in Table 4.

### Experimental Example 3-2 Measurement of Anti-Blood Coagulant Activity

Example 4-7, 15, 20, 21, 28, 29, 33, 36, 39-47, 50-52 and 57 were measured according to the following method.

An aPTT measurement method using totally automatic blood coagulation time measuring apparatus Sysmex Cs-2000i was used. A 10 mg/ml DDVP solution (DDVP standard product, Wako) (8 μL) and a test compound solution (40 μL) were placed in a sample tube (MS-18, Japan Medical Science), human plasma (standard human plasma for blood coagulation test, GCH-100A, Sysmex) (360 μL) was added and the mixture was used as a test sample. The test sample (50 μL) was incubated at 37° C. for 1 min, data fi APTT (rabbit brain-derived cephalin, DADE Behiring) (50 μL) was added, and the mixture was further incubated at 37° C. for 2 min. To the sample solution was added 0.02M calcium chloride (50 μL), and the time until plasma coagulation was automatically measured.

As the anti-blood coagulant activity, the negative logarithmic value of the concentration that prolongs aPTT of the control 2 times is shown as paPTT2. The results are shown in Table 4 and Table 5.

TABLE 4

| | FXa (pIC₅₀) | FIIa (pIC₅₀) | paPTT2 |
|---|---|---|---|
| compound of Ex. 1 | 7.34 | <4.0 | 5.48 |
| compound of Ex. 4 | 7.87 | 4.08 | 6.89 |
| compound of Ex. 5 | 7.70 | 4.42 | 6.26 |
| compound of Ex. 6 | 7.84 | 4.48 | 6.81 |
| compound of Ex. 7 | 7.67 | 4.48 | 6.85 |
| compound of Ex. 8 | 6.55 | 4.59 | 5.66 |
| compound of Ex. 9 | 7.70 | 6.16 | 6.69 |
| compound of Ex. 11 | 6.98 | 6.46 | 6.25 |
| compound of Ex. 13 | 6.47 | 6.54 | 5.59 |
| compound of Ex. 15 | 6.65 | 4.55 | 5.68 |
| compound of Ex. 16 | 7.82 | 4.15 | 6.85 |
| compound of Ex. 17 | 7.39 | 4.27 | 6.33 |
| compound of Ex. 18 | 8.34 | 5.53 | 6.64 |
| compound of Ex. 19 | 8.11 | 5.47 | 6.89 |
| compound of Ex. 20 | 8.19 | 5.92 | 6.93 |
| compound of Ex. 21 | 8.29 | 5.72 | 6.94 |
| compound of Ex. 22 | 6.40 | <4 | 6.03 |

TABLE 5

| | FXa (pIC₅₀) | FIIa (pIC₅₀) | paPTT2 |
|---|---|---|---|
| compound of Ex. 28 | 7.51 | 4.85 | 6.55 |
| compound of Ex. 29 | 7.95 | 4.59 | 6.72 |
| compound of Ex. 33 | 8.09 | 4.88 | 6.80 |
| compound of Ex. 36 | 7.50 | 5.15 | 6.67 |
| compound of Ex. 39 | 7.82 | 4.59 | 6.50 |
| compound of Ex. 40 | 8.35 | 4.46 | 6.53 |
| compound of Ex. 41 | 8.53 | 5.66 | 6.53 |
| compound of Ex. 42 | 7.86 | 4.29 | 6.64 |
| compound of Ex. 43 | 8.51 | 6.01 | 6.90 |
| compound of Ex. 44 | 8.50 | 5.76 | 6.87 |
| compound of Ex. 45 | 8.52 | 5.59 | 6.71 |
| compound of Ex. 46 | 8.53 | 4.81 | 6.87 |
| compound of Ex. 47 | 8.09 | 4.65 | 6.82 |
| compound of Ex. 50 | 8.26 | 5.44 | 6.66 |
| compound of Ex. 51 | 7.85 | 5.65 | 6.79 |
| compound of Ex. 52 | 7.74 | <4 | 6.35 |
| compound of Ex. 57 | 8.27 | 5.16 | |

Experimental Example 4 Evaluation of stability in plasma

To human plasma (495 μL) was added 5 μL from a solution of the test compound prepared to 200 μM (final drug solution concentration 2 μM), and the mixture was incubated at 37° C. At 0 min, 1 min, 2 min, 5 min and 10 min from the addition of the drug solution, 50 μL each was sampled, 0.1 mg/ml DDVP-containing acetonitrile (350 μL) was added, and the reaction was discontinued by blending them. After discontinuation of the reaction, a deproteination treatment was performed by a centrifugation operation at 15000 rpm for 5 min. The centrifugation supernatant (350 μL) was dried to solidness, concentrated, dissolved in aqueous solution (100 μL) of 20% acetonitrile and 0.1% formic acid and the mixture was subjected to the measurement by LC/MS/MS.

The half-life (T1/2) was calculated using initial 3rd-4th time points. The results are shown in Table 6.

### Experimental Example 5 Evaluation of Stability in Liver S9 Fraction

Metabolism reaction mixed solution (0.1 mM EDTA-100 mM potassium phosphate buffer (pH 7.4), 2 mg/mL human liver S9 fraction, 0.5 mM oxidized nicotinamide adenine dinucleotide phosphate, 5 mM glucose-6-phosphoric acid, 1 unit/mL glucose-6-phosphate dehydrogenase) was pre-warmed at 37° C. for 5 min. After pre-warming, DMSO solution of the substrate was added such that the concentration of the final drug solution was 2 μM, and the metabolism reaction was started at 37° C. A given amount was sampled at 0 min, 5 min, 10 min and 30 min from the start of the reaction, 0.1 mg/mL dichlorvos-containing acetonitrile was added, and blended to discontinue the reaction. After discontinuation of the reaction, a deproteination treatment was performed by a centrifugation operation at 15000 rpm for 5 min. To the centrifugation supernatant was added 0.3% aqueous formic acid solution, admixed and the mixture was subjected to the measurement by LC/MS/MS. Then, as the residual ratio (%) at 30 min later, the ratio relative to the concentration at 0 min was calculated. The results are shown in Table 6.

TABLE 6

| | T½ | residual ratio (%) of liver |
|---|---|---|
| | (min) | S9 fraction 30 min later |
| compound of Ex. 4 | 4.0 | 57 |
| compound of Ex. 5 | 3.7 | 64 |
| compound of Ex. 11 | <1 | 85 |
| compound of Ex. 13 | 8.2 | 86 |
| compound of Ex. 22 | <1 | 80 |
| compound of Ex. 23 | 0.2 | 76 |
| compound of Ex. 24 | 0.5 | 92 |

### INDUSTRIAL APPLICABILITY

As shown in the aforementioned Experimental Examples, the compound represented by the formula (1-1), (1-2) or (1-3) and a pharmaceutically acceptable salt thereof has a high FXa inhibitory activity and anti-(blood) coagulation action, and can be used as an anti-(blood) coagulation drug (agent); for example, a therapeutic or prophylactic drug for reocclusion and restenosis after blood vessel reconstruction in various diseases wherein an FXa-dependent coagulation process is involved in the pathology, such as thrombus formation in extracorporeal blood circulation, cerebral infarction, cerebral thrombus, cerebral embolism, transient cerebral ischemic attack (TIA), acute and chronic myocardial infarction, unstable angina pectoris, pulmonary obliteration, peripheral arterial obstruction, deep vein thrombosis, disseminated intravascular coagulation syndrome, thrombus formation after artificial blood vessel operation and artificial valve replacement, reocclusion and restenosis after coronary-artery bypass surgery, percutaneous transluminal coronary angioplasty (PTCA) or percutaneous transluminal coronary recanalization (PTCR) and the like.

Particularly, a compound represented by the formula (1-1), (1-2) or (1-3) and a pharmaceutically acceptable salt thereof is useful as an anti-(blood) coagulation drug (agent) for an extracorporeal blood circulation circuit (e.g., hemodialyzer, artificial heart lung apparatus etc.).

In addition, a compound represented by the formula (1-1), (1-2) or (1-3) and a pharmaceutically acceptable salt thereof are rapidly cleared from the blood. That is, they have a short half-life in blood, and therefore, facilitate hemostasis when bleeding symptom is observed after administration and are useful as an anti-(blood) coagulation drug (agent) that can be used safely.

moreover, a compound represented by the formula (1-1), (1-2) or (1-3) and a pharmaceutically acceptable salt thereof show a low thrombin inhibitory activity, are FXa selective inhibitors, and are anti-(blood) coagulation drugs (agents) that can be used safely in view of the bleeding risk.

A low-molecular-weight FXa inhibitor, for example, a compound represented by the formula (1-1), (1-2) or (1-3) and a pharmaceutically acceptable salt thereof is useful as an anti-(blood) coagulation drug (agent) to be used during/for an extracorporeal blood circulation/extracorporeal blood circulation circuit.

Particularly, a selective low-molecular-weight FXa inhibitor which is rapidly cleared from the blood, or with a short half-life in blood; for example, a compound represented by the formula (1-1), (1-2) or (1-3) can be safely and conveniently used as an anti-(blood) coagulation drug (agent) for the prevention of blood coagulation in an extracorporeal blood circulation circuit, and is useful since a treatment of hemostasis and attention required after completion of the extracorporeal blood circulation can be clearly less.

Also, the present invention can also provide a method of preventing thrombus formation in an extracorporeal blood circulation circuit, which includes incorporating a low-molecular-weight FXa inhibitor as a constituent element of an extracorporeal blood circulation circuit.

This application is based on patent application No. 2010-073444 filed in Japan, the contents of which are encompassed in full herein.

1. An amidinoaniline compound represented by formula (1-1): 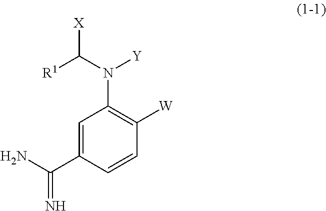
wherein:
X is a hydrogen atom, or a C₁₋₁₀ alkyl group optionally having substituent(s),
Y is a hydrogen atom, a C₁₋₁₀ alkyl group optionally having substituent(s), or an acyl group optionally having substituent(s),
W is a hydrogen atom, a hydroxyl group, an amino group, a C₁₋₁₀ alkyl group optionally having substituent(s), a C₁₋₁₀ alkoxy group optionally having substituent(s), a C₁₋₁₀ acyloxy group optionally having substituent(s), a carbamoyloxy group optionally having substituent(s), a C₁₋₁₀ alkylamino group optionally having substituent(s), a C₁₋₁₀ alkylthio group optionally having substituent(s), a C₁₋₁₀ acylamino group optionally having substituent(s), a carboxyl group, a carbamoyl group optionally having substituent(s), a thiocarbamoyl group optionally having substituent(s), a halogen atom, a cyano group, or a nitro group, or
X and Y are optionally bonded to each other to form a nitrogen-containing heterocycle optionally having substituent(s), or
Y and W are optionally bonded to each other to form a nitrogen-containing heterocycle optionally having substituent(s),
R¹ is a group represented by formula (2-1) or (2-2), provided that when R¹ is a group represented by formula (2-2), X is not a hydrogen atom, 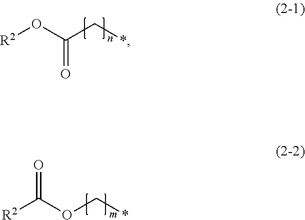
wherein:
n and m are each an integer of 0-2,
R² is a group represented by formula (3): 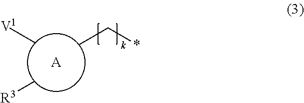
wherein:
k is an integer of 0-2, ring A is a C₆₋₁₀ aryl ring, a C₁₋₁₀ heteroaryl ring, a C₂₋₈ nitrogen-containing nonaromatic heterocycle, or a C₃₋₁₀ cycloalkyl ring,
V¹ is a hydrogen atom, a hydroxyl group, a halogen atom, an amino group, a C₁₋₁₀ alkyl group optionally having substituent(s), a C₁₋₁₀ alkoxy group optionally having substituent(s), a C₁₋₁₀ alkylamino group optionally having substituent(s), a C₁₋₁₀ alkylthio group optionally having substituent(s), a cyano group, a nitro group, a carboxyl group, a carbamoyl group optionally having substituent(s) or a C₂₋₁₀ alkoxycarbonyl group optionally having substituent(s),
R³ is a group represented by formula (4-1) or (4-2): 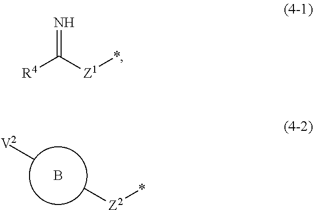
wherein:
Z¹ is —NH— or a single bond,
R⁴ is a C₁₋₆ alkyl group, an amino group optionally substituted by a C₁₋₁₀ alkyl group or a C₂₋₈ nitrogen-containing nonaromatic heterocyclic group bonded by a nitrogen, in formula (4-2),
ring B is a C₁₋₁₀ heteroaryl ring, or a C₂₋₈ nitrogen-containing nonaromatic heterocycle,
Z² is a single bond, —NH— optionally substituted by a C₁₋₆ alkyl group, an oxygen atom, a sulfur atom, a methylene group, or —CO—,
V² is a hydrogen atom, a halogen atom, an amidino group optionally substituted by a C₁₋₆ alkyl group, a guanidino group optionally substituted by a C₁₋₆ alkyl group, or a C₁₋₆ alkyl group optionally having an imino group at the 1-position,
or a pharmaceutically acceptable salt thereof.

2. An amidinoaniline compound according to claim 1, wherein X and Y are each a C₁₋₆ alkyl group optionally having substituent(s), or a pharmaceutically acceptable salt thereof.

3. An amidinoaniline compound according to claim 2, wherein:
ring A is a benzene ring, a pyridine ring, a thiophene ring, a piperidine ring, or a piperazine ring, and
V¹ is a hydrogen atom, a halogen atom, a C₁₋₆ alkyl group, or a C₁₋₆ alkoxy group,
or a pharmaceutically acceptable salt thereof.

4. An amidinoaniline derivative according to claim 3, wherein:
(1) R⁴ is an amino group, a C₁₋₁₀ alkylamino group, or a C₂₋₈ nitrogen-containing nonaromatic heterocyclic group bonded by a nitrogen; or
(2) ring B is a C₂₋₈ nitrogen-containing nonaromatic heterocycle,
Z² is an oxygen atom, a sulfur atom or a methylene group, and
V² is a hydrogen atom, a halogen atom, an amidino group, or a C₁₋₆ alkyl group optionally having an imino group at the 1-position,
or a pharmaceutically acceptable salt thereof.

5. An amidinoaniline compound according to claim 4, wherein:
ring A is a benzene ring,
R³ is formula (4-1),
Z¹ is a single bond, and
R⁴ is a C₂₋₈ nitrogen-containing nonaromatic heterocyclic group bonded by a nitrogen,
or a pharmaceutically acceptable salt thereof.

6. An amidinoaniline compound according to claim 1, which is represented by formula (1-2): 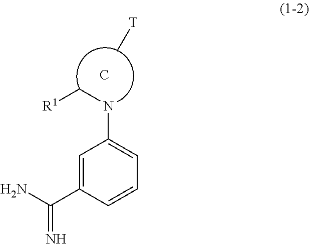
wherein:
ring C is a C₂₋₁₀ nitrogen-containing heteroaryl ring, or a C₂₋₈ nitrogen-containing nonaromatic heterocycle,
T is a hydrogen atom, a hydroxyl group, an amino group, a C₁₋₁₀ alkyl group optionally having substituent(s), a C₁₋₁₀ is alkoxy group optionally having substituent(s), a C₁₋₁₀ alkylamino group optionally having substituent(s), or a C₁₋₁₀ carbamoyloxy group optionally having substituent(s),
or a pharmaceutically acceptable salt thereof.

7. An amidinoaniline compound according to claim 6, wherein:
ring A is a benzene ring, a pyridine ring, a thiophene ring, a piperidine ring, or a piperazine ring, and
V¹ is a hydrogen atom, a halogen atom, a C₁₋₆ alkyl group, or a C₁₋₆ alkoxy group,
or a pharmaceutically acceptable salt thereof.

8. An amidinoaniline derivative according to claim 7, wherein:
(1) R⁴ is an amino group, a C₁₋₁₀ alkylamino group, or a C₂₋₈ nitrogen-containing nonaromatic heterocyclic group bonded by a nitrogen; or
(2) ring B is a C₂₋₈ nitrogen-containing nonaromatic heterocycle,
Z² is an oxygen atom, a sulfur atom or a methylene group, and
V² is a hydrogen atom, a halogen atom, an amidino group, or a C₁₋₆ alkyl group optionally having an imino group at the 1-position,
or a pharmaceutically acceptable salt thereof.

9. An amidinoaniline compound according to claim 8, wherein:
ring A is a benzene ring,
R³ is formula (4-1),
Z¹ is a single bond, and
R⁴ is a C₂₋₈ nitrogen-containing nonaromatic heterocyclic group bonded by a nitrogen,
or a pharmaceutically acceptable salt thereof.

10. An amidinoaniline compound according to claim 1, which is represented by formula (1-3): 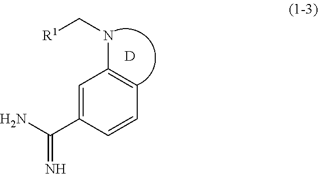
wherein:
ring D is a C₂₋₁₀ nitrogen-containing heteroaryl ring, or a C₂₋₈ nitrogen-containing nonaromatic heterocycle,
or a pharmaceutically acceptable salt thereof.

11. An amidinoaniline compound according to claim 10, wherein:
ring A is a benzene ring, a pyridine ring, a thiophene ring, a piperidine ring, or a piperazine ring, and
V¹ is a hydrogen atom, a halogen atom, a C₁₋₆ alkyl group, or a C₁₋₆ alkoxy group,
or a pharmaceutically acceptable salt thereof.

12. An amidinoaniline derivative according to claim 11, wherein:
(1) R⁴ is an amino group, a C₁₋₁₀ alkylamino group, or a C₂₋₈ nitrogen-containing nonaromatic heterocyclic group bonded by a nitrogen; or
(2) ring B is a C₂₋₈ nitrogen-containing nonaromatic heterocycle,
Z² is an oxygen atom, a sulfur atom or a methylene group, and
V² is a hydrogen atom, a halogen atom, an amidino group, or a C₁₋₆ alkyl group optionally having an imino group at the 1-position,
or a pharmaceutically acceptable salt thereof.

13. An amidinoaniline compound according to claim 12, wherein:
ring A is a benzene ring,
R³ is formula (4-1),
Z¹ is a single bond, and
R⁴ is a C₂₋₈ nitrogen-containing nonaromatic heterocyclic group bonded by a nitrogen,
or a pharmaceutically acceptable salt thereof.

14. An amidinoaniline compound represented by formula (1-1): 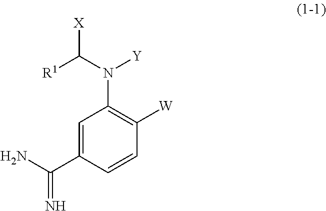
wherein:
X is a hydrogen atom, or a C₁₋₁₀ alkyl group optionally having substituent(s),
Y is a hydrogen atom, a C₁₋₁₀ alkyl group optionally having substituent(s), or an acyl group optionally having substituent(s),
W is a hydrogen atom, a hydroxyl group, an amino group, a C₁₋₁₀ alkyl group optionally having substituent(s), a C₁₋₁₀ alkoxy group optionally having substituent(s), a C₁₋₁₀ acyloxy group optionally having substituent(s), a carbamoyloxy group optionally having substituent(s), a C₁₋₁₀ alkylamino group optionally having substituent(s), a C₁₋₁₀ alkylthio group optionally having substituent(s), a C₁₋₁₀ acylamino group optionally having substituent(s), a carboxyl group, a carbamoyl group optionally having substituent(s), a thiocarbamoyl group optionally having substituent(s), a halogen atom, a cyano group, or a nitro group, or
X and Y are optionally bonded to each other to form a nitrogen-containing heterocycle optionally having substituent(s), or
Y and W are optionally bonded to each other to form a nitrogen-containing heterocycle optionally having substituent(s),
R¹ is a group represented by formula (2-1) or (2-2), provided that when R¹ is a group represented by formula (2-2), X is not a hydrogen atom, 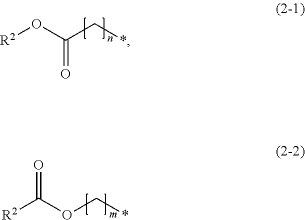
wherein:
n and m are each an integer of 0-2,
R² is a group represented by formula (3′), 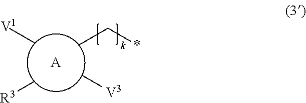
wherein:
k is an integer of 0-2,
ring A is a C₆₋₁₀ aryl ring, a C₁₋₁₀ heteroaryl ring, a C₂₋₈ nitrogen-containing nonaromatic heterocycle, or a C₃₋₁₀ cycloalkyl ring,
V¹ and V³ are the same or different and each is a hydrogen atom, a hydroxyl group, a halogen atom, an amino group, a C₁₋₁₀ alkyl group optionally having substituent(s), a C₁₋₁₀ alkoxy group optionally having substituent(s), a C₁₋₁₀ alkylamino group optionally having substituent(s), a C₁₋₁₀ alkylthio group optionally having substituent(s), a cyano group, a nitro group, a carboxyl group, a carbamoyl group optionally having substituent(s) or a C₂₋₁₀ alkoxycarbonyl group optionally having substituent(s),
R³ is a group represented by formula (4-1) or (4-2): 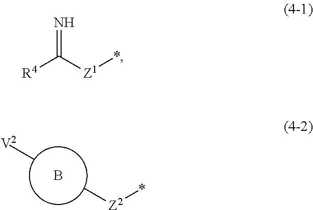
wherein:
Z¹ is —NH—, or a single bond,
R⁴ is a C₁₋₆ alkyl group, an amino group optionally substituted by a C₁₋₁₀ alkyl group, or a C₂₋₈ nitrogen-containing nonaromatic heterocyclic group bonded by a nitrogen, in formula (4-2),
ring B is a C₁₋₁₀ heteroaryl ring, or a C₂₋₈ nitrogen-containing nonaromatic heterocycle,
Z² is a single bond, —NH— optionally substituted by a C₁₋₆ alkyl group, an oxygen atom, a sulfur atom, a methylene group, or —CO—, and
V² is a hydrogen atom, a halogen atom, an amidino group optionally substituted by a C₁₋₆ alkyl group, a guanidino group optionally substituted by a C₁₋₆ alkyl group, or a C₁₋₆ alkyl group optionally having an imino group at the 1-position,
or a pharmaceutically acceptable salt thereof.

15. An activated blood coagulation factor X inhibitor, comprising an amidinoaniline compound according to claim 1 or a pharmaceutically acceptable salt thereof.

16. A pharmaceutical composition, comprising an amidinoaniline compound according to claim 1 or a pharmaceutically acceptable salt thereof and a pharmaceutically acceptable carrier.

17. A pharmaceutical composition according to claim 16, which is an anti-blood coagulant.

18. A pharmaceutical composition according to claim 17, which is an anti-blood coagulant for an extracorporeal blood circulation circuit.

19. A pharmaceutical composition according to claim 17, which is an anti-blood coagulant for hemodialysis.

20. A dialysis solution or dialysis concentrate, comprising an amidinoaniline compound according to claim 1 or a pharmaceutically acceptable salt thereof.

21. An anti-blood coagulant for an extracorporeal blood circulation circuit, which comprises a low molecular weight FXa inhibitor as an active ingredient.

22. An anti-blood coagulant according to claim 21, wherein said low molecular weight FXa inhibitor rapidly disappears from the blood.

23. An anti-blood coagulant according to claim 22, wherein said low molecular weight FXa inhibitor is an FXa selective inhibitor.

24. A method for inhibiting activated blood coagulation factor X, comprising contacting activated blood coagulation factor X with an effective amount of an amidinoaniline compound according to claim 1 or a pharmaceutically acceptable salt thereof.

25. A method for inhibiting coagulation of blood in an extracorporeal blood circulation circuit, which comprises a contacting blood in an extracorporeal blood circulation circuit with an effective amount of a low molecular weight FXa inhibitor.
